SEQUENCE LISTING (48 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - DNA, 6996 bp, Homo sapiens
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 3 - protein, 1438 aa, Artificial sequence
  FVIII SQ amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - DNA, 4314 bp, Artificial sequence
  Codop19 nucleotide sequence encoding FVIII SQ nucleotide sequence
gctactagaagatattatcttggggcagtggagctgagctgggactacatgcagtctgacctgggagaactgcctgtggatgccagatttccccctcgagtgcccaagagcttcccctttaacacctcagtggtgtacaagaagaccctgtttgtggagtttacagaccatctcttcaacattgctaagcccagacctccctggatgggcctgctgggccctaccatccaagctgaagtgtatgacactgttgtgatcacactcaagaacatggcctcccatcctgtgtccctgcatgcagtgggagtctcctactggaaggcctcagaaggagcagagtatgatgaccagaccagccagagagagaaggaggatgacaaggtgtttcctggagggagccacacctatgtgtggcaggtgctgaaggagaatggacctatggccagtgaccctctgtgtcttacctattcctacctgtcacatgtggatctggtgaaggacctgaacagtggcctgattggggctctgctggtttgcagagaaggcagcttggccaaggagaagacccaaaccctgcacaagttcatcctgctgtttgctgtgtttgatgaggggaaatcatggcactcagagaccaagaacagcctcatgcaggatagggatgctgccagtgccagggcttggcccaagatgcacactgtgaatggctatgtgaatagaagcctgcctgggctgataggctgtcacagaaaatctgtgtactggcatgtgattggcatgggcaccacacctgaggtgcactccattttcctggagggccacaccttccttgtgagaaaccacagacaagcttccctggagatcagcccaatcacctttctgactgctcaaaccctcctgatggatctgggccagttcctgctgttctgtcatatctcctcacaccagcatgatggaatggaagcttatgtcaaggtggactcctgcccagaggaaccacagctcagaatgaagaacaatgaggaggctgaggactatgatgatgacctgacagactctgaaatggatgtggtcagatttgatgatgacaacagcccttcattcatccaaatcagatctgtggccaagaagcatcccaagacctgggtgcactacatagctgctgaggaggaggactgggactatgcccctctggtcctggcccctgatgacagaagctataaaagccagtacctgaataatggcccccagagaattggcagaaagtacaagaaagtcagattcatggcttacactgatgagaccttcaaaaccagggaagccatccagcatgagtcaggcatcctgggccccctgctgtatggggaggttggagataccctgctgattatcttcaaaaaccaggcaagcaggccctacaatatctaccctcatggcatcactgatgtcaggccactgtattccagaagactgcctaagggggtgaagcacctgaaggacttcccaatcctgccaggggagattttcaaatacaagtggacagtgactgtggaggatggaccaaccaagtcagatcctagatgtctgaccagatactactccagctttgtgaacatggagagagacctggcctctggcctgattggccctctgctgatctgctataaagagtcagtggaccagagaggcaaccagatcatgagtgacaaaagaaatgtgatcttgttctcagtgtttgatgagaatagatcttggtacctcacagaaaacatccagaggttcctgcccaatccagctggggtgcagctggaagatccagaattccaggccagcaacatcatgcatagcatcaatggttatgtctttgacagcctgcagctgtcagtgtgtctgcatgaagttgcttactggtatattctgtccattggagcccagacagacttcctgtctgtcttcttctctggctacacctttaaacacaagatggtgtatgaggacaccctgaccctgttccctttctctggggaaacagtgttcatgtccatggaaaaccctggactgtggatcctgggctgccataacagtgacttcagaaacagaggcatgacagccctgctcaaggtgtccagctgtgataagaacacaggagactactatgaggatagctatgaggacatcagtgcttacctgctgagcaagaataatgccattgaacccaggtcattttcccaaaatccccctgtgctgaaaaggcaccagagggagatcacgcgtaccaccctgcagagtgaccaggaggaaattgattatgatgacaccatctctgtggaaatgaaaaaggaggattttgacatctatgatgaggatgagaaccagagccctagaagcttccagaaaaagactagacactacttcattgctgcagtggagagactctgggattatggcatgagctccagcccccatgtgctgagaaatagagctcagagtggcagtgtgccacagttcaagaaggtggtgtttcaggagttcactgatggctccttcacacaaccactttacagaggagaactgaatgagcacctgggcctcctgggcccctacatcagggctgaagtggaggataacattatggtcacatttaggaatcaggcttccagaccctactccttttattcctcactcatttcctatgaggaggaccagaggcagggagctgagcccagaaaaaattttgtgaaacccaatgaaaccaagacctacttctggaaggtgcagcaccatatggcccctaccaaggatgaatttgactgcaaggcttgggcttacttttctgatgtggaccttgagaaagatgtgcattcaggcctcattgggccactgctggtgtgccacaccaatacactgaaccctgctcatgggagacaggtcacagtgcaggagtttgcactcttctttaccatctttgatgagaccaagtcctggtatttcactgagaacatggagaggaactgcagggccccttgtaacatccagatggaggatcccaccttcaaggaaaactacagattccatgccatcaatggctacatcatggacaccctgccaggcctggtgatggcccaggaccagaggatcaggtggtacctcctgtctatgggcagcaatgaaaatatccacagcattcacttctctggacatgtgtttactgtgaggaagaaggaggaatacaagatggctctgtacaacctctaccctggggtgtttgaaacagtggagatgctgccctccaaggctggcatctggagagtggaatgtctgattggggagcatctgcatgctggcatgagcacactgttcctggtgtattccaacaagtgccagaccccactgggcatggcctcaggacatatcagggacttccagatcactgctagtggacaatatggacagtgggcacccaagctggccagactgcactactcaggctccatcaatgcctggagtaccaaggagcccttcagctggatcaaggtggacctgctggcccccatgattatacatggcatcaagacccagggagctagacagaagttcagctccctgtacatctcccaattcatcatcatgtactctctggatggcaagaaatggcagacctacagaggcaatagcactggcaccctgatggtgttttttggaaatgttgactcttctggcatcaagcacaacatcttcaacccccccatcattgccagatatatcaggctccaccccacccactactccataaggagcaccctgagaatggagctgatgggctgtgacctgaattcctgctccatgcccctgggcatggaatccaaggcaatctctgatgcacagatcacagcctcctcctacttcaccaacatgtttgcaacctggagcccctccaaggccagactgcacctgcagggcaggtccaatgcttggagaccacaagtgaacaacccaaaggagtggctgcaggtggacttccagaagaccatgaaagtgactggagtgaccacccagggagtgaaatccctgctcactagcatgtatgtgaaggaattcctgatcagtagctctcaagatggccaccagtggaccctgttcttccagaatggcaaggtgaaggtgtttcagggcaaccaggattccttcacccctgtggtgaatagcctggatcccccactgctgaccagatacctgagaatccacccccagtcctgggttcaccagattgccctgagaatggaggtgctgggctgtgaggcccaggacctgtac

SEQ ID NO: 5 - protein, 1424 aa, Artificial sequence
  FVIII RE amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 6 - DNA, 4182 bp, Artificial sequence
  Codop19 nucleotide sequence encoding FVIII polypeptide 96-106
gctactagaagatattatcttggggcagtggagctgagctgggactacatgcagtctgacctgggagaactgcctgtggatgccagatttccccctcgagtgcccaagagcttcccctttaacacctcagtggtgtacaagaagaccctgtttgtggagtttacagaccatctcttcaacattgctaagcccagacctccctggatgggcctgctgggccctaccatccaagctgaagtgtatgacactgttgtgatcacactcaagaacatggcctcccatcctgtgtccctgcatgcagtgggagtctcctactggaaggcctcagaaggagcagagtatgatgaccagaccagccagagagagaaggaggatgacaaggtgtttcctggagggagccacacctatgtgtggcaggtgctgaaggagaatggacctatggccagtgaccctctgtgtcttacctattcctacctgtcacatgtggatctggtgaaggacctgaacagtggcctgattggggctctgctggtttgcagagaaggcagcttggccaaggagaagacccaaaccctgcacaagttcatcctgctgtttgctgtgtttgatgaggggaaatcatggcactcagagaccaagaacagcctcatgcaggatagggatgctgccagtgccagggcttggcccaagatgcacactgtgaatggctatgtgaatagaagcctgcctgggctgataggctgtcacagaaaatctgtgtactggcatgtgattggcatgggcaccacacctgaggtgcactccattttcctggagggccacaccttccttgtgagaaaccacagacaagcttccctggagatcagcccaatcacctttctgactgctcaaaccctcctgatggatctgggccagttcctgctgttctgtcatatctcctcacaccagcatgatggaatggaagcttatgtcaaggtggactcctgcccagaggaaccacagctcagaatgaagaacaatgaggaggctgaggactatgatgatgacctgacagactctgaaatggatgtggtcagatttgatgatgacaacagcccttcattcatccaaatcagatctgtggccaagaagcatcccaagacctgggtgcactacatagctgctgaggaggaggactgggactatgcccctctggtcctggcccctgatgacagaagctataaaagccagtacctgaataatggcccccagagaattggcagaaagtacaagaaagtcagattcatggcttacactgatgagaccttcaaaaccagggaagccatccagcatgagtcaggcatcctgggccccctgctgtatggggaggttggagataccctgctgattatcttcaaaaaccaggcaagcaggccctacaatatctaccctcatggcatcactgatgtcaggccactgtattccagaagactgcctaagggggtgaagcacctgaaggacttcccaatcctgccaggggagattttcaaatacaagtggacagtgactgtggaggatggaccaaccaagtcagatcctagatgtctgaccagatactactccagctttgtgaacatggagagagacctggcctctggcctgattggccctctgctgatctgctataaagagtcagtggaccagagaggcaaccagatcatgagtgacaaaagaaatgtgatcttgttctcagtgtttgatgagaatagatcttggtacctcacagaaaacatccagaggttcctgcccaatccagctggggtgcagctggaagatccagaattccaggccagcaacatcatgcatagcatcaatggttatgtctttgacagcctgcagctgtcagtgtgtctgcatgaagttgcttactggtatattctgtccattggagcccagacagacttcctgtctgtcttcttctctggctacacctttaaacacaagatggtgtatgaggacaccctgaccctgttccctttctctggggaaacagtgttcatgtccatggaaaaccctggactgtggatcctgggctgccataacagtgacttcagaaacagaggcatgacagccctgctcaaggtgtccagctgtgataagaacacaggagactactatgaggatagctatgaggacatcagtgcttacctgctggtggaaatgaaaaaggaggattttgacatctatgatgaggatgagaaccagagccctagaagcttccagaaaaagactagacactacttcattgctgcagtggagagactctgggattatggcatgagctccagcccccatgtgctgagaaatagagctcagagtggcagtgtgccacagttcaagaaggtggtgtttcaggagttcactgatggctccttcacacaaccactttacagaggagaactgaatgagcacctgggcctcctgggcccctacatcagggctgaagtggaggataacattatggtcacatttaggaatcaggcttccagaccctactccttttattcctcactcatttcctatgaggaggaccagaggcagggagctgagcccagaaaaaattttgtgaaacccaatgaaaccaagacctacttctggaaggtgcagcaccatatggcccctaccaaggatgaatttgactgcaaggcttgggcttacttttctgatgtggaccttgagaaagatgtgcattcaggcctcattgggccactgctggtgtgccacaccaatacactgaaccctgctcatgggagacaggtcacagtgcaggagtttgcactcttctttaccatctttgatgagaccaagtcctggtatttcactgagaacatggagaggaactgcagggccccttgtaacatccagatggaggatcccaccttcaaggaaaactacagattccatgccatcaatggctacatcatggacaccctgccaggcctggtgatggcccaggaccagaggatcaggtggtacctcctgtctatgggcagcaatgaaaatatccacagcattcacttctctggacatgtgtttactgtgaggaagaaggaggaatacaagatggctctgtacaacctctaccctggggtgtttgaaacagtggagatgctgccctccaaggctggcatctggagagtggaatgtctgattggggagcatctgcatgctggcatgagcacactgttcctggtgtattccaacaagtgccagaccccactgggcatggcctcaggacatatcagggacttccagatcactgctagtggacaatatggacagtgggcacccaagctggccagactgcactactcaggctccatcaatgcctggagtaccaaggagcccttcagctggatcaaggtggacctgctggcccccatgattatacatggcatcaagacccagggagctagacagaagttcagctccctgtacatctcccaattcatcatcatgtactctctggatggcaagaaatggcagacctacagaggcaatagcactggcaccctgatggtgttttttggaaatgttgactcttctggcatcaagcacaacatcttcaacccccccatcattgccagatatatcaggctccaccccacccactactccataaggagcaccctgagaatggagctgatgggctgtgacctgaattcctgctccatgcccctgggcatggaatccaaggcaatctctgatgcacagatcacagcctcctcctacttcaccaacatgtttgcaacctggagcccctccaaggccagactgcacctgcagggcaggtccaatgcttggagaccacaagtgaacaacccaaaggagtggctgcaggtggacttccagaagaccatgaaagtgactggagtgaccacccagggagtgaaatccctgctcactagcatgtatgtgaaggaattcctgatcagtagctctcaagatggccaccagtggaccctgttcttccagaatggcaaggtgaaggtgtttcagggcaaccaggattccttcacccctgtggtgaatagcctggatcccccactgctgaccagatacctgagaatccacccccagtcctgggttcaccagattgccctgagaatggaggtgctgggctgtgaggcccaggacctgtac

SEQ ID NO: 7 - protein, 1394 aa, Artificial sequence
  FVIII polypeptide 96-106 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 8 - protein, 1386 aa, Artificial sequence
  FVIII polypeptide 99-106 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLVEMKKEDFDIYRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 9 - protein, 1385 aa, Artificial sequence
  FVIII polypeptide 96-109 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 10 - protein, 1377 aa, Artificial sequence
  FVIII polypeptide 99-109 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSVEMKKEDFDIYRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 11 - protein, 1391 aa, Artificial sequence
  FVIII polypeptide 96-107 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 12 - protein, 1394 aa, Artificial sequence
  FVIII polypeptide 107-117 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISATISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 13 - DNA, 119 bp, Artificial sequence
  FRE72 transcription regulatory element
cccagccagtggacttagcccctgtttgctcctccgataactggggtgaccttggttaatattcaccagcagcctccccctcagcttcaggcaccaccactgacctgggacagtgaatc

SEQ ID NO: 14 - DNA, 335 bp, Artificial sequence
  HLP2 transcription regulatory element
ccctaaaatgggcaaacattgcaagcagcaaacagcaaacacacagccctccctgcctgctgaccttggagctggggcagaggtcagacacctctctgggcccatgccacctccaactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 15 - protein, 19 aa, Artificial sequence
  Amino acid sequence of native FVIII signal peptide
MQIELSTCFFLCLLRFCFS

SEQ ID NO: 16 - DNA, 57 bp, Artificial sequence
  Wild-type nucleotide sequence encoding native FVIII signal peptide
atgcaaatagagctctccacctgcttctttctgtgccttttgcgattctgctttagt

SEQ ID NO: 17 - DNA, 57 bp, Artificial sequence
  Codon-optimised nucleotide sequence encoding native FVIII signal peptide
atgcagattgagctctccacctgcttcttcctctgcctcttgagattctgtttctct

SEQ ID NO: 18 - protein, 18 aa, Artificial sequence
  Amino acid sequence of SP5
MKLLAATVLLLTICSLEG

SEQ ID NO: 19 - DNA, 54 bp, Artificial sequence
  Nucleotide sequence encoding SP5
atgaagctgctcgcagcaactgtgctactcctcaccatctgcagccttgaagga

SEQ ID NO: 20 - protein, 18 aa, Artificial sequence
  Amino acid sequence of SP10
MKWVWALLLLAALGSGRA

SEQ ID NO: 21 - DNA, 54 bp, Artificial sequence
  Nucleotide sequence encoding SP10
atgaagtgggtgtgggcgctcttgctgttggcggcgctgggcagcggccgcgcg

SEQ ID NO: 22 - DNA, 49 bp, Artificial sequence
  PolyA sequence (SpA; 49 bp)
aataaaagatctttattttcattagatctgtgtgttggttttttgtgtg

SEQ ID NO: 23 - DNA, 145 bp, Artificial sequence
  AAV2 5′ ITR sequence
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 24 - DNA, 145 bp, Artificial sequence
  AAV2 3′ ITR sequence
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 25 - protein, 736 aa, Artificial sequence
  Amino acid sequence of AAV capsid
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 26 - protein, 724 aa, adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 27 - DNA, 4713 bp, Artificial sequence
  FVIII AAV construct 26-96-106
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctttaattaacccagccagtggacttagcccctgtttgctcctccgataactggggtgaccttggttaatattcaccagcagcctccccctcagcttcaggcaccaccactgacctgggacagtgaatcgcgccaccatgaagctgctcgcagcaactgtgctactcctcaccatctgcagccttgaaggagctactagaagatattatcttggggcagtggagctgagctgggactacatgcagtctgacctgggagaactgcctgtggatgccagatttccccctcgagtgcccaagagcttcccctttaacacctcagtggtgtacaagaagaccctgtttgtggagtttacagaccatctcttcaacattgctaagcccagacctccctggatgggcctgctgggccctaccatccaagctgaagtgtatgacactgttgtgatcacactcaagaacatggcctcccatcctgtgtccctgcatgcagtgggagtctcctactggaaggcctcagaaggagcagagtatgatgaccagaccagccagagagagaaggaggatgacaaggtgtttcctggagggagccacacctatgtgtggcaggtgctgaaggagaatggacctatggccagtgaccctctgtgtcttacctattcctacctgtcacatgtggatctggtgaaggacctgaacagtggcctgattggggctctgctggtttgcagagaaggcagcttggccaaggagaagacccaaaccctgcacaagttcatcctgctgtttgctgtgtttgatgaggggaaatcatggcactcagagaccaagaacagcctcatgcaggatagggatgctgccagtgccagggcttggcccaagatgcacactgtgaatggctatgtgaatagaagcctgcctgggctgataggctgtcacagaaaatctgtgtactggcatgtgattggcatgggcaccacacctgaggtgcactccattttcctggagggccacaccttccttgtgagaaaccacagacaagcttccctggagatcagcccaatcacctttctgactgctcaaaccctcctgatggatctgggccagttcctgctgttctgtcatatctcctcacaccagcatgatggaatggaagcttatgtcaaggtggactcctgcccagaggaaccacagctcagaatgaagaacaatgaggaggctgaggactatgatgatgacctgacagactctgaaatggatgtggtcagatttgatgatgacaacagcccttcattcatccaaatcagatctgtggccaagaagcatcccaagacctgggtgcactacatagctgctgaggaggaggactgggactatgcccctctggtcctggcccctgatgacagaagctataaaagccagtacctgaataatggcccccagagaattggcagaaagtacaagaaagtcagattcatggcttacactgatgagaccttcaaaaccagggaagccatccagcatgagtcaggcatcctgggccccctgctgtatggggaggttggagataccctgctgattatcttcaaaaaccaggcaagcaggccctacaatatctaccctcatggcatcactgatgtcaggccactgtattccagaagactgcctaagggggtgaagcacctgaaggacttcccaatcctgccaggggagattttcaaatacaagtggacagtgactgtggaggatggaccaaccaagtcagatcctagatgtctgaccagatactactccagctttgtgaacatggagagagacctggcctctggcctgattggccctctgctgatctgctataaagagtcagtggaccagagaggcaaccagatcatgagtgacaaaagaaatgtgatcttgttctcagtgtttgatgagaatagatcttggtacctcacagaaaacatccagaggttcctgcccaatccagctggggtgcagctggaagatccagaattccaggccagcaacatcatgcatagcatcaatggttatgtctttgacagcctgcagctgtcagtgtgtctgcatgaagttgcttactggtatattctgtccattggagcccagacagacttcctgtctgtcttcttctctggctacacctttaaacacaagtgggtgtatgaggacaccctgaccctgttccctttctctggggaaacagtgttcatgtccatggaaaaccctggactgtggatcctgggctgccataacagtgacttcagaaacagaggcatgacagccctgctcaaggtgtccagctgtgataagaacacaggagactactatgaggatagctatgaggacatcagtgcttacctgctggtggaaatgaaaaaggaggattttgacatctatgatgaggatgagaaccagagccctagaagcttccagaaaaagactagacactacttcattgctgcagtggagagactctgggattatggcatgagctccagcccccatgtgctgagaaatagagctcagagtggcagtgtgccacagttcaagaaggtggtgtttcaggagttcactgatggctccttcacacaaccactttacagaggagaactgaatgagcacctgggcctcctgggcccctacatcagggctgaagtggaggataacattatggtcacatttaggaatcaggcttccagaccctactccttttattcctcactcatttcctatgaggaggaccagaggcagggagctgagcccagaaaaaattttgtgaaacccaatgaaaccaagacctacttctggaaggtgcagcaccatatggcccctaccaaggatgaatttgactgcaaggcttgggcttacttttctgatgtggaccttgagaaagatgtgcattcaggcctcattgggccactgctggtgtgccacaccaatacactgaaccctgctcatgggagacaggtcacagtgcaggagtttgcactcttctttaccatctttgatgagaccaagtcctggtatttcactgagaacatggagaggaactgcagggccccttgtaacatccagatggaggatcccaccttcaaggaaaactacagattccatgccatcaatggctacatcatggacaccctgccaggcctggtgatggcccaggaccagaggatcaggtggtacctcctgtctatgggcagcaatgaaaatatccacagcattcacttctctggacatgtgtttactgtgaggaagaaggaggaatacaagatggctctgtacaacctctaccctggggtgtttgaaacagtggagatgctgccctccaaggctggcatctggagagtggaatgtctgattggggagcatctgcatgctggcatgagcacactgttcctggtgtattccaacaagtgccagaccccactgggcatggcctcaggacatatcagggacttccagatcactgctagtggacaatatggacagtgggcacccaagctggccagactgcactactcaggctccatcaatgcctggagtaccaaggagcccttcagctggatcaaggtggacctgctggcccccatgattatacatggcatcaagacccagggagctagacagaagttcagctccctgtacatctcccaattcatcatcatgtactctctggatggcaagaaatggcagacctacagaggcaatagcactggcaccctgatggtgttttttggaaatgttgactcttctggcatcaagcacaacatcttcaacccccccatcattgccagatatatcaggctccaccccacccactactccataaggagcaccctgagaatggagctgatgggctgtgacctgaattcctgctccatgcccctgggcatggaatccaaggcaatctctgatgcacagatcacagcctcctcctacttcaccaacatgtttgcaacctggagcccctccaaggccagactgcacctgcagggcaggtccaatgcttggagaccacaagtgaacaacccaaaggagtggctgcaggtggacttccagaagaccatgaaagtgactggagtgaccacccagggagtgaaatccctgctcactagcatgtatgtgaaggaattcctgatcagtagctctcaagatggccaccagtggaccctgttcttccagaatggcaaggtgaaggtgtttcagggcaaccaggattccttcacccctgtggtgaatagcctggatcccccactgctgaccagatacctgagaatccacccccagtcctgggttcaccagattgccctgagaatggaggtgctgggctgtgaggcccaggacctgtactgaaataaaagatctttattttcattagatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 28 - protein, 1394 aa, Artificial sequence
  FVIII 26-96-106 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKWVYEDTLTLFPFSGETVFMSMENPGLWILGCHNSDFRNRGMTALLKVSSCDKNTGDYYEDSYEDISAYLLVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 29 - DNA, 4182 bp, Artificial sequence
  Codop19 nucleotide sequence encoding 26-96-106
gctactagaagatattatcttggggcagtggagctgagctgggactacatgcagtctgacctgggagaactgcctgtggatgccagatttccccctcgagtgcccaagagcttcccctttaacacctcagtggtgtacaagaagaccctgtttgtggagtttacagaccatctcttcaacattgctaagcccagacctccctggatgggcctgctgggccctaccatccaagctgaagtgtatgacactgttgtgatcacactcaagaacatggcctcccatcctgtgtccctgcatgcagtgggagtctcctactggaaggcctcagaaggagcagagtatgatgaccagaccagccagagagagaaggaggatgacaaggtgtttcctggagggagccacacctatgtgtggcaggtgctgaaggagaatggacctatggccagtgaccctctgtgtcttacctattcctacctgtcacatgtggatctggtgaaggacctgaacagtggcctgattggggctctgctggtttgcagagaaggcagcttggccaaggagaagacccaaaccctgcacaagttcatcctgctgtttgctgtgtttgatgaggggaaatcatggcactcagagaccaagaacagcctcatgcaggatagggatgctgccagtgccagggcttggcccaagatgcacactgtgaatggctatgtgaatagaagcctgcctgggctgataggctgtcacagaaaatctgtgtactggcatgtgattggcatgggcaccacacctgaggtgcactccattttcctggagggccacaccttccttgtgagaaaccacagacaagcttccctggagatcagcccaatcacctttctgactgctcaaaccctcctgatggatctgggccagttcctgctgttctgtcatatctcctcacaccagcatgatggaatggaagcttatgtcaaggtggactcctgcccagaggaaccacagctcagaatgaagaacaatgaggaggctgaggactatgatgatgacctgacagactctgaaatggatgtggtcagatttgatgatgacaacagcccttcattcatccaaatcagatctgtggccaagaagcatcccaagacctgggtgcactacatagctgctgaggaggaggactgggactatgcccctctggtcctggcccctgatgacagaagctataaaagccagtacctgaataatggcccccagagaattggcagaaagtacaagaaagtcagattcatggcttacactgatgagaccttcaaaaccagggaagccatccagcatgagtcaggcatcctgggccccctgctgtatggggaggttggagataccctgctgattatcttcaaaaaccaggcaagcaggccctacaatatctaccctcatggcatcactgatgtcaggccactgtattccagaagactgcctaagggggtgaagcacctgaaggacttcccaatcctgccaggggagattttcaaatacaagtggacagtgactgtggaggatggaccaaccaagtcagatcctagatgtctgaccagatactactccagctttgtgaacatggagagagacctggcctctggcctgattggccctctgctgatctgctataaagagtcagtggaccagagaggcaaccagatcatgagtgacaaaagaaatgtgatcttgttctcagtgtttgatgagaatagatcttggtacctcacagaaaacatccagaggttcctgcccaatccagctggggtgcagctggaagatccagaattccaggccagcaacatcatgcatagcatcaatggttatgtctttgacagcctgcagctgtcagtgtgtctgcatgaagttgcttactggtatattctgtccattggagcccagacagacttcctgtctgtcttcttctctggctacacctttaaacacaagtgggtgtatgaggacaccctgaccctgttccctttctctggggaaacagtgttcatgtccatggaaaaccctggactgtggatcctgggctgccataacagtgacttcagaaacagaggcatgacagccctgctcaaggtgtccagctgtgataagaacacaggagactactatgaggatagctatgaggacatcagtgcttacctgctggtggaaatgaaaaaggaggattttgacatctatgatgaggatgagaaccagagccctagaagcttccagaaaaagactagacactacttcattgctgcagtggagagactctgggattatggcatgagctccagcccccatgtgctgagaaatagagctcagagtggcagtgtgccacagttcaagaaggtggtgtttcaggagttcactgatggctccttcacacaaccactttacagaggagaactgaatgagcacctgggcctcctgggcccctacatcagggctgaagtggaggataacattatggtcacatttaggaatcaggcttccagaccctactccttttattcctcactcatttcctatgaggaggaccagaggcagggagctgagcccagaaaaaattttgtgaaacccaatgaaaccaagacctacttctggaaggtgcagcaccatatggcccctaccaaggatgaatttgactgcaaggcttgggcttacttttctgatgtggaccttgagaaagatgtgcattcaggcctcattgggccactgctggtgtgccacaccaatacactgaaccctgctcatgggagacaggtcacagtgcaggagtttgcactcttctttaccatctttgatgagaccaagtcctggtatttcactgagaacatggagaggaactgcagggccccttgtaacatccagatggaggatcccaccttcaaggaaaactacagattccatgccatcaatggctacatcatggacaccctgccaggcctggtgatggcccaggaccagaggatcaggtggtacctcctgtctatgggcagcaatgaaaatatccacagcattcacttctctggacatgtgtttactgtgaggaagaaggaggaatacaagatggctctgtacaacctctaccctggggtgtttgaaacagtggagatgctgccctccaaggctggcatctggagagtggaatgtctgattggggagcatctgcatgctggcatgagcacactgttcctggtgtattccaacaagtgccagaccccactgggcatggcctcaggacatatcagggacttccagatcactgctagtggacaatatggacagtgggcacccaagctggccagactgcactactcaggctccatcaatgcctggagtaccaaggagcccttcagctggatcaaggtggacctgctggcccccatgattatacatggcatcaagacccagggagctagacagaagttcagctccctgtacatctcccaattcatcatcatgtactctctggatggcaagaaatggcagacctacagaggcaatagcactggcaccctgatggtgttttttggaaatgttgactcttctggcatcaagcacaacatcttcaacccccccatcattgccagatatatcaggctccaccccacccactactccataaggagcaccctgagaatggagctgatgggctgtgacctgaattcctgctccatgcccctgggcatggaatccaaggcaatctctgatgcacagatcacagcctcctcctacttcaccaacatgtttgcaacctggagcccctccaaggccagactgcacctgcagggcaggtccaatgcttggagaccacaagtgaacaacccaaaggagtggctgcaggtggacttccagaagaccatgaaagtgactggagtgaccacccagggagtgaaatccctgctcactagcatgtatgtgaaggaattcctgatcagtagctctcaagatggccaccagtggaccctgttcttccagaatggcaaggtgaaggtgtttcagggcaaccaggattccttcacccctgtggtgaatagcctggatcccccactgctgaccagatacctgagaatccacccccagtcctgggttcaccagattgccctgagaatggaggtgctgggctgtgaggcccaggacctgtac

SEQ ID NO: 30 - protein, 1438 aa, Artificial sequence
  FVIII 26-SQ amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKWVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 31 - DNA, 4314 bp, Artificial sequence
  Codop19 nucleotide sequence encoding FVIII polypeptide 26 SQ
gctactagaagatattatcttggggcagtggagctgagctgggactacatgcagtctgacctgggagaactgcctgtggatgccagatttccccctcgagtgcccaagagcttcccctttaacacctcagtggtgtacaagaagaccctgtttgtggagtttacagaccatctcttcaacattgctaagcccagacctccctggatgggcctgctgggccctaccatccaagctgaagtgtatgacactgttgtgatcacactcaagaacatggcctcccatcctgtgtccctgcatgcagtgggagtctcctactggaaggcctcagaaggagcagagtatgatgaccagaccagccagagagagaaggaggatgacaaggtgtttcctggagggagccacacctatgtgtggcaggtgctgaaggagaatggacctatggccagtgaccctctgtgtcttacctattcctacctgtcacatgtggatctggtgaaggacctgaacagtggcctgattggggctctgctggtttgcagagaaggcagcttggccaaggagaagacccaaaccctgcacaagttcatcctgctgtttgctgtgtttgatgaggggaaatcatggcactcagagaccaagaacagcctcatgcaggatagggatgctgccagtgccagggcttggcccaagatgcacactgtgaatggctatgtgaatagaagcctgcctgggctgataggctgtcacagaaaatctgtgtactggcatgtgattggcatgggcaccacacctgaggtgcactccattttcctggagggccacaccttccttgtgagaaaccacagacaagcttccctggagatcagcccaatcacctttctgactgctcaaaccctcctgatggatctgggccagttcctgctgttctgtcatatctcctcacaccagcatgatggaatggaagcttatgtcaaggtggactcctgcccagaggaaccacagctcagaatgaagaacaatgaggaggctgaggactatgatgatgacctgacagactctgaaatggatgtggtcagatttgatgatgacaacagcccttcattcatccaaatcagatctgtggccaagaagcatcccaagacctgggtgcactacatagctgctgaggaggaggactgggactatgcccctctggtcctggcccctgatgacagaagctataaaagccagtacctgaataatggcccccagagaattggcagaaagtacaagaaagtcagattcatggcttacactgatgagaccttcaaaaccagggaagccatccagcatgagtcaggcatcctgggccccctgctgtatggggaggttggagataccctgctgattatcttcaaaaaccaggcaagcaggccctacaatatctaccctcatggcatcactgatgtcaggccactgtattccagaagactgcctaagggggtgaagcacctgaaggacttcccaatcctgccaggggagattttcaaatacaagtggacagtgactgtggaggatggaccaaccaagtcagatcctagatgtctgaccagatactactccagctttgtgaacatggagagagacctggcctctggcctgattggccctctgctgatctgctataaagagtcagtggaccagagaggcaaccagatcatgagtgacaaaagaaatgtgatcttgttctcagtgtttgatgagaatagatcttggtacctcacagaaaacatccagaggttcctgcccaatccagctggggtgcagctggaagatccagaattccaggccagcaacatcatgcatagcatcaatggttatgtctttgacagcctgcagctgtcagtgtgtctgcatgaagttgcttactggtatattctgtccattggagcccagacagacttcctgtctgtcttcttctctggctacacctttaaacacaagtgggtgtatgaggacaccctgaccctgttccctttctctggggaaacagtgttcatgtccatggaaaaccctggactgtggatcctgggctgccataacagtgacttcagaaacagaggcatgacagccctgctcaaggtgtccagctgtgataagaacacaggagactactatgaggatagctatgaggacatcagtgcttacctgctgagcaagaataatgccattgaacccaggtcattttcccaaaatccccctgtgctgaaaaggcaccagagggagatcacgcgtaccaccctgcagagtgaccaggaggaaattgattatgatgacaccatctctgtggaaatgaaaaaggaggattttgacatctatgatgaggatgagaaccagagccctagaagcttccagaaaaagactagacactacttcattgctgcagtggagagactctgggattatggcatgagctccagcccccatgtgctgagaaatagagctcagagtggcagtgtgccacagttcaagaaggtggtgtttcaggagttcactgatggctccttcacacaaccactttacagaggagaactgaatgagcacctgggcctcctgggcccctacatcagggctgaagtggaggataacattatggtcacatttaggaatcaggcttccagaccctactccttttattcctcactcatttcctatgaggaggaccagaggcagggagctgagcccagaaaaaattttgtgaaacccaatgaaaccaagacctacttctggaaggtgcagcaccatatggcccctaccaaggatgaatttgactgcaaggcttgggcttacttttctgatgtggaccttgagaaagatgtgcattcaggcctcattgggccactgctggtgtgccacaccaatacactgaaccctgctcatgggagacaggtcacagtgcaggagtttgcactcttctttaccatctttgatgagaccaagtcctggtatttcactgagaacatggagaggaactgcagggccccttgtaacatccagatggaggatcccaccttcaaggaaaactacagattccatgccatcaatggctacatcatggacaccctgccaggcctggtgatggcccaggaccagaggatcaggtggtacctcctgtctatgggcagcaatgaaaatatccacagcattcacttctctggacatgtgtttactgtgaggaagaaggaggaatacaagatggctctgtacaacctctaccctggggtgtttgaaacagtggagatgctgccctccaaggctggcatctggagagtggaatgtctgattggggagcatctgcatgctggcatgagcacactgttcctggtgtattccaacaagtgccagaccccactgggcatggcctcaggacatatcagggacttccagatcactgctagtggacaatatggacagtgggcacccaagctggccagactgcactactcaggctccatcaatgcctggagtaccaaggagcccttcagctggatcaaggtggacctgctggcccccatgattatacatggcatcaagacccagggagctagacagaagttcagctccctgtacatctcccaattcatcatcatgtactctctggatggcaagaaatggcagacctacagaggcaatagcactggcaccctgatggtgttttttggaaatgttgactcttctggcatcaagcacaacatcttcaacccccccatcattgccagatatatcaggctccaccccacccactactccataaggagcaccctgagaatggagctgatgggctgtgacctgaattcctgctccatgcccctgggcatggaatccaaggcaatctctgatgcacagatcacagcctcctcctacttcaccaacatgtttgcaacctggagcccctccaaggccagactgcacctgcagggcaggtccaatgcttggagaccacaagtgaacaacccaaaggagtggctgcaggtggacttccagaagaccatgaaagtgactggagtgaccacccagggagtgaaatccctgctcactagcatgtatgtgaaggaattcctgatcagtagctctcaagatggccaccagtggaccctgttcttccagaatggcaaggtgaaggtgtttcagggcaaccaggattccttcacccctgtggtgaatagcctggatcccccactgctgaccagatacctgagaatccacccccagtcctgggttcaccagattgccctgagaatggaggtgctgggctgtgaggcccaggacctgtac

SEQ ID NO: 32 - DNA, 4845 bp, Artificial sequence
  FVIII AAV construct SQ
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctttaattaacccagccagtggacttagcccctgtttgctcctccgataactggggtgaccttggttaatattcaccagcagcctccccctcagcttcaggcaccaccactgacctgggacagtgaatcgcgccaccatgaagctgctcgcagcaactgtgctactcctcaccatctgcagccttgaaggagctactagaagatattatcttggggcagtggagctgagctgggactacatgcagtctgacctgggagaactgcctgtggatgccagatttccccctcgagtgcccaagagcttcccctttaacacctcagtggtgtacaagaagaccctgtttgtggagtttacagaccatctcttcaacattgctaagcccagacctccctggatgggcctgctgggccctaccatccaagctgaagtgtatgacactgttgtgatcacactcaagaacatggcctcccatcctgtgtccctgcatgcagtgggagtctcctactggaaggcctcagaaggagcagagtatgatgaccagaccagccagagagagaaggaggatgacaaggtgtttcctggagggagccacacctatgtgtggcaggtgctgaaggagaatggacctatggccagtgaccctctgtgtcttacctattcctacctgtcacatgtggatctggtgaaggacctgaacagtggcctgattggggctctgctggtttgcagagaaggcagcttggccaaggagaagacccaaaccctgcacaagttcatcctgctgtttgctgtgtttgatgaggggaaatcatggcactcagagaccaagaacagcctcatgcaggatagggatgctgccagtgccagggcttggcccaagatgcacactgtgaatggctatgtgaatagaagcctgcctgggctgataggctgtcacagaaaatctgtgtactggcatgtgattggcatgggcaccacacctgaggtgcactccattttcctggagggccacaccttccttgtgagaaaccacagacaagcttccctggagatcagcccaatcacctttctgactgctcaaaccctcctgatggatctgggccagttcctgctgttctgtcatatctcctcacaccagcatgatggaatggaagcttatgtcaaggtggactcctgcccagaggaaccacagctcagaatgaagaacaatgaggaggctgaggactatgatgatgacctgacagactctgaaatggatgtggtcagatttgatgatgacaacagcccttcattcatccaaatcagatctgtggccaagaagcatcccaagacctgggtgcactacatagctgctgaggaggaggactgggactatgcccctctggtcctggcccctgatgacagaagctataaaagccagtacctgaataatggcccccagagaattggcagaaagtacaagaaagtcagattcatggcttacactgatgagaccttcaaaaccagggaagccatccagcatgagtcaggcatcctgggccccctgctgtatggggaggttggagataccctgctgattatcttcaaaaaccaggcaagcaggccctacaatatctaccctcatggcatcactgatgtcaggccactgtattccagaagactgcctaagggggtgaagcacctgaaggacttcccaatcctgccaggggagattttcaaatacaagtggacagtgactgtggaggatggaccaaccaagtcagatcctagatgtctgaccagatactactccagctttgtgaacatggagagagacctggcctctggcctgattggccctctgctgatctgctataaagagtcagtggaccagagaggcaaccagatcatgagtgacaaaagaaatgtgatcttgttctcagtgtttgatgagaatagatcttggtacctcacagaaaacatccagaggttcctgcccaatccagctggggtgcagctggaagatccagaattccaggccagcaacatcatgcatagcatcaatggttatgtctttgacagcctgcagctgtcagtgtgtctgcatgaagttgcttactggtatattctgtccattggagcccagacagacttcctgtctgtcttcttctctggctacacctttaaacacaagatggtgtatgaggacaccctgaccctgttccctttctctggggaaacagtgttcatgtccatggaaaaccctggactgtggatcctgggctgccataacagtgacttcagaaacagaggcatgacagccctgctcaaggtgtccagctgtgataagaacacaggagactactatgaggatagctatgaggacatcagtgcttacctgctgagcaagaataatgccattgaacccaggtcattttcccaaaatccccctgtgctgaaaaggcaccagagggagatcacgcgtaccaccctgcagagtgaccaggaggaaattgattatgatgacaccatctctgtggaaatgaaaaaggaggattttgacatctatgatgaggatgagaaccagagccctagaagcttccagaaaaagactagacactacttcattgctgcagtggagagactctgggattatggcatgagctccagcccccatgtgctgagaaatagagctcagagtggcagtgtgccacagttcaagaaggtggtgtttcaggagttcactgatggctccttcacacaaccactttacagaggagaactgaatgagcacctgggcctcctgggcccctacatcagggctgaagtggaggataacattatggtcacatttaggaatcaggcttccagaccctactccttttattcctcactcatttcctatgaggaggaccagaggcagggagctgagcccagaaaaaattttgtgaaacccaatgaaaccaagacctacttctggaaggtgcagcaccatatggcccctaccaaggatgaatttgactgcaaggcttgggcttacttttctgatgtggaccttgagaaagatgtgcattcaggcctcattgggccactgctggtgtgccacaccaatacactgaaccctgctcatgggagacaggtcacagtgcaggagtttgcactcttctttaccatctttgatgagaccaagtcctggtatttcactgagaacatggagaggaactgcagggccccttgtaacatccagatggaggatcccaccttcaaggaaaactacagattccatgccatcaatggctacatcatggacaccctgccaggcctggtgatggcccaggaccagaggatcaggtggtacctcctgtctatgggcagcaatgaaaatatccacagcattcacttctctggacatgtgtttactgtgaggaagaaggaggaatacaagatggctctgtacaacctctaccctggggtgtttgaaacagtggagatgctgccctccaaggctggcatctggagagtggaatgtctgattggggagcatctgcatgctggcatgagcacactgttcctggtgtattccaacaagtgccagaccccactgggcatggcctcaggacatatcagggacttccagatcactgctagtggacaatatggacagtgggcacccaagctggccagactgcactactcaggctccatcaatgcctggagtaccaaggagcccttcagctggatcaaggtggacctgctggcccccatgattatacatggcatcaagacccagggagctagacagaagttcagctccctgtacatctcccaattcatcatcatgtactctctggatggcaagaaatggcagacctacagaggcaatagcactggcaccctgatggtgttttttggaaatgttgactcttctggcatcaagcacaacatcttcaacccccccatcattgccagatatatcaggctccaccccacccactactccataaggagcaccctgagaatggagctgatgggctgtgacctgaattcctgctccatgcccctgggcatggaatccaaggcaatctctgatgcacagatcacagcctcctcctacttcaccaacatgtttgcaacctggagcccctccaaggccagactgcacctgcagggcaggtccaatgcttggagaccacaagtgaacaacccaaaggagtggctgcaggtggacttccagaagaccatgaaagtgactggagtgaccacccagggagtgaaatccctgctcactagcatgtatgtgaaggaattcctgatcagtagctctcaagatggccaccagtggaccctgttcttccagaatggcaaggtgaaggtgtttcagggcaaccaggattccttcacccctgtggtgaatagcctggatcccccactgctgaccagatacctgagaatccacccccagtcctgggttcaccagattgccctgagaatggaggtgctgggctgtgaggcccaggacctgtactgaaataaaagatctttattttcattagatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 33 - DNA, 4845 bp, Artificial sequence
  FVIII AAV construct 26-SQ
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctttaattaacccagccagtggacttagcccctgtttgctcctccgataactggggtgaccttggttaatattcaccagcagcctccccctcagcttcaggcaccaccactgacctgggacagtgaatcgcgccaccatgaagctgctcgcagcaactgtgctactcctcaccatctgcagccttgaaggagctactagaagatattatcttggggcagtggagctgagctgggactacatgcagtctgacctgggagaactgcctgtggatgccagatttccccctcgagtgcccaagagcttcccctttaacacctcagtggtgtacaagaagaccctgtttgtggagtttacagaccatctcttcaacattgctaagcccagacctccctggatgggcctgctgggccctaccatccaagctgaagtgtatgacactgttgtgatcacactcaagaacatggcctcccatcctgtgtccctgcatgcagtgggagtctcctactggaaggcctcagaaggagcagagtatgatgaccagaccagccagagagagaaggaggatgacaaggtgtttcctggagggagccacacctatgtgtggcaggtgctgaaggagaatggacctatggccagtgaccctctgtgtcttacctattcctacctgtcacatgtggatctggtgaaggacctgaacagtggcctgattggggctctgctggtttgcagagaaggcagcttggccaaggagaagacccaaaccctgcacaagttcatcctgctgtttgctgtgtttgatgaggggaaatcatggcactcagagaccaagaacagcctcatgcaggatagggatgctgccagtgccagggcttggcccaagatgcacactgtgaatggctatgtgaatagaagcctgcctgggctgataggctgtcacagaaaatctgtgtactggcatgtgattggcatgggcaccacacctgaggtgcactccattttcctggagggccacaccttccttgtgagaaaccacagacaagcttccctggagatcagcccaatcacctttctgactgctcaaaccctcctgatggatctgggccagttcctgctgttctgtcatatctcctcacaccagcatgatggaatggaagcttatgtcaaggtggactcctgcccagaggaaccacagctcagaatgaagaacaatgaggaggctgaggactatgatgatgacctgacagactctgaaatggatgtggtcagatttgatgatgacaacagcccttcattcatccaaatcagatctgtggccaagaagcatcccaagacctgggtgcactacatagctgctgaggaggaggactgggactatgcccctctggtcctggcccctgatgacagaagctataaaagccagtacctgaataatggcccccagagaattggcagaaagtacaagaaagtcagattcatggcttacactgatgagaccttcaaaaccagggaagccatccagcatgagtcaggcatcctgggccccctgctgtatggggaggttggagataccctgctgattatcttcaaaaaccaggcaagcaggccctacaatatctaccctcatggcatcactgatgtcaggccactgtattccagaagactgcctaagggggtgaagcacctgaaggacttcccaatcctgccaggggagattttcaaatacaagtggacagtgactgtggaggatggaccaaccaagtcagatcctagatgtctgaccagatactactccagctttgtgaacatggagagagacctggcctctggcctgattggccctctgctgatctgctataaagagtcagtggaccagagaggcaaccagatcatgagtgacaaaagaaatgtgatcttgttctcagtgtttgatgagaatagatcttggtacctcacagaaaacatccagaggttcctgcccaatccagctggggtgcagctggaagatccagaattccaggccagcaacatcatgcatagcatcaatggttatgtctttgacagcctgcagctgtcagtgtgtctgcatgaagttgcttactggtatattctgtccattggagcccagacagacttcctgtctgtcttcttctctggctacacctttaaacacaagtgggtgtatgaggacaccctgaccctgttccctttctctggggaaacagtgttctggtccatggaaaaccctggactgtggatcctgggctgccataacagtgacttcagaaacagaggcatgacagccctgctcaaggtgtccagctgtgataagaacacaggagactactatgaggatagctatgaggacatcagtgcttacctgctgagcaagaataatgccattgaacccaggtcattttcccaaaatccccctgtgctgaaaaggcaccagagggagatcacgcgtaccaccctgcagagtgaccaggaggaaattgattatgatgacaccatctctgtggaaatgaaaaaggaggattttgacatctatgatgaggatgagaaccagagccctagaagcttccagaaaaagactagacactacttcattgctgcagtggagagactctgggattatggcatgagctccagcccccatgtgctgagaaatagagctcagagtggcagtgtgccacagttcaagaaggtggtgtttcaggagttcactgatggctccttcacacaaccactttacagaggagaactgaatgagcacctgggcctcctgggcccctacatcagggctgaagtggaggataacattatggtcacatttaggaatcaggcttccagaccctactccttttattcctcactcatttcctatgaggaggaccagaggcagggagctgagcccagaaaaaattttgtgaaacccaatgaaaccaagacctacttctggaaggtgcagcaccatatggcccctaccaaggatgaatttgactgcaaggcttgggcttacttttctgatgtggaccttgagaaagatgtgcattcaggcctcattgggccactgctggtgtgccacaccaatacactgaaccctgctcatgggagacaggtcacagtgcaggagtttgcactcttctttaccatctttgatgagaccaagtcctggtatttcactgagaacatggagaggaactgcagggccccttgtaacatccagatggaggatcccaccttcaaggaaaactacagattccatgccatcaatggctacatcatggacaccctgccaggcctggtgatggcccaggaccagaggatcaggtggtacctcctgtctatgggcagcaatgaaaatatccacagcattcacttctctggacatgtgtttactgtgaggaagaaggaggaatacaagatggctctgtacaacctctaccctggggtgtttgaaacagtggagatgctgccctccaaggctggcatctggagagtggaatgtctgattggggagcatctgcatgctggcatgagcacactgttcctggtgtattccaacaagtgccagaccccactgggcatggcctcaggacatatcagggacttccagatcactgctagtggacaatatggacagtgggcacccaagctggccagactgcactactcaggctccatcaatgcctggagtaccaaggagcccttcagctggatcaaggtggacctgctggcccccatgattatacatggcatcaagacccagggagctagacagaagttcagctccctgtacatctcccaattcatcatcatgtactctctggatggcaagaaatggcagacctacagaggcaatagcactggcaccctgatggtgttttttggaaatgttgactcttctggcatcaagcacaacatcttcaacccccccatcattgccagatatatcaggctccaccccacccactactccataaggagcaccctgagaatggagctgatgggctgtgacctgaattcctgctccatgcccctgggcatggaatccaaggcaatctctgatgcacagatcacagcctcctcctacttcaccaacatgtttgcaacctggagcccctccaaggccagactgcacctgcagggcaggtccaatgcttggagaccacaagtgaacaacccaaaggagtggctgcaggtggacttccagaagaccatgaaagtgactggagtgaccacccagggagtgaaatccctgctcactagcatgtatgtgaaggaattcctgatcagtagctctcaagatggccaccagtggaccctgttcttccagaatggcaaggtgaaggtgtttcagggcaaccaggattccttcacccctgtggtgaatagcctggatcccccactgctgaccagatacctgagaatccacccccagtcctgggttcaccagattgccctgagaatggaggtgctgggctgtgaggcccaggacctgtactgaaataaaagatctttattttcattagatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 34 - DNA, 4970 bp, Artificial sequence
  FVIII AAV construct (SEQ ID NO1 from WO 2017/053677)
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgaaataaaagatctttattttcattagatctgtgtgttggttttttgtgtgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 35 - protein, 1394 aa, Artificial sequence
  FVIII 65-96-106 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCWNSDFRNRGMTALLKVSSCDKNTGDYYEDSYEDISAYLLVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 36 - protein, 1394 aa, Artificial sequence
  FVIII 26-65-96-106 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKWVYEDTLTLFPFSGETVFMSMENPGLWILGCWNSDFRNRGMTALLKVSSCDKNTGDYYEDSYEDISAYLLVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 37 - protein, 1394 aa, Artificial sequence
  FVIII 12SS-96-106 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGCWILGCHNSDFRNRGMTALLKVSSCDKNTGDYYEDSYEDISAYLLVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGCEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 38 - protein, 1394 aa, Artificial sequence
  FVIII 28SS-96-106 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMECPGLWILGCHNSDFRNRGMTALLKVSSCDKNTGDYYEDSYEDISAYLLVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLICYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 39 - protein, 1394 aa, Artificial sequence
  FVIII 31SS-96-106 amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNCDFRNRGMTALLKVSSCDKNTGDYYEDSYEDISAYLLVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLCKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 40 - protein, 1438 aa, Artificial sequence
  FVIII 65-SQ amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCWNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 41 - protein, 1438 aa, Artificial sequence
  FVIII 26-65-SQ amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKWVYEDTLTLFPFSGETVFMSMENPGLWILGCWNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 42 - protein, 1438 aa, Artificial sequence
  FVIII 12SS-SQ amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGCWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGCEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 43 - protein, 1438 aa, Artificial sequence
  FVIII 28SS-SQ amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMECPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLICYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 44 - protein, 1438 aa, Artificial sequence
  FVIII 31SS-SQ amino acid sequence
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNCDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLCKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 45 - protein, 12 aa, Artificial sequence
  Amino acids 656-667 of SEQ ID NO1
YTFKHKMVYEDT

SEQ ID NO: 46 - protein, 12 aa, Artificial sequence
  Amino acids 1823-1834 of SEQ ID NO1
MAPTKDEFDCKA

SEQ ID NO: 47 - protein, 736 aa, Artificial sequence
  Amino acid sequence of LK03 capsid
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 48 - protein, 736 aa, adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

(19) United States
(12) Patent Application Publication
(10) Pub. No.: US 2022/0396610 A1
(43) Pub. Date: Dec. 15, 2022

(54) FACTOR VIII POLYPEPTIDE

(71) Applicant: Freeline Therapeutics Limited, Stevenage (GB)

(72) Inventors: Azadeh KIA, Enfield (GB); Fabrizio COMPER, Stevenage (GB); Romuald CORBAU, Chiswell Green (GB); Tihomir DODEV, Enfield (GB); Jenny MCINTOSH, Epsom (GB)

(21) Appl. No.: 17/772,806

(22) PCT Filed: Oct. 30, 2020
(86) PCT No.: PCT/GB2020/052760

§ 371 Date: Apr. 28, 2022

(30) Foreign Application Priority Data
Nov. 1, 2019 (GB) 1915953.2
Nov. 1, 2019 (GB) 1915955.7
Nov. 1, 2019 (GB) 1915956.5
Dec. 6, 2019 (GB) 1917925.8
Dec. 6, 2019 (GB) 1917926.6
Dec. 6, 2019 (GB) 1917927.4
Apr. 28, 2020 (GB) 2006250.1

(51) Int. Cl. C07K 14/755; A61K 48/00; C12N 15/86

(52) U.S. Cl. CPC C07K 14/755; A61K 48/0025; C12N 15/86; C12N 2750/14123; C12N 2750/14142

(57) ABSTRACT

The present invention provides Factor VIII polypeptides which comprise one or more substitution mutations compared to a corresponding wild-type Factor VIII, wherein the one or more substitution mutations are located at an inter-domain interface between two domains of the Factor VIII polypeptide. Also provided are polynucleotides comprising a Factor VIII nucleotide sequence encoding a Factor VIII polypeptide of the invention, recombinant AAV constructs comprising such polynucleotides, AAV viral particles comprising such recombinant AAV constructs, compositions comprising the Factor VIII polypeptide, polynucleotide, recombinant AAV construct, or AAV viral particle of the invention, and the use of the Factor VIII polypeptides, polynucleotides, recombinant AAV constructs, AAV viral particles and compositions of the invention in therapy.

### FIELD OF THE INVENTION

The present invention relates to a Factor VIII (FVIII) polypeptide, a polynucleotide comprising a Factor VIII nucleotide sequence, and a recombinant AAV construct. The invention further relates to an AAV viral particle comprising the recombinant AAV construct of the invention, and a composition comprising the Factor VIII polypeptide, polynucleotide, recombinant AAV construct or AAV viral particle of the invention. The invention also relates to methods of using, and uses of, the Factor VIII polypeptide, polynucleotide, recombinant AAV construct, AAV viral particle and/or composition of the invention.

### BACKGROUND OF THE INVENTION

Haemophilia A is a bleeding disorder caused by a deficiency of blood clotting Factor VIII. It affects 1:4,000 to 1:5,000 live male births worldwide. The majority of cases are inherited as an X-linked recessive trait. Current treatment involves frequent intravenous injections (2-3 times per week) of Factor VIII protein. This treatment is highly effective at arresting bleeding but it is not curative and is extremely expensive (£150,000/patient/year), thus making it unaffordable by the majority of haemophilia A patients in the world. Gene therapy for haemophilia A offers the potential for a cure through persistent, endogenous production of Factor VIII following the transfer of a functioning copy of the Factor VIII gene to an affected patient.

Factor VIII consists of six domains, namely A1-A2-B-A3-C1-C2. Factor VIII circulates in the bloodstream in an inactive form whilst bound to von Willebrand factor. In response to injury, Factor VIII is activated (often then referred to as Factor VIIIa) and separates from von Willebrand factor. Factor VIIIa interacts with Factor IXa in the coagulation cascade. In particular, Factor FVIIIa is a cofactor for Factor IXa in the activation of Factor X.

### SUMMARY OF THE INVENTION

The present invention relates to Factor VIII polypeptides which comprise particular amino acid substitutions (substitution mutations). Factor VIII is expressed and circulates in an inactive form as a heterodimeric complex consisting of the A1-A2-B domains, and A3-C1-C2 domains. Factor VIII is activated by proteolytic cleavage by thrombin. Following thrombin cleavage, Factor VIII forms a heterotrimeric complex consisting of the A1 domain, A2 domain, and A3-C1-C2 domains and undergoes a conformational change, which allows binding to Factor IXa and activation of Factor X. Following activation, Factor VIIIa may undergo further proteolysis, and/or the respective components of the heterotrimeric complex may dissociate from one-another, thereby inactivating Factor VIIIa. The present inventors have identified substitutions of certain amino acids situated at the interfaces of domains of Factor VIII which increase the specific activity of the Factor VIII polypeptide. Increasing the specific activity of the Factor VIII polypeptide, means that a lower amount of Factor VIII polypeptide may be required in order for coagulation to occur.

In a first aspect, there is provided a Factor VIII polypeptide comprising a Factor VIII amino acid sequence, wherein the FVIII amino acid sequence comprises one or more substitution mutations at an inter-domain interface selected from the group consisting of:
a. the A1/A3 domain interface;
b. the A2/A3 domain interface; or
c. the A1/C2 domain interface,
   wherein:
(i) the one or more substitution mutations comprises substitution of an amino acid with a more hydrophobic amino acid; or
(ii) the one or more substitution mutations comprises substitution of a pair of amino acids in the respective domains with cysteine residues;
   and wherein the Factor VIII polypeptide has higher specific activity than a reference wild-type Factor VIII polypeptide.

In a second aspect, there is provided a Factor VIII polypeptide comprising a Factor VIII amino acid sequence, wherein the Factor VIII amino acid sequence comprises one or more substitution mutations at an inter-domain interface selected from the group consisting of:
a. the A1/A3 domain interface;
b. the A2/A3 domain interface; or
c. the A1/C2 domain interface,
   wherein:
(i) the one or more substitution mutations comprises substitution of an amino acid with a more hydrophobic amino acid; or
(ii) the one or more substitution mutations comprises substitution of a pair of amino acids in respective domains with cysteine residues;
   and wherein the Factor VIII polypeptide has higher stability than a reference wild-type Factor VIII polypeptide.

In a third aspect, there is provided a Factor VIII polypeptide comprising a Factor VIII amino acid sequence, wherein the Factor VIII amino acid sequence comprises one or more substitution mutations at an inter-domain interface selected from the group consisting of:
a. the A1/A3 domain interface;
b. the A2/A3 domain interface; or
c. the A1/C2 domain interface,
   wherein:
(i) the one or more substitution mutations comprises substitution of an amino acid with a more hydrophobic amino acid; or
(ii) the one or more substitution mutations comprises substitution of a pair of amino acids in respective domains with cysteine residues;
   and wherein the Factor VIII polypeptide is expressed at a higher level in a host cell than a reference wild-type Factor VIII polypeptide.

In an fourth aspect, there is provided a Factor VIII polypeptide comprising a Factor VIII amino acid sequence, wherein the Factor VIII amino acid sequence comprises one or more substitution mutations selected from the group consisting of:
a. a substitution of an amino acid corresponding to M662 or H693 of SEQ ID NO: 1; or
b. a substitution of a pair of amino acids comprising a first amino acid and a second amino acid with cysteine residues, wherein:
   1. the first amino acid corresponds to M147, S149 or S289 of SEQ ID NO: 1 and the second amino acid corresponds to E1969, E1970 or N1977 of SEQ ID NO: 1;
   2. the first amino acid corresponds to T667, T669, N684, L687, I689, S695 or F697 of SEQ ID NO: 1 and the second amino acid corresponds to S1791, G1799, A1800, R1803, E1844, S1949, G1981, V1982, or Y1979 of SEQ ID NO: 1; or
   3. the first amino acid corresponds to A108, T118 or V137 of SEQ ID NO: 1 and the second amino acid corresponds to N2172, Q2329 or Y2332 of SEQ ID NO: 1.

In a fifth aspect, there is provided a polynucleotide comprising a Factor VIII nucleotide sequence, wherein the Factor VIII nucleotide sequence encodes a Factor VIII polypeptide of the invention.

In a sixth aspect, there is provided a recombinant AAV construct which comprises a polynucleotide comprising a Factor VIII nucleotide sequence, wherein the Factor VIII nucleotide sequence encodes a Factor VIII polypeptide of the invention.

In a seventh aspect, there is provided an AAV viral particle comprising the recombinant AAV construct of the invention.

In an eighth aspect, there is provided a composition comprising a Factor VIII polypeptide, polynucleotide, recombinant AAV construct or AAV viral particle of the invention and a pharmaceutically acceptable excipient.

In a ninth aspect, there is provided a Factor VIII polypeptide, polynucleotide, recombinant AAV construct, AAV viral particle or composition of the invention for use in a method of treatment.

In a tenth aspect, there is provided a method of treatment comprising administering an effective amount of the Factor VIII polypeptide, polynucleotide, recombinant AAV construct, AAV viral particle or composition of the invention to a patient.

In an eleventh aspect, there is provided a use of the Factor VIII polypeptide, polynucleotide, recombinant AAV construct, AAV viral particle or composition of the invention in the manufacture of a medicament for use in a method of treatment.

### DESCRIPTION OF THE FIGURES

FIG. 1 shows the Wimley-White hydrophobicity scale for the free energy (ΔG) transition of an amino acid from an aqueous phase to a non-aqueous phase (octanol). A more negative ΔG value is associated with a more favourable transition from the aqueous phase into the non-aqueous phase, and denotes a more hydrophobic amino acid.

FIG. 2 shows the fold-change in SA (specific activity), relative to the FVIII-SQ (‘95’) control lacking any substitution mutations, for several different amino-acid substitution mutation variants, including a number of alternative substituted residues for each variant. Variant 65 (H693W) exhibits an elevation in SA relative to 95.

FIG. 3 shows the fold-change in SA, relative to the FVIII-SQ (‘95’) control lacking any substitution mutations, for several different double cysteine substitution mutation variants. A number of variants exhibit an elevation in SA relative to 95.

FIG. 4 shows the Factor VIII specific activity of a series of Factor VIII polypeptides comprising substitution mutations. The effect of the substitution mutations on specific activity was assessed in ‘SQ’ and ‘96-106’ Factor VIII polypeptides, relative to a Factor VIII-SQ control which did not comprise any substitution mutations. Error bars represent the standard deviation from sample duplicates in the ELISA and activity assays.

FIG. 5 shows Factor VIII specific activity in plasma of C7BL/6 FVIII knockout mice intravenously injected with 2×10¹² vg/kg of AAV8 viral particles made from the constructs FRE72-SP5-FVIIICo19 (26-96-106)-SpA (SEQ ID NO:27); FRE72-SP5-FVIIICo19-SQ-SpA (SEQ ID NO:32); or a comparator construct comprising a codon-optimised FVIII-SQ-encoding sequence with native FVIII signal peptide, SpA and a liver-specific promoter (SEQ ID NO:34), six weeks post-injection. **P=0.0038. The left hand panel shows the specific activity of FRE72-SP5-FVIIICo19 (26-96-106)-SpA (left hand column) and the comparator construct (middle column) relative to a naïve control; the right hand panel shows the specific activity of FRE72-SP5-FVIIICo19-SQ-SpA (left hand column) and the comparator construct (right hand column).

### DESCRIPTION OF THE SEQUENCE LISTING

| SEQ ID | |
|---|---|
| NO: | DESCRIPTION |
| 1 | Wild-type FVIII amino acid sequence without signal peptide |
| 2 | Wild-type nucleotide sequence encoding FVIII without |
| | signal peptide |
| 3 | FVIII SQ amino acid sequence |
| 4 | Codop19 (Co19) nucleotide sequence encoding FVIII SQ |
| | nucleotide sequence |
| 5 | FVIII RE amino acid sequence |
| 6 | Codop19 (Co19) nucleotide sequence encoding FVIII |
| | polypeptide 96-106 |
| 7 | FVIII polypeptide 96-106 amino acid sequence |
| 8 | FVIII polypeptide 99-106 amino acid sequence |
| 9 | FVIII polypeptide 96-109 amino acid sequence |
| 10 | FVIII polypeptide 99-109 amino acid sequence |
| 11 | FVIII polypeptide 96-107 amino acid sequence |
| 12 | FVIII polypeptide 107-117 amino acid sequence |
| 13 | FRE72 transcription regulatory element (119 bp) |
| 14 | HLP2 transcription regulatory element (335 bp) |
| 15 | Amino acid sequence of native FVIII signal peptide |
| 16 | Wild-type nucleotide sequence encoding native FVIII |
| | signal peptide |
| 17 | Codon-optimised (Co19) nucleotide sequence encoding |
| | native FVIII signal peptide |
| 18 | Amino acid sequence of SP5 |
| 19 | Wild-type nucleotide sequence encoding SP5 |
| 20 | Amino acid sequence of SP10 |
| 21 | Wild-type nucleotide sequence encoding SP10 |
| 22 | PolyA sequence (SpA; 49 bp) |
| 23 | AAV2 5′ ITR sequence |
| 24 | AAV2 3′ ITR sequence |
| 25 | Amino acid sequence of AAV capsid |
| 26 | Amino acid sequence of AAV5 capsid |
| 27 | FVIII AAV construct (26-96-106) |
| 28 | FVIII 26-96-106 amino acid sequence |
| 29 | Codop19 (Co19) nucleotide sequence encoding 26-96-106 |
| 30 | FVIII 26-SQ amino acid sequence |
| 31 | Codop19 (Co19) nucleotide sequence encoding FVIII |
| | polypeptide 26 SQ |
| 32 | FVIII AAV construct (SQ) |
| 33 | FVIII AAV construct (26-SQ) |
| 34 | FVIII AAV construct (SEQ ID NO: 1 from WO 2017/053677) |
| 35 | FVIII 65-96-106 amino acid sequence |
| 36 | FVIII 26-65-96-106 amino acid sequence |
| 37 | FVIII 12SS-96-106 amino acid sequence |
| 38 | FVIII 28SS-96-106 amino acid sequence |
| 39 | FVIII 31SS-96-106 amino acid sequence |
| 40 | FVIII 65-SQ amino acid sequence |
| 41 | FVIII 26-65-SQ amino acid sequence |
| 42 | FVIII 12SS-SQ amino acid sequence |
| 43 | FVIII 28SS-SQ amino acid sequence |
| 44 | FVIII 31SS-SQ amino acid sequence |
| 45 | Amino acids 656-667 of SEQ ID NO: 1 |
| 46 | Amino acids 1823-1834 of SEQ ID NO: 1 |
| 47 | Amino acid sequence of LK03 capsid |
| 48 | Amino acid sequence of AAV6 capsid |

### DETAILED DESCRIPTION

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “a Factor VIII polypeptide comprising a Factor VIII amino acid sequence” should be interpreted to mean that the Factor VIII polypeptide has a Factor VIII amino acid sequence, but the Factor VIII polypeptide may contain further amino acids. Similarly, the phrase “a polynucleotide comprising a Factor VIII nucleotide sequence” refers to a polynucleotide that has a Factor VIII nucleotide sequence, but the polynucleotide may contain additional nucleotides.

In some embodiments of the invention, the word “comprising” is replaced with the phrase “consisting essentially of”. The term “consisting essentially of” means that specific further components can be present, namely those not materially affecting the essential characteristics of the subject matter.

In some embodiments of the invention, the word “comprising” is replaced with the phrase “consisting of”. The term “consisting of” is intended to be limiting. For example, the phrase “a Factor VIII polypeptide consisting of a Factor VIII amino acid sequence” should be interpreted to mean that the Factor VIII polypeptide has a Factor VIII amino acid sequence and no additional amino acids. Similarly, the phrase “a polynucleotide consisting of a Factor VIII nucleotide sequence” should be understood to mean that the polynucleotide has a Factor VIII nucleotide sequence and no additional nucleotides.

In some embodiments of the invention, the word “have” can be replaced with the word “comprise” or the phrase “consist of”.

The terms “protein” and “polypeptide” are used interchangeably herein, and are intended to refer to a polymeric chain of amino acids of any length.

The terms “nucleic acid molecule” “nucleic acid sequence”, “polynucleotide” and “nucleotide sequence” are used interchangeably herein, and are intended to refer to a polymeric chain of nucleotides of any length.

The terms “substitution mutation” and “amino acid substitution” are used interchangeably herein, and are intended to mean the substitution of one amino acid in an amino acid sequence with a different amino acid. In the phrases “substitution of” amino acid X, or “amino acid X that is (to be) substituted” amino acid X is the original or native amino acid that is present within an amino acid sequence and that is to be replaced. For example, substitution of methionine means that a native methionine amino acid is replaced by another amino acid. In the phrase “substitution with” amino acid Y, amino acid Y is the different amino acid which replaces the original or native amino acid in an amino acid sequence. For example, substitution with methionine refers to replacement of a native (non-methionine) amino acid with methionine. The standard shorthand nomenclature used to define a substitution mutation lists the original or native amino acid at a position within an amino acid sequence that is to be substituted, and the amino acid which replaces the original or native amino acid. For example, a Factor VIII amino acid sequence comprising the substitution mutation M662W refers to a Factor VIII amino acid sequence which comprises a substitution of the methionine residue at a position corresponding to position 662 with a tryptophan residue (i.e. which comprises a tryptophan residue at position 662).

The term “conservative substitution” refers to a substitution mutation in which an amino acid is substituted with another amino acid which has similar biochemical properties, such as size, charge or hydrophobicity. Amino acids may be categorised into groups on the basis of the structure of their side chains: aliphatic (glycine, alanine, valine, leucine, isoleucine); hydroxyl/sulphur-containing (serine, threonine, cysteine, methionine); cyclic (proline); aromatic (phenylalanine, tyrosine, tryptophan); basic (histidine, lysine, arginine); acidic (aspartic acid, glutamic acid); and acid amine (asparagine, glutamine). A “conservative substitution” thus refers to a substitution mutation in which an amino acid is substituted with another amino acid in the same group. Conversely, the term “non-conservative substitution” refers to a substitution in which an amino acid is substituted with another amino acid which has different biochemical properties, i.e. a substitution mutation in which an amino acid is substituted with an amino acid in another group. For example, substitution of aspartic acid with glutamic acid may be considered to be a “conservative substitution”, whilst substitution of aspartic acid with valine may be considered to be a “non-conservative” substitution.

The terms “wild-type” and “native” are used interchangeably herein, and are intended to describe something which is naturally occurring. For example, a “wild-type Factor VIII amino acid sequence” is an amino acid Factor VIII sequence which occurs in nature.

The terms “B domain” and “beta domain” are used interchangeably herein in relation to the B domain of Factor VIII. The B domain of wild-type Factor VIII consists of the region which corresponds to amino acids 741-1648 of SEQ ID NO:1.

The terms “AAV viral particle” and “AAV vector” are used interchangeably herein.

The term “around” used in the context of describing the length of nucleotide or amino acid sequences indicates that a sequence may comprise or consist of a defined number of nucleotides or amino acids, plus or minus 10%, more particularly plus or minus 5%, or more particularly plus or minus a single integer. For example, reference to an amino acid sequence of “around” 45 amino acids in length may refer to an amino acid sequence of 41-49 amino acids, more particularly 43-47 amino acids, and more particularly 44-46 amino acids in length.

For the purpose of this invention, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide or amino acid residues at each position are then compared. When a position in the first sequence is occupied by the same nucleotide or amino acid as the corresponding position in the second sequence, then the nucleotides or amino acids are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 3, SEQ ID NO: 3 would be the reference sequence. To assess whether a sequence is at least 95% identical to SEQ ID NO: 3 (an example of a reference sequence), the skilled person would carry out an alignment over the length of SEQ ID NO: 3, and identify how many positions in the test sequence were identical to those of SEQ ID NO: 3. If at least 95% of the positions are identical, the test sequence is at least 95% identical to SEQ ID NO: 3. If the sequence is shorter than SEQ ID NO: 3, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

The singular forms “a”, “an”, and “the” include plural referents unless the content clearly dictates otherwise. Thus, for example, reference to “an amino acid” includes two or more instances or versions of such amino acids.

All publications, patents and patent applications cited herein, whether supra or infra, are hereby incorporated by reference in their entirety.

### A Gain of Function Mutation

It is believed that the dissociation of the respective components of the activated Factor VIII heterotrimeric complex (and in particular, dissociation of the A2 domain from the A1 domain and A3-C1-C2 domains) is one of the principal mechanisms for the loss of Factor VIII activity following activation of the clotting cascade. Without wishing to be bound by theory, it is believed that the instant amino acid substitution mutations may serve to prevent or delay dissociation of the respective components of the Factor VIII heterotrimeric complex following activation of the Factor VIII polypeptide, thereby extending the time for which the active FVIIIa is intact, and thus active.

The Factor VIII polypeptides of the invention comprise one or more substitution mutations, compared to a corresponding wild-type Factor VIII amino acid sequence. More particularly, the one or more substitution mutation is located at an inter-domain interface between two domains of the Factor VIII polypeptide. In particular, the one or more substitution mutations are located at the inter-domain interface selected from the A1/A3 domain interface, the A2/A3 domain interface, or the A1/C2 domain interface. The Factor VIII amino acid sequence therefore comprises a substitution mutation in the A1, A2, A3 or C2 domain. Three-dimensional crystal structures of Factor VIII are available (for example, the PDB accession numbers 2RZE or 4BDV); and it would therefore be routine to identify amino acids within these domains, and more particularly amino acids which are situated at specified inter-domain interfaces, which may be substituted. With regard to wild-type Factor VIII, the A1 domain consists of amino acids corresponding to positions 1-329 of SEQ ID NO:1; the A2 domain consists of amino acids corresponding to positions 380-711 of SEQ ID NO:1; the A3 domain consists of amino acids corresponding to positions 1694-2021 of SEQ ID NO:1; the C1 domain consists of amino acids corresponding to positions 2021-2169 of SEQ ID NO:1; and the C2 domain consists of amino acids corresponding to positions 2174-2332 of SEQ ID NO:1. The last six amino acids (corresponding to positions 2327 to 2332 of SEQ ID NO: 1) may not be considered part of the C2 domain. Thus, optionally, the C2 domain consists of the amino acids corresponding to positions 2174-2326 of SEQ ID NO: 1.

In particular embodiments, a Factor VIII polypeptide comprising a Factor VIII amino acid sequence comprising one or more substitution mutations at the A1/A3 domain interface, the A2/A3 domain interface or the A1/C2 domain interface as disclosed herein may have higher specific activity than a reference wild-type Factor VIII polypeptide. Exemplary substitution mutations which increase the specific activity of a Factor VIII polypeptide are disclosed herein.

Factor VIII is a cofactor for Factor X in the clotting cascade, and once activated acts in combination with activated Factor IX to activate Factor X. Factor VIII does not independently possess enzymatic activity as such. Thus, reference to the activity or specific activity of a Factor VIII polypeptide herein refers to the observed activity or specific activity in a functional assay for determining the activity of Factor X, in which Factor VIII is able to act as a cofactor for Factor X in combination with Factor IX (i.e. Factor VIII cofactor activity (FVIII:C)). Similarly, reference to a Factor VIII polypeptide having higher activity or specific activity than a reference Factor VIII polypeptide (such as a wild-type Factor VIII polypeptide) refers to the observed activity or specific activity of Factor X in a functional assay being increased for said Factor VIII polypeptide, relative to the observed activity or specific activity of Factor X in said assay for a reference Factor VIII polypeptide.

For the purposes of determining whether a Factor VIII polypeptide has increased specific activity relative to a reference Factor VIII polypeptide such as a reference wild-type Factor VIII polypeptide, the activity of the Factor VIII polypeptide may be measured by a two stage chromogenic Factor Xa assay. For example, a suitable chromogenic assay is as follows. The Factor VIII polypeptide is mixed with human Factor X polypeptide and Factor IXa polypeptide, thrombin, phospholipids and calcium. The thrombin activates the Factor VIII polypeptide to form Factor VIIIa polypeptide. The thrombin-activated Factor VIII polypeptide forms an enzymatic complex with Factor IXa polypeptide, phospholipids and calcium, which enzymatic complex can catalyse the conversion of Factor X polypeptide to Factor Xa polypeptide. The activity of the Factor Xa polypeptide can catalyse cleavage of a chromogenic substrate (e.g. SXa-11) to produce pNA. The level of pNA generated can be measured by determining colour development at 405 nm (e.g. measured by absorbance). Factor X polypeptide, and therefore Factor Xa polypeptide, is provided in excess. Therefore the limiting factor is Factor VIIIa polypeptide. Thus, the level of pNA generated is proportional to the amount of the Factor Xa polypeptide generated by Factor FVIIIa polypeptide in the sample, which is proportional to the activity of Factor FVIIIa polypeptide in the sample. The activity of Factor FVIIIa polypeptide in the sample is a measure of the cofactor activity of the Factor FVIII polypeptide in the sample.

For example, a suitable chromogenic assay is the BIOPHEN FVIII:C assay (Ref: 221406) manufactured by HYPHEN BioMed as used in the Examples. The activity of the Factor VIII polypeptide may be measured using the BIOPHEN FVIII:C assay. More particularly, the activity of the Factor VIII polypeptide may be measured using the BIOPHEN FVIII:C assay according to the protocol described below in Example 1.

For the purposes of the present application, the term “specific activity” refers to the activity (e.g. clotting activity or intrinsic enzyme activity) per unit (e.g. per μg, per IU, or per antigen level as % of level in normal human plasma) of Factor VIII polypeptide such that the activity is ‘normalised’ to take account of the amount or concentration of Factor VIII polypeptide in the sample. (Note that, typically, pooled healthy human plasma has a Factor VIII concentration of 0.2m/ml.) This can be done by measuring the concentration of the Factor VIII polypeptide in the sample, for example by using a standard ELISA assay, such as the assay described in Example 1, and dividing the activity by the Factor VIII concentration. A chromogenic assay may be used to measure “specific activity”. A chromogenic assay may be used to measure “specific activity” by calculating activity and dividing by the concentration of the Factor VIII polypeptide in the sample. The chromogenic assay may be any one of the chromogenic assays described herein.

In an example of an ELISA assay, an antibody that binds to the Factor VIII polypeptide could be bound to a plate. The sample, comprising the Factor VIII polypeptide at unknown concentration, could be passed over the plate. A second detection antibody that binds to the Factor VIII polypeptide could be applied to the plate, and any excess washed off. The detection antibody that remains (i.e. is not washed off) will be bound to the Factor VIII polypeptide. The detection antibody could be linked to an enzyme such as horse radish peroxidase. The level of detection antibody that binds to the Factor VIII polypeptide on the plate could be measured by measuring the amount of the detection antibody. For example, if the detection antibody is linked to horse radish peroxidase, the horse radish peroxidase can catalyse the production of a blue reaction product from a substrate such as TMB (3,3′,5,5′-tetramethylbenzidine), and the level of the blue product can be detected by absorbance at 450 nm. The level of the blue product is proportional to the amount of detection antibody that remained after the washing step, which is proportional to the amount of the Factor VIII polypeptide in the sample. Alternatively, for example when using purified protein, the amount or concentration of Factor VIII polypeptide may be determined spectrophotometrically.

In certain embodiments, the Factor VIII polypeptide is purified, and the specific activity is measured by a chromogenic assay carried out on the purified Factor VIII polypeptide. In some embodiments, the specific activity of the Factor VIII polypeptide is measured by generating an AAV particle comprising a polynucleotide encoding the Factor VIII polypeptide, injecting mice with the AAV particle, and detecting the specific activity in plasma from the mice using a chromogenic assay. In some embodiments, the specific activity of the Factor VIII polypeptide is measured by providing cells stably expressing a polynucleotide encoding the Factor VIII polypeptide, harvesting Factor VIII polypeptide from the cells and/or culture medium, and measuring the specific activity of the Factor VIII polypeptide using a chromogenic assay.

The Factor VIII polypeptide may have a specific activity which is higher than the specific activity of a reference Factor VIII polypeptide (such as a reference wild-type Factor VIII polypeptide). The Factor VIII polypeptide may have a specific activity which is at least 1.1 fold, at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of a reference Factor VIII polypeptide. The Factor VIII polypeptide may have a specific activity which is between 1.2 fold and 5.5 fold, or between 1.5 fold and 5 fold, higher than the specific activity of a reference Factor VIII polypeptide. When referring to fold changes of activity, the term “between” includes the specified values. Thus, for example, “between 1.2 fold and 5.5 fold” includes the values 1.2 and 5.5.

In further embodiments, a Factor VIII polypeptide comprising a Factor VIII amino acid sequence comprising one or more substitution mutations at the A1/A3 domain interface, the A2/A3 domain interface or the A1/C2 domain interface as disclosed herein may have higher stability than a reference wild-type Factor VIII polypeptide.

A Factor VIII polypeptide having higher stability than a reference Factor VIII polypeptide retains a higher proportion of its Factor VIII activity over time than a reference Factor VIII polypeptide. The Factor VIII polypeptide of the invention may have a higher stability than a reference Factor VIII polypeptide prior to being activated, i.e. the Factor VIII polypeptide may have higher stability in its inactive form than a reference Factor VIII polypeptide in its inactive form. Put another way, an inactive Factor VIII polypeptide of the invention may have a higher stability than an inactive reference Factor VIII polypeptide. Alternatively or additionally, the Factor VIII polypeptide of the invention may have a higher stability than a reference Factor VIII polypeptide when activated. Put another way, an active Factor VIII polypeptide of the invention may have a higher stability than an active reference Factor VIII polypeptide.

A Factor VIII polypeptide may have higher stability than a reference Factor VIII polypeptide prior to activation. A higher proportion of a Factor VIII polypeptide having a higher stability prior to activation than a reference Factor VIII polypeptide may remain capable of being activated over time than a reference Factor VIII polypeptide. Stability of a Factor VIII polypeptide prior to activation may be determined by measuring residual Factor VIII activity in a sample over time. Aliquots of a sample containing inactivated Factor VIII polypeptide may be removed at suitable time points, and Factor VIII polypeptide activity may be determined by activating the Factor VIII polypeptide in an aliquot and performing a two stage Factor X chromogenic assay as described herein. Activity at a given time point may then be compared to the initial Factor VIII activity of the sample (i.e. by determining Factor VIII activity at an initial time point), and the residual Factor VIII activity may be calculated as a percentage of the initial Factor VIII activity. Thus, when activated, the Factor VIII polypeptide having higher stability prior to activation than a reference Factor VIII polypeptide will have a higher residual activity (i.e. as a percentage of the initial Factor VIII polypeptide activity) than the reference Factor VIII polypeptide.

Optionally, residual activity of an inactive Factor VIII polypeptide in a sample may be measured over the course of five, ten, fifteen, twenty, twenty-five, thirty, forty-five or sixty minutes, or over the course of 2 hours, 3 hours, 4 hours, 5 hours, 6 hours, 7 hours, 8 hours, 9 hours, 10 hours, 11 hours, 12 hours, 18 hours or 24 hours, or over the course of 2 days, 3 days, 4 days, 5 days, 6 days or 7 days. Aliquots of a sample may be taken at a series of time points, e.g. two or more, three or more, four or more, five or more or six or more time points, in addition to taking an aliquot of the sample at an initial time point and Factor VIII activity in each aliquot may be determined. Comparing Factor VIII activity at each time point with Factor VIII activity at the initial time point allows residual Factor VIII activity to be determined. Optionally, specific activity may be determined at each time point.

A Factor VIII polypeptide may have higher stability than a reference Factor VIII polypeptide when activated. A Factor VIII polypeptide having a higher stability when activated than a reference FVIII polypeptide may retain a higher proportion of its Factor VIII activity over time than a reference Factor VIII polypeptide following activation. Stability of a Factor VIII polypeptide following activation may be determined by measuring Factor VIII polypeptide activity in a sample over time, i.e. in a FVIIIa activity decay assay. A Factor VIII polypeptide may be activated using thrombin for 1 minute at 23° C. and immediately quenched using hirudin to inactivate thrombin. Aliquots may be removed at suitable time points and the activity may be determined using a Factor X chromogenic assay.

Optionally, activity of an active Factor VIII polypeptide in a sample may be measured over the course of five, ten, fifteen, twenty, twenty-five or thirty minutes following activation. Activity may be determined at a series of time points, e.g. two or more, three or more, four or more, five or more or six or more time points following activation, in addition to determining the activity of Factor VIII in a sample following activation (e.g. immediately following activation). By way of representative example, the activity of Factor VIII in a sample may be determined immediately following activation, and further measurements may be taken at a series of up to six time points within twenty minutes of activation. Optionally, specific activity may be determined at each time point.

A Factor VIII polypeptide having higher stability than a reference Factor VIII polypeptide may have a longer half-life relative to a reference Factor VIII polypeptide. Optionally, the Factor VIII polypeptide has a longer half-life than a reference Factor VIII polypeptide prior to activation. Optionally, the Factor VIII polypeptide has a longer half-life than a reference Factor VIII when activated. Optionally, the Factor VIII polypeptide has a half-life which is at least 1.1, at least 1.2, at least 1.5, at least 1.7, at least 1.8, at least 2, at least 2.2, at least 2.5, at least 2.8 or at least 3 times the half-life of a reference wild-type Factor VIII polypeptide. Optionally, the Factor VIII polypeptide has a half-life when activated which is at least 1.1, at least 1.2, at least 1.5, at least 1.7, at least 1.8, at least 2, at least 2.2, at least 2.5, at least 2.8 or at least 3 times the half-life of a reference Factor VIII polypeptide when activated. Optionally, the Factor VIII polypeptide has a half-life when activated with is between 1.1 and 3, between 1.2 and 3, between 1.5 and 3, between 1.7 and 3, between 1.8 and 3, between 2 and 3, between 2.2 and 3, between 2.5 and 3, or between 2.8 and 3 times the half-life of a reference Factor VIII polypeptide when activated.

Optionally, the Factor VIII polypeptide may have higher stability in a liquid (i.e. when in solution). Optionally, the Factor VIII polypeptide has a longer half-life in a liquid. Optionally, the liquid is conditioned medium, such as conditioned medium in which a host cell expressing a Factor VIII polypeptide is cultured. Optionally, the liquid is a biological sample. A biological sample may be blood, serum or plasma. Optionally, the Factor VIII polypeptide may have higher stability in plasma. Optionally, the Factor VIII polypeptide may have a longer half-life in plasma.

In yet further embodiments, a Factor VIII polypeptide comprising a Factor VIII amino acid sequence comprising a substitution of one or more amino acids at the A1/A3 domain interface, the A2/A3 domain interface or the A1/C2 domain interface as disclosed herein may be expressed at a higher level in a host cell than a reference Factor VIII polypeptide. For example, a Factor VIII polypeptide comprising the substitution may be expressed at least 1.1 fold, at least 1.2 fold, at least 1.5 fold, at least 1.8 fold, at least 2 fold, at least 2.5 fold, at least 3 fold, at least 3.5 fold, at least 4 fold, at least 4.5 fold or at least 5 fold higher compared to a reference Factor VIII polypeptide. Optionally, the Factor VIII polypeptide comprising the substitution may be expressed between 1.1 fold and 5 fold, between 1.2 fold and 5 fold, between 1.5 fold and 5 fold, between 1.8 fold and 5 fold, between 2 fold and 5 fold, between 2.5 fold and 5 fold, between 3 fold and 5 fold, between 3.5 fold and 5 fold, between 4 fold and 5 fold or between 4.5 fold and 5 fold higher compared to a reference Factor VIII polypeptide. Optionally, the Factor VIII polypeptide is secreted by a host cell at a higher level than a reference Factor VIII polypeptide.

The level of expression of a Factor VIII polypeptide (and therefore whether the Factor VIII polypeptide is expressed at a higher level in a host cell than a referenced wild-type Factor VIII polypeptide) may typically be determined by measuring the level of Factor VIII polypeptide in a sample. The level of expression of a Factor VIII polypeptide of the invention in a host cell may be compared with the level of expression of a reference Factor VIII polypeptide in a host cell. This may be determined quantitatively. For example, the level of expression of a Factor VIII polypeptide may be determined by an ELISA assay as described above. Alternatively, the level of expression of a Factor VIII polypeptide may be determined semi-quantitatively, for example, by SDS-PAGE electrophoresis or by Western blot. In certain embodiments, expression in plasma can be determined by the Asserachrom assay as described in the Examples.

The level of expression of a Factor VIII polypeptide that is secreted by a host cell is typically determined, i.e. in contrast to the level of the Factor VIII polypeptide that is retained intracellularly by a host cell. This may be determined, for example, by separating cells (e.g. host cells) from a liquid containing the Factor VIII polypeptide (e.g. a biological sample, such as blood, serum or plasma, or culture medium such as conditioned medium in which a host cell expressing a Factor VIII polypeptide is cultured) and determining the level of expression of a Factor VIII polypeptide in the liquid. Cells may be separated, for example, by centrifugation or filtration, and/or the liquid may be decanted (e.g. by pipetting) from the cells. The level of expression of a Factor VIII polypeptide may be determined in a biological sample, such as blood, serum or plasma, or may be determined in a culture medium, such as conditioned medium in which a host cell expressing a Factor VIII polypeptide is cultured.

A host cell may be any eukaryotic host cell expressing a Factor VIII polypeptide (or a reference Factor VIII polypeptide). Optionally, the host cell may be an insect cell expressing a Factor VIII polypeptide. Typically, the host cell may be a mammalian host cell expressing a Factor VIII polypeptide. Mammalian host cells include human, dog, pig, mouse, hamster, or guinea pig cells. More particularly, the host cell may be a mammalian liver cell, and more particularly may be a human liver cell. Optionally, the host cell may be an Huh7 cell. Optionally, the host cell may be a host cell within an organism. Optionally, expression may be in vivo expression. Optionally, expression may be in vivo expression and expression in plasma may be determined.

The Factor VIII polypeptides of the invention may have higher specific activity and/or stability and/or may be expressed at a higher level in a host cell than a reference Factor VIII polypeptide. Optionally, the reference Factor VIII polypeptide may be a wild-type Factor VIII polypeptide, i.e. the reference Factor VIII polypeptide may be a reference wild-type Factor VIII polypeptide. The “reference wild-type Factor VIII polypeptide” may be any wild-type Factor FVIII polypeptide, such as the Factor VIII polypeptide of SEQ ID NO: 1. Optionally, the reference Factor VIII polypeptide is a beta domain deleted Factor VIII polypeptide. Optionally, the reference Factor VIII polypeptide has an amino acid sequence set forth in SEQ ID NOs: 3 or 5. Optionally, the reference Factor VIII polypeptide comprises the Factor VIII amino acid sequence of the Factor VIII polypeptide of the invention, but does not comprise the one or more substitution mutations.

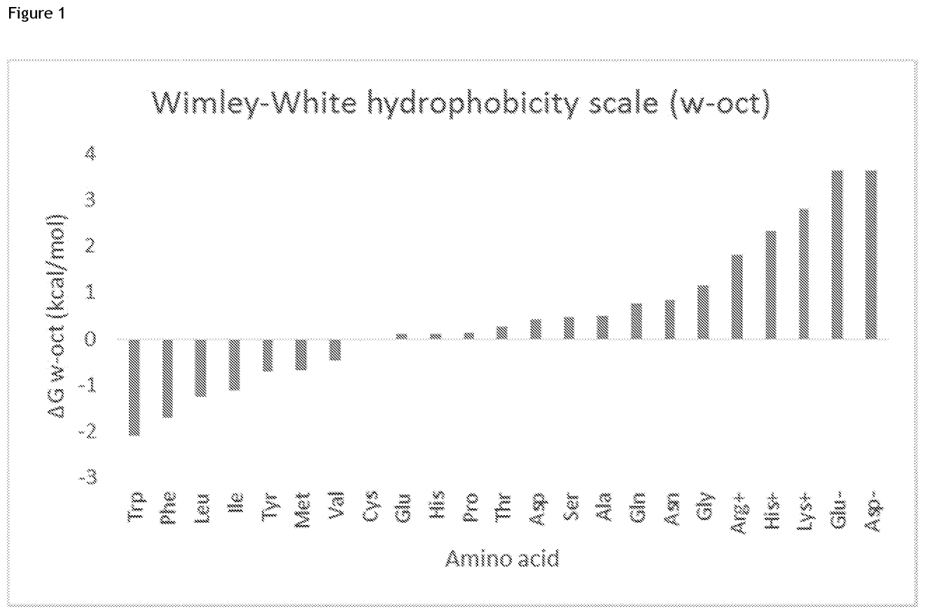

The one or more substitution mutations may comprise substitution of an amino acid at an inter-domain interface with a more hydrophobic amino acid. Biophysical studies have been performed to establish the hydrophobicity of the twenty naturally occurring amino acids, or more particularly, the relative hydrophobicity of the amino acids, and hydrophobicity scales list the hydropathy of each of the amino acids. One such example is the Wimley-White whole-residue hydrophobicity scale, which calculates the free energy of transfer of an amino acid from an aqueous phase to a non-aqueous phase (octanol). The term “a more hydrophobic amino acid” refers to an amino acid which has a more favourable (more negative ΔG) free energy value for the transition from an aqueous phase to octanol according to the Wimley-White hydrophobicity scale. The free energy for the aqueous phase to octanol transition is shown in Table 2 below, and is shown graphically in FIG. 1. Starting with the most hydrophobic amino acid, the Wimley-White hydrophobicity scale lists the hydrophobicity of amino acids in the following order: tryptophan, phenylalanine, leucine, isoleucine, tyrosine, methionine, valine, cysteine, glutamic acid (uncharged), histidine (uncharged), proline, threonine, aspartic acid (uncharged), serine, alanine, glutamine, asparagine, glycine, arginine (positively charged), histidine (positively charged), lysine (positively charged), glutamic acid (negatively charged), aspartic acid (negatively charged).

TABLE 2
Wimley-White whole residue hydrophobicity
scale for water to octanol transition.

| Amino | Octanol Scale |
|---|---|
| acid | ΔG w-oct (kcal/mol) |
| Trp | −2.09 |
| Phe | −1.71 |
| Leu | −1.25 |
| Ile | −1.12 |
| Tyr | −0.71 |
| Met | −0.67 |
| Val | −0.46 |
| Cys | −0.02 |
| Glu | 0.11 |
| His | 0.11 |
| Pro | 0.14 |
| Thr | 0.25 |
| Asp | 0.43 |
| Ser | 0.46 |
| Ala | 0.5 |
| Gln | 0.77 |
| Asn | 0.85 |
| Gly | 1.15 |
| Arg+ | 1.81 |
| His+ | 2.33 |
| Lys+ | 2.8 |
| Glu− | 3.63 |
| Asp− | 3.64 |

The substitution mutations of the invention may stabilise the interaction between two or more of the domains of the Factor VIII polypeptide. For example, the one or more substitution mutations at an inter-domain interface may stabilise the interaction between the respective domains of the Factor VIII polypeptide. In particular, one or more substitution mutations at the A1/A3 inter-domain interface may stabilise the interaction of the A1 and A3 domains, one or more substitution mutations at the A2/A3 inter-domain interface may stabilise the interaction of the A2 and A3 domains and one or more substitution mutations at the A1/C2 inter-domain interface may stabilise the interaction of the A1 and C2 domains. Optionally, the one or more substitution mutations of the invention may stabilise the interaction of the respective domains of the Factor VIII polypeptide when activated. In particular, the one or more substitution mutations of the invention may stabilise the interaction of the A1 and A3 domains, the A2 and A3 domains, and/or the A1 and C2 domains (i.e. of the respective domains) of the Factor VIII polypeptide when activated.

Optionally, the one or more substitution mutations of the invention may stabilise the interaction of the A1 domain, the A2 domain, and the A3-C1-C2 domains of the activated Factor VIII heterotrimeric complex. Optionally, the one or more substitution mutations may stabilise the interaction of the A2 domain with the A1 domain and A3-C1-C2 domains of the activated Factor VIII heterotrimeric complex. Optionally, the one or more substitution mutations may prevent or delay dissociation of the A2 domain from the A1 domain and A3-C1-C2 domains of the activated Factor VIII heterotrimeric complex.

The interaction of the A1 domain, A2 domain, and A3-C1-C2 domains of Factor VIII may be determined by surface plasmon resonance (SPR, or Biacore). Different components may be isolated separately, and one of the domains may be immobilised on the surface of an SPR chip; following immobilisation, the other domains may be injected into a flow cell and the binding and dissociation kinetics (kₒₙ and k_{off}) for the interaction of the domains may be monitored over time. Alternatively, inactive Factor VIII may be immobilised on an SPR chip and thrombin may be injected into a flow cell to activate Factor VIII; a drop in signal may be monitored over time, which represents the dissociation (loss of mass) of the respective components of the Factor VIII heterotrimeric complex (k_{off}). By either means, comparison with a reference Factor VIII polypeptide (e.g. a reference wild-type Factor VIII polypeptide) allows the stability of the interaction of the domains to be determined. The use of surface plasmon resonance to determine the stability of FVIIIa is described in Gale et al. 2006. Journal of Thrombosis and Haemostasis 4, 1315-1322.

Inter-domain interactions within a polypeptide are typically mediated by interactions between amino acid side chains in each of the respective domains. Interactions between the amino acid side-chains in respective domains may be non-covalent interactions. Alternatively, the amino acid side-chains in respective domains may form a covalent bond (e.g. a disulphide bond). The interaction between the domains of the Factor VIII polypeptide may therefore be stabilised (i.e. relative to a reference Factor VIII polypeptide) by amino acid substitution(s) (substitution mutations) which stabilise the interaction of the respective domains (e.g. the A1 and A3, A2 and A3 or A1 and C2 domains) of the Factor VIII polypeptide.

The side chains of aromatic amino acids phenylalanine, tyrosine, histidine and tryptophan may interact with one-another by pi-stacking interactions. In pi-stacking interactions, pairs of aromatic side-chains may typically align their respective aromatic rings in an off-centred parallel orientation. Alternatively, pairs of aromatic side-chains may align their respective aromatic rings in a T-shaped, perpendicular orientation to one-another. Optionally, the Factor VIII polypeptide may comprise a Factor VIII amino acid sequence which comprises one or more substitution mutations which increases pi-stacking interactions between amino acid side-chains of the respective domains.

The side chains of hydrophobic amino acids glycine, alanine, valine, isoleucine, leucine, phenylalanine, tryptophan, tyrosine and methionine typically cluster together within the hydrophobic core of a protein. Minimising the number of hydrophobic side chains exposed to water is a principal driving force behind protein folding, and hydrophobic packing contributes to stabilising protein structure. Conversely, the side chains of charged and polar amino acids are situated on the water-exposed surface of the protein where they interact with the surrounding water molecules. Optionally, the Factor VIII polypeptide may comprise a Factor VIII amino acid sequence which comprises one or more substitution mutations which increases hydrophobic packing between the amino acid side-chains of the respective domains (i.e. providing a more hydrophobic environment for the side chains of aliphatic/hydrophobic amino acids to interact with one another and more efficiently exclude water.

Optionally, the one or more substitution mutations comprises substitution of a charged or polar amino acid at an inter-domain interface with a hydrophobic amino acid. Optionally, the one or more substitution mutations comprises substitution of a charged or polar amino acid at an inter-domain interface with glycine, alanine, valine, isoleucine, leucine, phenylalanine, tryptophan, tyrosine or methionine. Optionally, the one or more substitution mutations comprises substitution of a hydrophobic amino acid at an inter-domain interface with a more hydrophobic amino acid.

Amino acid side chains in the respective domains may interact with one another in an unfavourable manner, due to the orientation of the respective domains relative to one-another. For example, amino acid side chains may be forced to adopt an energetically unfavourable conformation due to the orientation of the respective domains relative to one-another (steric clashing). Alternatively, an amino acid side chain may be positioned in proximity to a similarly charged amino acid side chain in another domain (an unfavourable electrostatic interaction).

Optionally, the one or more substitution mutations may eliminate unfavourable interactions between the amino acid side-chains of the respective domains. Optionally, the one or more substitution mutations may reduce steric clashing between amino acid side chains of the respective domains. Optionally, the one or more substitution mutations to reduce steric clashing may comprise substitution of a large hydrophobic amino acid with a smaller amino acid. Optionally, the one or more substitution mutations may comprise substitution of a large hydrophobic amino acid with isoleucine, leucine, valine, alanine or glycine. Optionally, the large hydrophobic amino acid is an aromatic amino acid. Optionally, the one or more substitution mutations may reduce unfavourable electrostatic interactions between amino acid side chains of the respective domains. Optionally, the one or more substitution mutations may comprise substitution of a positively charged amino acid. Optionally, the one or more substitution mutations may comprise substitution of a positively charged amino acid with a negatively charged amino acid. Optionally, the negatively charged amino acid is aspartic acid or glutamic acid. Optionally, the one or more substitution mutations may comprise substitution of a negatively charged amino acid. Optionally, the one or more substitution mutations may comprise substitution of a negatively charged amino acid with a positively charged amino acid. Optionally, the positively charged amino acid is arginine or lysine. Optionally, the one or more substitution mutations may comprise substitution of a charged amino acid with an uncharged amino acid. Optionally, the uncharged amino acid may be a polar amino acid. Optionally, the uncharged amino acid may be asparagine or glutamine. Optionally, the uncharged amino acid may be serine or threonine. Optionally, the uncharged amino acid may be valine, isoleucine, leucine, alanine or glycine.

Optionally, the one or more substitution mutation may comprise a substitution of one or more surface-inaccessible amino acids at an inter-domain interface. Three-dimensional crystal structures of Factor VIII are available (for example, the PDB accession numbers 2RZE or 4BDV); and it would therefore be routine to identify amino acids at an inter-domain interface which are surface-inaccessible. Optionally, the surface-inaccessible amino acid is surface-inaccessible in a Factor VIII polypeptide when activated.

The Factor VIII polypeptides of the invention may comprise a Factor VIII amino acid sequence which comprises one or more substitution mutations at an inter-domain interface selected from the A1/A3 interface, the A2/A3 interface, or the A1/C2 interface, wherein the amino acid which is substituted is methionine or histidine. Methionine or histidine residues at these inter-domain interfaces may be identified, for example, from a three-dimensional crystal structure of Factor VIII. Optionally, the methionine or histidine may be substituted with a more hydrophobic amino acid. Optionally, a methionine residue may be substituted with tyrosine, isoleucine, leucine, phenylalanine or tryptophan. Optionally, a histidine residue may be substituted with glutamic acid, cysteine, valine, methionine, tyrosine, isoleucine, leucine, phenylalanine or tryptophan. Optionally, the Factor VIII amino acid sequence may comprise one or more substitution mutations, wherein the amino acid which is substituted is the methionine residue corresponding to the amino acid at position 662 of SEQ ID NO: 1 (M662), or the histidine residue corresponding to the amino acid at position 693 of SEQ ID NO:1 (H693). Optionally, M662 may be substituted with alanine, cysteine, glutamic acid, glycine or serine. Optionally, H693 may be substituted with arginine. Optionally, M662 or H693 may be substituted with a more hydrophobic amino acid. Optionally, M662 may be substituted with tyrosine, isoleucine, leucine, phenylalanine or tryptophan. Optionally, H693 may be substituted with glutamic acid, cysteine, valine, methionine, tyrosine, isoleucine, leucine, phenylalanine or tryptophan.

It is within the capabilities of the person skilled in the art to determine the residue which “corresponds to” a particular amino acid of SEQ ID NO: 1. For example, the person skilled in the art merely needs to perform a sequence alignment of the wild-type Factor VIII amino acid sequence with SEQ ID NO: 1 using a suitable alignment algorithm such as that of Needleman and Wunsch described above, and determine which residue aligns with the amino acid of SEQ ID NO: 1.

Optionally, the one or more substitution mutations comprises substitution of an amino acid at an inter-domain interface with an aromatic amino acid. Optionally, the one or more substitution mutations comprises substitution of an amino acid at an inter-domain interface with phenylalanine, tyrosine, histidine or tryptophan. Optionally, the one or more substitution mutations comprises substitution of a methionine residue at an inter-domain interface with phenylalanine, tyrosine or tryptophan. Optionally, the one or more substitution mutations comprises substitution of a histidine residue at an inter-domain interface with phenylalanine, tyrosine or tryptophan. Optionally, the one or more substitution mutations comprises the M662F, M662W or M662Y substitution. Optionally, the one or more substitution mutations comprises the H693F, H693W or H693Y substitution. Optionally, the one or more substitution mutations comprises the M662W substitution. Optionally, the one or more substitution mutations comprises the H693W or H693Y substitution. Optionally, the one or more substitution mutations comprises the H693W substitution. Optionally, the one or more substitution mutations comprises M662W and H693W substitutions.

Thus, in a particular embodiment, the Factor VIII polypeptide comprises a Factor VIII amino acid sequence which comprises one or more substitution mutations, wherein the one or more substitution mutations comprises the M662W substitution. In a further embodiment, the Factor VIII polypeptide comprises a Factor VIII amino acid sequence which comprises one or more substitution mutations, wherein the one or more substitution mutations comprises the H693W substitution. In yet a further embodiment, the Factor VIII polypeptide comprises a Factor VIII amino acid sequence which comprises one or more substitution mutations, wherein the one or more substitution mutations comprise the M662W and H693W substitutions.

Optionally, the Factor VIII polypeptide may comprise a Factor VIII amino acid sequence as set forth in any one of SEQ ID NOs: 28, 30, 35, 36, 40 or 41, or an amino acid sequence which is at least 90% at least 95%, at least 96%, at least 97%, at least 98% or at least 99% identical to an amino acid sequence as set forth in any one of SEQ ID NOs: 28, 30, 35, 36, 40 or 41.

Optionally, the one or more substitution mutations does not comprise substitution of M662 with a less hydrophobic amino acid. Optionally, the one or more substitution mutations does not comprise M6621. Optionally, the one or more substitution mutations does not comprise M662C.

Optionally, the one or more substitution mutations does not comprise M662K. Optionally, the one or more substitution mutations does not comprise M662K if the Factor VIII amino acid sequence comprises an aspartic acid residue at the position corresponding to position 1828 of SEQ ID NO:1 (D1828). Optionally, the one or more substitution mutations does not comprise M662K if the Factor VIII amino acid sequence comprises the sequence MAPTKDEFDCKA at the positions corresponding to positions 1823-1834 of SEQ ID NO:1.

Optionally, the one or more substitution mutations does not comprise substitution of A108. Optionally, the one or more substitution mutations does not comprise substitution of A108 with a more hydrophobic amino acid. Optionally, the one or more substitutions does not comprise A1081.

Optionally, the one or more substitution mutations does not comprise substitution of a negatively-charged amino acid. Optionally, the one or more substitution mutations does not comprise substitution of a negatively-charged amino acid with an uncharged amino acid. Optionally, the one or more substitution mutations does not comprise substitution of D27, E272, E287, D302, D519, E540, E665, D666, E683, D696, D1795, E1829, E1984. Optionally, the one or more substitution mutations does not comprise substitution of D27, E272, E287, D302, D519, E540, E665, D666, E683, D696, D1795, E1829, E1984 with an uncharged amino acid. Optionally, the one or more substitution mutations does not comprise substitution of D27, E272, E287, D302, D519, E540, E665, D666, E683, D696, D1795, E1829, E1984 with alanine or valine. Optionally, the one or more substitution mutations does not comprise D519L, D519Q, D519T or D519V. Optionally, the one or more substitution mutations does not comprise substitution of two or more of D519, E665 and E1984. Optionally, the one or more substitution mutations does not comprise substitution of two or more of D519, E665 and E1984 with alanine or valine.

Optionally, the one or more substitution mutations does not comprise substitution of a positively-charged amino acid. Optionally, the one or more substitution mutations does not comprise substitution of a positively-charged amino acid with an uncharged amino acid. Optionally, the one or more substitution mutations does not comprise substitution of K380, R490, K512, K523, R527, K556, R562, K570 or R571. Optionally, the one or more substitution mutations does not comprise substitution of K380, R490, K512, K523, R527, K556, R562, K570 or R571 with an uncharged amino acid.

Optionally, the one or more substitution mutations does not comprise substitution of 5313, H317, T522, S524, R531, N538, S650, S654, N684, S695, S1791, Q1820, S1949, N1950 or R1966. Optionally, the one or more substitution mutations does not comprise substitution of 5313, H317, T522, S524, R531, N538, S650, S654, N684, S695, S1791, Q1820, S1949, N1950 or R1966 with alanine. Optionally, the one or more substitution mutations does not comprise substitution of Y476, Y664, Y1786 or Y1792. Optionally, the one or more substitution mutations does not comprise substitution of Y476, Y664, Y1786 or Y1792 with phenylalanine.

Optionally, the one or more substitution mutations does not comprise substitution of K659.

Optionally, the one or more substitution mutations does not comprise a non-conservative substitution of K659. Optionally, the one or more substitution mutations does not comprise K659D, K659E, K659Y, K659N, K659Q, K659T, K659S, K659C, K659W, K659F, K659P, K659M, K659V, K659L, K659I, K659Y, K659G or K659A. Optionally, the one or more substitution mutations does not comprise substitution of E665. Optionally, the one or more substitution mutations does not comprise a non-conservative substitution of E665. Optionally, the one or more substitution mutations does not comprise E665V, E665I, E665M, E665N or E665Y.

Optionally, the one or more substitution mutations comprises substitution of K659. Optionally, the one or more substitution mutations comprises K659Q, K659G, K659I or K659F. Optionally, the one or more substitution mutations comprises substitution of E665. Optionally, the one or more substitution mutations comprises E665R, E665Q, E665H, E665K, E665M, E665F, E665W or E665Y.

In certain embodiments, the Factor VIII polypeptide may comprise a Factor VIII amino acid sequence which comprises one or more substitution mutations at an inter-domain interface selected from the A1/A3 domain interface, the A2/A3 domain interface or the A1/C2 domain interface, wherein at least one of the one or more substitution mutations comprises substitution of a pair of amino acids in the respective domains with cysteine residues. Put another way, the at least one or more substitution mutations may comprise substitution of a cysteine residue in a first domain (e.g. the A1 or A2 domain), and substitution of a cysteine residue in a second domain (e.g. the A3 or C2 domain). The pair of amino acids may therefore comprise a first amino (in a first domain) and a second amino acid (in a second domain). Put another way, the at least one or more substitution mutations may comprise substitution of a first amino acid (in a first domain), and a second amino acid (in a second domain) with cysteine residues. Thus, the Factor VIII polypeptide may comprise a Factor VIII amino acid sequence which comprises one or more substitution mutations which comprise substitution of a pair of cognate amino acids in two respective domains of the Factor VIII polypeptide with cysteine residues.

Optionally, the substitution of a pair of amino acids in the respective domains with cysteine residues allows the cysteine residues to form a disulphide bond between the respective domains. The formation of a disulphide bond may be determined by mass spectroscopy, and in particular by the analysing the fragmentation pattern of a polypeptide suspected of containing a disulphide bond, optionally following limited proteolysis, for example as outlined in Gorman et al. 2002. Mass Spectrometry Reviews 21, 183-216. Alternatively, the formation of a disulphide bond may be determined by performing limited proteolysis on a polypeptide and analysing the resulting protein fragments by SDS-PAGE under both reducing and non-reducing conditions, optionally in combination with N-terminal sequencing.

Optionally, the pair of amino acids which are substituted with cysteine residues may be in the A1 and A3 domains. Optionally, the first amino acid may correspond to M147, S149 or S289 of SEQ ID NO:1 and the second amino acid may correspond to E1969, E1970 or N1977 of SEQ ID NO:1. Optionally, the one or more substitution mutations comprises a pair of substitution mutations selected from the list consisting of (i) S289C and N1977C, (ii) M147C and E1970C, and (iii) S149C and E1969C.

Optionally, the pair of amino acids which are substituted with cysteine residues may be in the A2 and A3 domains. Optionally, the first amino acid corresponds to T667, T669, N684, L687, I689, 5695 or F697 of SEQ ID NO: 1 and the second amino acid corresponds to S1791, G1799, A1800, R1803, E1844, S1949, G1981, V1982, or Y1979 of SEQ ID NO: 1. Optionally, the one or more substitution mutations comprises a pair of substitution mutations selected from the list consisting of (i) T669C and V1982C, (ii) L687C and A1800C, (iii) I689C and G1799C, (iv) F697C and S1949C, (v) T667C and G1981C, (vi) T669C and Y1979C, (vii) N684C and S1791C, (viii) L687C and R1803C, and (ix) S695C and E1844C.

Optionally, the pair of amino acids which are substituted with cysteine residues may be in the A1 and C2 domains. Optionally, the first amino acid corresponds to A108, T118 or V137 of SEQ ID NO: 1 and the second amino acid corresponds to N2172, Q2329 or Y2332 of SEQ ID NO: 1. Optionally, the one or more substitution mutations comprises a pair of substitution mutations selected from the list consisting of (i) A108C and Q2329C, (ii) T118C and N2172C, and (iii) V137C and Y2332C.

Optionally, the one or more substitution mutations does not comprise a substitution of any one of the amino acids corresponding to positions 656-667 of SEQ ID NO:1 (YTFKHKMVYEDT) with a cysteine residue. Optionally, the one or more substitution mutations does not comprise a substitution of any one of the amino acids corresponding to positions 1823-1834 of SEQ ID NO:1 (MAPTKDEFDCKA) with a cysteine residue. Optionally, the one or more substitution mutations does not comprise a first substitution mutation comprising a substitution of any one of the amino acids corresponding to positions 656-667 of SEQ ID NO:1 (YTFKHKMVYEDT) with a cysteine residue and a second substitution mutation comprising a substitution of any one of the amino acids corresponding to positions 1823-1834 of SEQ ID NO:1 (MAPTKDEFDCKA) with a cysteine residue.

Optionally, the one or more substitution mutations does not comprise a pair of substitution mutations selected from the list consisting of Y656C and A1834C, T657C and K1833C, K659C and D1831C, H660C and F1830C, K662C and E1829C, M662C and D1828C, V663C and K1827C, Y664C and T1826C, E665C and P1825C, D666C and A1824C, and T667C and M1823C. Optionally, the one or more substitution mutations does not comprise a pair of substitution mutations selected from the list consisting of M662C and D1828C, and Y664C and T1826C. Optionally, the one or more substitution mutations does not comprise R121C and L2302C substitutions. Optionally, the one or more substitution mutations does not comprise a pair of substitution mutations selected from the list consisting of M662C and D1828C, S268C and F673C, I312C and P672C, S313C and A644C, M662C and K1827C, Y664C and T1826C, P264C and Q645C, R282C and T522C, S285C and F673C, H311C and F673C, S314C and A644C, S314C and Q645C, V663C and E1829C, N694C and P1980C, and S695C and E1844C.

In particular embodiments, the one or more substitutions may comprise a pair of substitution mutations selected from the list consisting of (i) L687C and A1800C; (ii) N684C and S1791C; and (iii) S695C and E1844C. Optionally, the Factor VIII polypeptide may comprise a Factor VIII amino acid sequence as set forth in any one of SEQ ID NOs: 37, 38, 39, 42, 43 or 44, or an amino acid sequence as set forth in any one of SEQ ID NOs: 37, 38, 39, 42, 43 or 44

As outlined above, activation of Factor VIII requires proteolytic cleavage by thrombin. Following thrombin cleavage, Factor VIII forms a heterotrimeric complex consisting of the A1 domain, A2 domain, and A3-C1-C2 domains. In the course of activation, Factor VIII undergoes a conformational change. More specifically, in the course of activation, the orientation of the A2 domain relative to the A1 and A3-C1-C2 domains is altered. The A2 domain of activated Factor VIII thus is in a different orientation with respect to the A1, A3, C1 and C2 domains, to inactive Factor VIII. The A2 domain may therefore be seen to pivot with respect to the other domains of Factor VIII during the course of activation. The maximum activity of wild-type Factor VIII is typically seen approximately two minutes following activation.

Without wishing to be bound by theory, it is believed that the conformation change undertaken by the A2 domain may be required for Factor VIII activation. A substitution mutation which stabilises the conformation A2 domain in the orientation of the inactive Factor VIII may therefore inhibit activation of the Factor VIII polypeptide. In certain embodiments, the one or more substitution mutations do not inhibit activation of the Factor VIII polypeptide. Optionally, the one or more substitution mutations do not inhibit activation of the Factor VIII polypeptide relative to the activation of a reference wild-type Factor VIII polypeptide. Optionally, a Factor VIII polypeptide comprising a Factor VIII amino acid sequence which comprises one or more substitution mutations at the A1/A3, A2/A3 or A1/C2 domain interface is not activated more slowly than a reference wild-type Factor VIII polypeptide.

Inhibition of Factor VIII activation may be determined by comparing the time taken to achieve maximum Factor VIII activity following activation with that of a reference wild-type Factor VIII polypeptide. Thrombin may be added to a sample containing a Factor VIII polypeptide and Factor VIII activation may be monitored by taking aliquots of the sample at suitable time points and determining FVIII activity for each aliquot. Optionally FVIII activity may be measured over the course of five, ten, fifteen or twenty minutes following activation. Activity may be determined at a series of time points, e.g. two or more, three or more, four or more, five or more or six or more time points following activation. Optionally Factor VIII activity may be determined immediately prior to activation. By way of representative example, Factor VIII activity may be determined immediately following activation, and further measurements may be taken every thirty seconds following activation. Optionally, relative Factor VIII activity may be determined by calculating the fold-increase in Factor VIII activity at a given time point, compared to Factor VIII activity immediately following activation. Optionally, specific activity may be determined at each time point.

### A Factor VIII Polypeptide Comprising a Factor VIII Amino Acid Sequence Comprising One or More Substitution Mutations

The Factor VIII amino acid sequence may comprise all or part of a signal peptide, such as any of those described herein. Optionally, the signal peptide may be a wild-type Factor VIII signal peptide. Optionally, the signal peptide may comprise SEQ ID NO: 15. Optionally, the signal peptide may be a signal peptide other than a wild-type Factor VIII signal peptide. Optionally, the signal peptide may comprise SEQ ID NO: 18 or 20. The Factor VIII polypeptide may be a mature Factor VIII polypeptide. A “mature Factor VIII polypeptide” is a Factor VIII polypeptide which does not comprise a signal peptide. The signal peptide may have been cleaved following synthesis. The signal peptide may never have been present. For example, the Factor VIII amino acid sequence may never have comprised a signal peptide.

Wild-type Factor VIII polypeptide comprises an A1 domain, an A2 domain, a B domain, an A3 domain, a C1 domain and a C2 domain. The Factor VIII polypeptide of the invention may comprise all or part of each of the A1 domain, the A2 domain, the A3 domain, the B domain, the C1 domain and/or the C2 domain. The sequence of the wild-type Factor VIII polypeptide is set forth in SEQ ID NO: 1.

Optionally, the Factor VIII polypeptide is canine, porcine or human Factor VIII. In a particular embodiment, the Factor VIII polypeptide is human Factor VIII. The amino acid sequence of human Factor VIII is set forth in SEQ ID NO:1. Optionally, the Factor VIII polypeptide may comprise a Factor VIII amino acid sequence that is at least 90%, at least 95%, at least 96%, at least 97%, at least 98%, or at least 99% identical to an amino acid sequence set forth in SEQ ID NO:1. Put another way, the Factor VIII polypeptide may optionally comprise a Factor VIII amino acid sequence that is at least 90%, at least 95%, at least 96%, at least 97%, at least 98%, or at least 99% identical to the corresponding sequence within SEQ ID NO:1. Optionally, the Factor VIII polypeptide may comprise a Factor VIII amino acid that differs from an amino acid sequence set forth in SEQ ID NO:1 only by the one or more substitution mutations.

It is within the capabilities of the person skilled in the art to determine a sequence within SEQ ID NO: 1 which corresponds to the Factor VIII amino acid sequence of a Factor VIII polypeptide. For example, the person skilled in the art merely needs to perform a sequence alignment of the Factor VIII amino acid sequence with SEQ ID NO: 1 using a suitable alignment algorithm such as that of Needleman and Wunsch described above, and determine which region of SEQ ID NO: 1 corresponds to the Factor VIII amino acid (i.e. which sequence of SEQ ID NO: 1 aligns to the Factor VIII amino acid sequence).

Optionally, the Factor VIII polypeptide may comprise a Factor VIII amino acid sequence which comprises an amino acid sequence that is at least 90%, at least 95%, at least 96%, at least 97%, at least 98%, or at least 99% identical to an amino acid sequence comprising at least 1349, at least 1357, at least 1368, at least 1376, at least 1377, at least 1385, at least 1386, at least 1391, at least 1394, at least 1418 or at least 1421 amino acids of SEQ ID NO: 1. The Factor VIII polypeptide may be at least 90% identical to at least 1349, at least 1357, at least 1368, at least 1376, at least 1377, at least 1385, at least 1386, at least 1391, at least 1394, at least 1418 or at least 1421 contiguous amino acids of SEQ ID NO: 1. Alternatively, the Factor VIII polypeptide may be at least 90% identical to at least 1349, at least 1357, at least 1368, at least 1376, at least 1377, at least 1385, at least 1386, at least 1391, at least 1394, at least 1418 or at least 1421 amino acids from up to 5, up to 4, up to 3, or up to 2 regions of SEQ ID NO: 1. Optionally, the Factor VIII polypeptide may comprise a Factor VIII amino acid sequence that differs from an amino acid sequence comprising at least 1349 amino acids of SEQ ID NO:1 by 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 substitution mutations. Optionally, the Factor VIII polypeptide may comprise a Factor VIII amino acid that differs from an amino acid sequence comprising at least 1349 amino acids of SEQ ID NO:1 only by the one or more substitution mutations.

Optionally, the Factor VIII polypeptide may comprise a Factor VIII amino acid sequence which comprises an amino acid sequence that is at least 90%, at least 95%, at least 96%, at least 97%, at least 98%, or at least 99% identical to the amino acid sequence set forth in SEQ ID NO: 1. Optionally, the Factor VIII polypeptide may comprise a Factor VIII amino acid sequence that differs from the amino acid sequence set forth in SEQ ID NO:1 by 20 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 substitution mutations. Optionally, the Factor VIII polypeptide may comprise a Factor VIII amino acid that differs from the amino acid sequence set forth in SEQ ID NO:1 only by the one or more substitution mutations.

The Factor VIII polypeptide may comprise part of the B domain, or the Factor VIII polypeptide may not comprise the B domain. According to a particular embodiment, the Factor VIII polypeptide does not comprise a B domain. The Factor VIII polypeptide may therefore be a beta domain deleted (BDD) Factor VIII polypeptide.

Optionally, the Factor VIII polypeptide may comprise a Factor VIII amino acid sequence which comprises an amino acid sequence that is at least 90%, at least 95%, at least 96%, at least 97%, at least 98%, or at least 99% identical to an amino acid sequence comprising at least 1349, at least 1357, at least 1368, at least 1376, at least 1377, at least 1385, at least 1386, at least 1391, at least 1394, at least 1418 or at least 1421 amino acids of SEQ ID NOs: 3 or 5. The Factor VIII polypeptide may be at least 90% identical to at least 1349, at least 1357, at least 1368, at least 1376, at least 1377, at least 1385, at least 1386, at least 1391, at least 1394, at least 1418 or at least 1421 contiguous amino acids of SEQ ID NOs: 3 or 5. Alternatively, the Factor VIII polypeptide may be at least 90% identical to at least 1349, at least 1357, at least 1368, at least 1376, at least 1377, at least 1385, at least 1386, at least 1391, at least 1394, at least 1418 or at least 1421 amino acids from up to 5, up to 4, up to 3, or up to 2 regions of SEQ ID NOs: 3 or 5. Optionally, the Factor VIII polypeptide may comprise a Factor VIII amino acid sequence that differs from an amino acid sequence comprising at least 1349, at least 1357, at least 1368, at least 1376, at least 1377, at least 1385, at least 1386, at least 1391, at least 1394, at least 1418 or at least 1421 amino acids of SEQ ID NOs: 3 or 5 by 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 substitution mutations. Optionally, the Factor VIII polypeptide may comprise a Factor VIII amino acid that differs from an amino acid sequence comprising at least 1349, at least 1357, at least 1368, at least 1376, at least 1377, at least 1385, at least 1386, at least 1391, at least 1394, at least 1418 or at least 1421 amino acids of SEQ ID NOs: 3 or 5 only by the one or more substitution mutations.

Optionally, the Factor VIII polypeptide may comprise a Factor VIII amino acid sequence which comprises an amino acid sequence that is at least 90%, at least 95%, at least 96%, at least 97%, at least 98%, or at least 99% identical to the amino acid sequence set forth in SEQ ID NOs: 3 or 5. Optionally, the Factor VIII polypeptide may comprise a Factor VIII amino acid sequence that differs from the amino acid sequence set forth in SEQ ID NOs: 3 or 5 by 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 substitution mutations. Optionally, the Factor VIII polypeptide may comprise a Factor VIII amino acid that differs from the amino acid sequence set forth in SEQ ID NOs: 3 or 5 only by the one or more substitution mutations.

Any or all of the domains of the Factor VIII polypeptide may comprise one or more modifications, such as substitutions, insertions and/or deletions in addition to the one or more substitution mutations defined herein, when the Factor VIII amino acid sequence of the domain is compared to a wild-type Factor VIII amino acid sequence.

The Factor VIII polypeptide may comprise an amino acid sequence which is at least 90% identical to at least 300 amino acids of an A1 domain. The Factor VIII polypeptide may comprise an amino acid sequence which is at least 95%, at least 98%, at least 99% or 100% identical to at least 300 amino acids (optionally at least 300 contiguous amino acids) of an A1 domain. The Factor VIII polypeptide may comprise an amino acid sequence which is at least 90% identical to an A1 domain. The Factor VIII polypeptide may comprise an amino acid sequence which is at least 95%, at least 98%, at least 99% or 100% identical to an A1 domain. The Factor VIII polypeptide may comprise an amino acid sequence which is at least 90% identical to at least 300 amino acids of the amino acids corresponding to positions 1 to 329 of SEQ ID NO: 1. The Factor VIII polypeptide may comprise an amino acid sequence which is at least 95%, at least 98%, at least 99% or 100% identical to at least 300 amino acids of the amino acids corresponding to positions 1 to 329 of SEQ ID NO: 1. The Factor VIII polypeptide may comprise an amino acid sequence which is at least 90% identical to the amino acids corresponding to positions 1 to 329 of SEQ ID NO: 1. The Factor VIII polypeptide may comprise an amino acid sequence which is at least 95%, at least 98%, at least 99% or 100% identical to the amino acids corresponding to positions 1 to 329 of SEQ ID NO: 1.

The Factor VIII polypeptide may comprise an amino acid sequence which is at least 90% identical to at least 300 amino acids of an A2 domain. The Factor VIII polypeptide may comprise an amino acid sequence which is at least 95%, at least 96%, at least 98%, at least 99% or 100% identical to at least 300 amino acids of an A2 domain (optionally at least 300 contiguous amino acids). The Factor VIII polypeptide may comprise an amino acid sequence which is at least 90% identical to an A2 domain. The Factor VIII polypeptide may comprise an amino acid sequence which is at least 95%, at least 96%, at least 98%, at least 99% or 100% identical to an A2 domain. The Factor VIII polypeptide may comprise an amino acid sequence which is at least 90% identical to at least 300 amino acids of the amino acids corresponding to positions 380 to 711 of SEQ ID NO:1. The Factor VIII polypeptide may comprise an amino acid sequence which is at least 95%, at least 98%, at least 99% or 100% identical to at least 300 amino acids of the amino acids corresponding to positions 380 to 711 of SEQ ID NO:1. The Factor VIII polypeptide may comprise an amino acid sequence which is at least 90% identical to the amino acids corresponding to positions 380 to 711 of SEQ ID NO:1. The Factor VIII polypeptide may comprise an amino acid sequence which is at least 95%, at least 98%, at least 99% or 100% identical to the amino acids corresponding to positions 380 to 711 of SEQ ID NO:1.

The Factor VIII polypeptide may comprise an amino acid sequence which is at least 90% identical to at least 300 amino acids of an A3 domain. The Factor VIII polypeptide may comprise an amino acid sequence which is at least 95%, at least 96%, at least 98%, at least 99% or 100% identical to at least 300 amino acids of an A3 domain (optionally at least 300 contiguous amino acids). The Factor VIII polypeptide may comprise an amino acid sequence which is at least 90% identical to an A3 domain. The Factor VIII polypeptide may comprise an amino acid sequence which is at least 95%, at least 96%, at least 98%, at least 99% or 100% identical to an A3 domain. The Factor VIII polypeptide may comprise an amino acid sequence which is at least 90% identical to at least 300 amino acids of the amino acids corresponding to positions 1694 to 2021 of SEQ ID NO:1. The Factor VIII polypeptide may comprise an amino acid sequence which is at least 95%, at least 98%, at least 99% or 100% identical to at least 300 amino acids of the amino acids corresponding to positions 1694 to 2021 of SEQ ID NO:1. The Factor VIII polypeptide may comprise an amino acid sequence which is at least 90% identical to the amino acids corresponding to positions 1694 to 2021 of SEQ ID NO:1. The Factor VIII polypeptide may comprise an amino acid sequence which is at least 95%, at least 98%, at least 99% or 100% identical to the amino acids corresponding to positions 1694 to 2021 of SEQ ID NO:1.

The Factor VIII polypeptide may comprise an amino acid sequence which is at least 90% identical to at least 130, at least 135 or at least 140 amino acids of a C1 domain. The Factor VIII polypeptide may comprise an amino acid sequence which is at least 95%, at least 96%, at least 98%, at least 99% or 100% identical to at least 130, at least 135 or at least 140 amino acids of a C1 domain (optionally at least 130 contiguous amino acids). The Factor VIII polypeptide may comprise an amino acid sequence which is at least 90% identical to a C1 domain. The Factor VIII polypeptide may comprise an amino acid sequence which is at least 95%, at least 96%, at least 98%, at least 99% or 100% identical to a C1 domain. The Factor VIII polypeptide may comprise an amino acid sequence which is at least 90% identical to at least 130, at least 135 or at least 140 amino acids of the amino acids corresponding to positions 2021 to 2169 of SEQ ID NO:1. The Factor VIII polypeptide may comprise an amino acid sequence which is at least 95%, at least 98%, at least 99% or 100% identical to at least 130, at least 135 or at least 140 amino acids of the amino acids corresponding to positions 2021 to 2169 of SEQ ID NO:1. The Factor VIII polypeptide may comprise an amino acid sequence which is at least 90% identical to the amino acids corresponding to positions 2021 to 2169 of SEQ ID NO:1. The Factor VIII polypeptide may comprise an amino acid sequence which is at least 95%, at least 98%, at least 99% or 100% identical to the amino acids corresponding to positions 2021 to 2169 of SEQ ID NO:1.

The Factor VIII polypeptide may comprise an amino acid sequence which is at least 90% identical to at least 130, at least 135 or at least 140 amino acids of a C2 domain (optionally at least 130 contiguous amino acids). The Factor VIII polypeptide may comprise an amino acid sequence which is at least 95%, at least 96%, at least 98%, at least 99% or 100% identical to at least 130, at least 135 or at least 140 amino acids of a C2 domain. The Factor VIII polypeptide may comprise an amino acid sequence which is at least 90% identical to a C2 domain. The Factor VIII polypeptide may comprise an amino acid sequence which is at least 95%, at least 96%, at least 98%, at least 99% or 100% identical to a C2 domain. The Factor VIII polypeptide may comprise an amino acid sequence which is at least 90% identical to at least 130, at least 135 or at least 140 amino acids of the amino acids corresponding to positions 2174 to 2326 of SEQ ID NO:1. The Factor VIII polypeptide may comprise an amino acid sequence which is at least 95%, at least 98%, at least 99% or 100% identical to at least 130, at least 135 or at least 140 amino acids of the amino acids corresponding to positions 2174 to 2326 of SEQ ID NO:1. The Factor VIII polypeptide may comprise an amino acid sequence which is at least 90% identical to the amino acids corresponding to positions 2174 to 2326 of SEQ ID NO:1. The Factor VIII polypeptide may comprise an amino acid sequence which is at least 95%, at least 98%, at least 99% or 100% identical to the amino acids corresponding to positions 2174 to 2326 of SEQ ID NO:1.

Optionally, the Factor VIII polypeptide may comprise a full or partial B domain. The Factor VIII polypeptide may comprise an amino acid sequence which is at least 90% identical to at least 820 amino acids of a B domain. The Factor VIII polypeptide may comprise an amino acid sequence which is at least 95%, at least 96%, at least 98%, at least 99% or 100% identical to at least 820 amino acids of a B domain (optionally at least 820 contiguous amino acids). The Factor VIII polypeptide may comprise an amino acid sequence which is at least 90% identical to a B domain. The Factor VIII polypeptide may comprise an amino acid sequence which is at least 95%, at least 96%, at least 98%, at least 99% or 100% identical to a B domain. The Factor VIII polypeptide may comprise an amino acid sequence which is at least 90% identical to at least 800 amino acids of the amino acids corresponding to positions 741 to 1648 of SEQ ID NO:1. The Factor VIII polypeptide may comprise an amino acid sequence which is at least 95%, at least 98%, at least 99% or 100% identical to at least 800 amino acids of the amino acids corresponding to positions 741 to 1648 of SEQ ID NO:1. The Factor VIII polypeptide may comprise an amino acid sequence which is at least 90% identical to the amino acids corresponding to positions 741 to 1648 of SEQ ID NO:1. The Factor VIII polypeptide may comprise an amino acid sequence which is at least 95%, at least 98%, at least 99% or 100% identical to the amino acids corresponding to positions 741 to 1648 of SEQ ID NO:1.

Optionally, the Factor VIII polypeptide may comprise a modified beta domain related (BDR) region. The wild-type BDR region corresponds to the region between positions 713 and 1697 of SEQ ID NO:1. The Factor VIII polypeptide may comprise a modified BDR region which comprises a maximum of 88 amino acids.

The wild-type BDR region corresponds to the region between positions 713 and 1697 of SEQ ID NO: 1. When referring to amino acid positions, the term “between” does not include the specified positions. Thus the wild-type BDR region starts at the position that corresponds to position 714 of SEQ ID NO: 1 and ends at the position that corresponds to position 1696 of SEQ ID NO: 1. The wild-type BDR region comprises the beta domain of Factor VIII. The wild-type BDR region may be the region between positions 713 and 1697 of SEQ ID NO: 1. The wild-type BDR region may start at the position 714 of SEQ ID NO: 1 and may end at position 1696 of SEQ ID NO: 1. The wild-type BDR region may be the region from another wild-type Factor VIII amino acid sequence which corresponds to the region between positions 713 and 1697 of SEQ ID NO: 1. The wild-type BDR region may be the region from another wild-type Factor VIII amino acid sequence which corresponds to the region that starts at position 714 of SEQ ID NO: 1 and ends at position 1696 of SEQ ID NO: 1.

It is within the capabilities of the person skilled in the art to determine the region of a wild-type Factor VIII amino acid sequence which “corresponds to” the region between positions 713 and 1697 of SEQ ID NO: 1. For example, the person skilled in the art merely needs to perform a sequence alignment of the wild-type Factor VIII amino acid sequence with SEQ ID NO: 1 using a suitable alignment algorithm such as that of Needleman and Wunsch described above, and determine which region of the wild-type Factor VIII amino acid sequence aligns with the region between positions 713 and 1697 of

SEQ ID NO: 1. For example, the person skilled in the art may determine which region of the wild-type Factor VIII amino acid sequence aligns with positions 714 to 1696 of SEQ ID NO: 1.

The “modified BDR region” is not identical to the wild-type BDR region. The modified BDR region is shorter than wild-type BDR region. A skilled person can readily determine whether the modified BDR region of a Factor VIII polypeptide “is modified relative to wild-type BDR region”. For example, the person skilled in the art merely needs to perform a sequence alignment of the Factor VIII amino acid sequence with a wild-type Factor VIII amino acid sequence of the Factor VIII polypeptide using a suitable alignment algorithm such as that of Needleman and Wunsch described above, determine the region of the Factor VIII amino acid sequence (modified BDR region) which aligns with the region of wild-type Factor VIII amino acid sequence (wild-type BDR region) that corresponds to the region between positions 713 and 1697 of SEQ ID NO: 1, and compare the amino acid sequence between the region in the Factor VIII amino acid sequence (modified BDR region) and the region in the wild-type Factor VIII amino acid sequence (wild-type BDR region). If there are any differences, then the region is a modified BDR region.

Since the modified BDR region of the Factor VIII polypeptide of the invention comprises a maximum of 88 amino acids, there will be one or more gap(s) in the Factor VIII amino acid sequence in the modified BDR region compared to wild-type BDR region when the Factor VIII amino acid sequence is aligned with the wild-type Factor VIII amino acid sequence. Thus, the modified BDR region of the Factor VIII polypeptide of the invention comprises one or more deletion(s) when the modified BDR region is compared to a wild-type BDR region. The modified BDR region may also have one or more modifications, such as substitutions and/or insertions, when the modified BDR region is compared to a wild-type BDR region. The modified BDR region may comprise one or more amino acids found in a wild-type BDR region. For example, the modified BDR region may comprise one or more stretches of amino acids found in a wild-type BDR region.

The modified BDR region “comprises a maximum” of a specified number of amino acids. This means that the modified BDR region comprises no more than the specified number of amino acids. The Factor VIII amino acid sequence may comprise further amino acids, but not in the modified BDR region. For example, if the modified BDR region “comprises a maximum of 88 amino acids”, the modified BDR region comprises no more than 88 amino acids. As a further example, if the modified BDR region “comprises a maximum of 74 amino acids”, the modified BDR region comprises no more than 74 amino acids.

The “Factor VIII polypeptide” is functional. A functional Factor VIII polypeptide is one which can, when activated by thrombin, form an enzymatic complex with Factor IXa, phospholipids and calcium, and the enzymatic complex can catalyse the conversion of Factor X to Factor Xa.

It is within the abilities of the person skilled in the art to determine whether a Factor VIII polypeptide is functional. The person skilled in the art merely needs to determine the specific activity of the Factor VIII polypeptide. If the specific activity of the polypeptide is at least 20% of the specific activity of a wild-type Factor VIII polypeptide such as a Factor VIII polypeptide of SEQ ID NO: 1, then it is functional. The activity of the Factor VIII polypeptide can be analysed using a chromogenic assay, such as a chromogenic assay that measures cofactor activity. For example, a suitable chromogenic assay is as follows. The Factor VIII polypeptide (is mixed with human Factor X polypeptide and Factor IXa polypeptide, thrombin, phospholipids and calcium). The thrombin activates the Factor VIII polypeptide to form Factor VIIIa polypeptide. The thrombin-activated Factor VIII polypeptide forms an enzymatic complex with Factor IXa polypeptide, phospholipids and calcium, which enzymatic complex can catalyse the conversion of Factor X polypeptide to Factor Xa polypeptide. The activity of the Factor Xa polypeptide can catalyse cleavage of a chromogenic substrate (e.g. SXa-11) to produce pNA. The level of pNA generated can be measured by determining colour development at 405 nm (e.g. measured by absorbance). Factor X polypeptide, and therefore Factor Xa polypeptide, is provided in excess.

Therefore the limiting factor is Factor VIIIa polypeptide. Thus, the level of pNA generated is proportional to the amount of the Factor Xa polypeptide generated by Factor FVIIIa polypeptide in the sample, which is proportional to the activity of Factor FVIIIa polypeptide in the sample. The activity of Factor FVIIIa polypeptide in the sample is a measure of the cofactor activity of the Factor FVIII polypeptide in the sample.

For example, a suitable chromogenic assay is the BIOPHEN FVIII:C assay (Ref: 221406) manufactured by HYPHEN BioMed as used in the Examples. The activity of the Factor VIII polypeptide may be measured using the BIOPHEN FVIII:C assay.

The activity of the Factor VIII polypeptide can be analysed using a clotting assay. The assay may be a one-stage clotting assay. For example, a suitable clotting assay (a one-stage clotting assay) is as follows.

Since Factor VIII is part of the clotting cascade, a Factor VIII polypeptide that has increased activity will catalyse blood clotting more quickly than a Factor VIII polypeptide that has a lower activity. The Factor VIII polypeptide is mixed with platelet poor plasma, and incubated at 37° C. Then phospholipid and a contact activation pathway activator such as Kaolin or SynthaSIL APTT reagent are added. Calcium is then added, and the user measures the time taken for clotting to occur. Clot formation can be assessed directly by a magnetic steel ball method. Clot formation may be measured using a rotating cuvette assay in which a steel ball remains stationary in a magnetic field until the formation of fibrin strands around the ball produces movement and a change in the magnetic field can be detected. Alternatively, clot formation may be measured using a rotating steel ball assay in which a steel ball is rotated under the influence of a magnet until the formation of fibrin strands around the ball stops it rotating, which can be detected by a sensor. In either event, coagulation time may be recorded.

In patients, such as human patients, the activity of the Factor VIII polypeptide may be measured by taking a blood sample from the patient, or by performing an assay on a blood sample that has been taken from the patient.

The activity of the Factor VIII polypeptide can be analysed using a tail clip assay. A suitable tail clip assay may involve administering Factor VIII polypeptide, or polynucleotide comprising a Factor VIII nucleotide sequence in the context of a gene therapy, to mice such as knock-out mice deficient in Factor VIII. The tails of the mice are then clipped, and the time taken for the cut in the tails to clot is measured. The duration of bleeding provides a relative measure of the activity of the administered Factor VIII, for example a Factor VIII polypeptide comprising a Factor VIII amino acid sequence comprising a modified BDR region vs wild-type Factor VIII, or a Factor VIII polypeptide which is encoded by a Factor VIII nucleotide sequence wherein at least a portion of the Factor VIII nucleotide sequence is not wild-type vs wild-type Factor VIII.

The modified BDR region comprises a maximum of 88 amino acids. The modified BDR region may comprise a maximum of 87, 85, 80, 75, 70, 65, 60, 55, 50, or 45 amino acids. Preferably, the modified BDR region may comprise a maximum of 74 amino acids. The modified BDR region may comprise a maximum of 54 amino acids. The modified BDR region may comprise a maximum of 47 amino acids. The modified BDR region may comprise a maximum of 45 amino acids.

The modified BDR region may comprise at least 20, at least 25, at least 28, at least 30, at least 35, at least 40, at least 45, at least 50, at least 54, at least 55, at least 57, at least 58, at least 60, or at least 65 amino acids. The modified BDR region may comprise at least 28 amino acids. The modified BDR region may comprise at least 30 amino acids. The modified BDR region may comprise at least 54 amino acids. The modified BDR region may comprise at least 57 amino acids. The modified BDR region may comprise at least 58 amino acids.

Optionally, the Factor VIII polypeptide may comprise a Factor VIII amino acid sequence which comprises amino acids corresponding to positions 1 to 722 and 1670 to 2332 of SEQ ID NO: 1. Optionally, the Factor VIII polypeptide may comprise a Factor VIII amino acid sequence which consists of amino acids corresponding to positions 1 to 722 and 1670 to 2332 of SEQ ID NO: 1. Optionally, the Factor VIII polypeptide may comprise a Factor VIII amino acid sequence which comprises amino acids corresponding to positions 1 to 731 and 1670 to 2332 of SEQ ID NO: 1. Optionally, the Factor VIII polypeptide may comprise a Factor VIII amino acid sequence which consists of amino acids corresponding to positions 1 to 731 and 1670 to 2332 of SEQ ID NO: 1. Optionally, the Factor VIII polypeptide may comprise a Factor VIII amino acid sequence which comprises amino acids corresponding to positions 1 to 743 and 1638 to 2332 of SEQ ID NO:1 (an ‘SQ’ Factor VIII polypeptide). Optionally, the Factor VIII polypeptide may consist of amino acids corresponding to positions 1 to 743 and 1638 to 2332 of SEQ ID NO:1. Optionally, the Factor VIII amino acid sequence may comprise the SQ linker sequence SFSQNPPVLKRHQR.

Optionally, the Factor VIII polypeptide may comprise a Factor VIII amino acid sequence which comprises an amino acid sequence that is at least 90%, at least 95%, at least 96%, at least 97%, at least 98%, at least 99% or 100% identical to the amino acid sequence set forth in SEQ ID NO: 28 or 30. Optionally, the Factor VIII polypeptide may comprise a Factor VIII amino acid sequence which consists of an amino acid sequence that is at least 90%, at least 95%, at least 96%, at least 97%, at least 98%, at least 99% or 100% identical to the amino acid sequence set forth in SEQ ID NO: 28 or 30. Optionally, the Factor VIII polypeptide may comprise a Factor VIII amino acid sequence which comprises the amino acid sequence set forth in SEQ ID NO: 28 or 30. Optionally, the Factor VIII polypeptide may comprise a Factor VIII amino acid sequence which consists of the amino acid sequence set forth in SEQ ID NO: 28 or 30.

### A Polynucleotide Comprising a Factor VIII Nucleotide Sequence

The invention provides a polynucleotide comprising a Factor VIII nucleotide sequence, wherein the Factor VIII nucleotide sequence encodes a Factor VIII polypeptide of the invention.

The term “polynucleotide” refers to a polymeric chain of nucleotides of any length, e.g. deoxyribonucleotides, ribonucleotides, or analogues thereof. For example, the polynucleotide may comprise DNA (deoxyribonucleotides) or RNA (ribonucleotides). The polynucleotide may consist of DNA. The polynucleotide may be mRNA. Since the polynucleotide may comprise RNA or DNA, all references to T (thymine) nucleotides may be replaced with U (uracil).

The Factor VIII nucleotide sequence encodes a Factor VIII polypeptide. A “sequence” that “encodes” refers to a nucleotide sequence comprising codons that encode the encoded amino acid sequence. For example, a nucleotide sequence that encodes a Factor VIII polypeptide comprises codons that encode the Factor VIII polypeptide. A nucleotide sequence that encodes wild-type Factor VIII is provided in SEQ ID NO:2. The Factor VIII nucleotide sequence may comprise one or more regions of one or more non-coding nucleotides, such as introns. The regions of non-coding nucleotides may interrupt the sequence of codons that encode the encoded amino acid sequence. Thus the sequence of codons that encode the encoded amino acid sequence may be contiguous in sequence or separated by one or more regions of non-coding nucleotides. Preferably the Factor VIII nucleotide sequence does not comprise any non-coding nucleotides. Herein, the stop codon will not be considered non-coding nucleotides.

The following Table describes codons that encode each amino acid:

TABLE 3

| Amino Acid | Codon |
|---|---|
| Phenylalanine | TTC |
| | TTT |
| Leucine | TTA |
| | TTG |
| | CTT |
| | CTC |
| | CTA |
| | CTG |
| Isoleucine | ATT |
| | ATC |
| | ATA |
| Methionine | ATG |
| Valine | GTT |
| | GTC |
| | GTA |
| | GTG |
| Serine | TCT |
| | TCC |
| | TCA |
| | TCG |
| | AGT |
| | AGC |
| Arginine | CGT |
| | CGC |
| | CGA |
| | CGG |
| | AGA |
| | AGG |
| Proline | CCT |
| | CCC |
| | CCA |
| | CCG |
| Threonine | ACT |
| | ACC |
| | ACA |
| | ACG |
| Alanine | GCT |
| | GCC |
| | GCA |
| | GCG |
| Tyrosine | TAT |
| | TAC |
| Histidine | CAT |
| | CAC |
| Glutamine | CAA |
| | CAG |
| Glycine | GGT |
| | GGC |
| | GGA |
| | GGG |
| Asparagine | AAT |
| | AAC |
| Lysine | AAA |
| | AAG |
| Aspartic | GAT |
| Acid | GAC |
| Glutamic | GAA |
| Acid | GAG |
| Cysteine | TGT |
| | TGC |
| Tryptophan | TGG |

The corresponding RNA codons will contain Us in place of the Ts in the Table above.

The Factor VIII nucleotide sequence may encode a mature Factor VIII polypeptide. Optionally, the Factor VIII nucleotide sequence does not encode all or a portion of a signal peptide. The Factor VIII nucleotide sequence may encode all or part of a signal peptide, such as any or part of any of the signal peptides described herein.

The Factor VIII nucleotide sequence of the invention may be codon-optimised. As mentioned above, the genetic code is degenerate, and many amino acids may be encoded by more than one alternative codon. However, the genetic code of different organisms, tissues or cells may be biased towards using one particular codon for encoding a particular amino acid. A nucleotide sequence that is “codon-optimised” may be optimised for expression in a particular host cell or organism, for example expression in human liver cells. Preferably, a codon-optimised nucleic acid sequence is modified relative to a Factor VIII nucleotide sequence whilst the amino acid sequence encoded by the nucleotide sequence is not modified.

A polypeptide encoded by a codon optimised Factor VIII nucleotide sequence may be expressed in human liver cells at higher levels compared to a reference wild-type Factor VIII nucleotide sequence.

Thus the Factor VIII polypeptide encoded by the Factor VIII nucleotide sequence may be expressed at a higher level than a polypeptide encoded by a wild-type Factor VIII nucleotide sequence when the sequences are expressed in human liver cells. For example, a polypeptide encoded by a codon-optimised Factor VIII nucleotide sequence may be expressed in human liver cells at least 1.1 fold, at least 1.2 fold, at least 1.5 fold, at least 1.8 fold, at least 2 fold, at least 5 fold, at least 10 fold, at least 20 fold, at least 30 fold, at least 40 fold, or at least 50 fold higher compared to a reference wild-type Factor VIII nucleotide sequence. The “reference wild-type Factor VIII nucleotide sequence” may be any Factor VIII nucleotide sequence that uses wild-type codons to encode a Factor VIII polypeptide (which may itself be a wild-type Factor VIII polypeptide or may be a modified Factor VIII polypeptide such as a Factor VIII polypeptide of the invention comprising a modified BDR), such as the Factor VIII nucleotide sequence of SEQ ID NO: 2. Optionally, the reference wild-type Factor VIII nucleotide is an “equivalent” Factor VIII nucleotide sequence i.e. the reference wild-type Factor VIII nucleotide encodes the same Factor VIII polypeptide as the Factor VIII polynucleotide to which it is being compared.

By describing a nucleotide sequence as “codon-optimised”, all the codons do not need to be optimised. Thus the portion of the Factor VIII nucleotide sequence that codon-optimised may comprise one or more alternative codon(s) in place of the wild-type codon(s) when compared to the corresponding portion of a wild-type Factor VIII nucleotide sequence. If the Factor VIII nucleotide sequence comprises a portion that is codon-optimised, the polypeptide encoded by the Factor VIII nucleotide sequence may be expressed at a higher level than a polypeptide encoded by a wild-type Factor VIII nucleotide sequence. The portion of the Factor VIII nucleotide sequence that is codon-optimised may be codon-optimised for expression in human liver cells. Thus the polypeptide encoded by the Factor VIII nucleotide sequence may be expressed at a higher level than the polypeptide encoded by an equivalent non-codon-optimised (e.g. a wild-type) Factor VIII nucleotide sequence when the sequences are expressed in human liver cells. An “equivalent” non-codon optimised Factor VIII nucleotide sequence is identical (i.e. encodes the same Factor VIII polypeptide and comprises the same transcriptional regulatory elements etc.) except the codons used to encode the Factor VIII polypeptide will correspond to the corresponding codons of a wild-type Factor VIII sequence such as SEQ ID NO: 2. Typically, the codon-optimised portion of the Factor VIII nucleotide sequence does not comprise the stop codon.

The Factor VIII nucleotide sequence of the invention may comprise one or more alternative codon(s) in place of the wild-type codon(s), wherein an “alternative” codon is a codon which has a different sequence to the wild-type codon but which encodes the same amino acid as the wild-type codon (i.e. a degenerate codon). Table 3 describes the codons which encode each amino acid.

A codon-optimised nucleotide sequence may comprise at least one more “preferred” codons than a corresponding nucleotide sequence which is not codon-optimised. A codon-optimised nucleotide sequence may comprise a higher percentage of “preferred” codons than a corresponding nucleotide sequence which is not codon-optimised. A codon-optimised nucleotide sequence may comprise at least one fewer “non-preferred” codons than a corresponding nucleotide sequence which is not codon-optimised. A codon-optimised nucleotide sequence may comprise a lower percentage of “non-preferred” codons than a corresponding nucleotide sequence which is not codon-optimised. Preferred codons for expression of Factor VIII in human liver cells are underlined in Table 3.

Optionally, the Factor VIII nucleotide sequence encoding the Factor VIII amino acid sequence may be at least 85%, at least 90%, at least 95%, at least 96%, at least 97%, at least 98%, or at least 99% identical to a nucleotide sequence comprising at least 4047, at least 4071, at least 4104, at least 4128, at least 4131, at least 4155, at least 4158, at least 4173, at least 4182, at least 4254 or at least 4263 nucleotides (optionally at least 4047, at least 4071, at least 4104, at least 4128, at least 4131, at least 4155, at least 4158, at least 4173, at least 4182, at least 4254 or at least 4263 contiguous nucleotides, or at least 4047, at least 4071, at least 4104, at least 4128, at least 4131, at least 4155, at least 4158, at least 4173, at least 4182, at least 4254 or at least 4263 nucleotides from up to 5, up to 4, up to 3 or up to 2 regions of SEQ ID NO:4) of SEQ ID NO:4. Optionally, the Factor VIII nucleotide sequence may comprise a nucleotide sequence that differs from the nucleotide sequence comprising at least 4047, at least 4071, at least 4104, at least 4128, at least 4131, at least 4155, at least 4158, at least 4173, at least 4182, at least 4254 or at least 4263 nucleotides of SEQ ID NO:4 by 40 or fewer, 35 or fewer, 30 or fewer, 25 or fewer, 20 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 nucleotides. Optionally, the Factor VIII nucleotide sequence may comprise a nucleotide sequence that differs from the nucleotide sequence comprising at least 4047, at least 4071, at least 4104, at least 4128, at least 4131, at least 4155, at least 4158, at least 4173, at least 4182, at least 4254 or at least 4263 nucleotides of SEQ ID NO:4 only by alternative codons encoding the one or more substitution mutations.

Optionally, the Factor VIII nucleotide sequence encoding the Factor VIII amino acid sequence may be at least 85%, at least 90%, at least 95%, at least 96%, at least 97%, at least 98%, or at least 99% identical to the nucleotide sequence set forth in SEQ ID NO:6. Optionally, the Factor VIII nucleotide sequence may comprise a nucleotide sequence that differs from the nucleotide set forth in SEQ ID NO:6 by 43 or fewer, 40 or fewer, 35 or fewer, 30 or fewer, 25 or fewer, 20 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 nucleotides. Optionally, the Factor VIII nucleotide sequence may comprise a nucleotide sequence that differs from the nucleotide sequence set forth in SEQ ID NO:6 only by alternative codons encoding the one or more substitution mutations.

Optionally, the Factor VIII nucleotide sequence encoding the Factor VIII amino acid sequence may comprise a nucleotide sequence that is at least 85%, at least 90%, at least 95%, at least 96%, at least 97%, at least 98%, at least 99% or 100% identical to the nucleotide set forth in SEQ ID NO:29. Optionally, the Factor VIII nucleotide sequence may consist of a nucleotide sequence that is at least 85%, at least 90%, at least 95%, at least 96%, at least 97%, at least 98%, at least 99% or 100% identical to the nucleotide sequence set forth in SEQ ID NO:29. Optionally, the Factor VIII nucleotide sequence may comprise the nucleotide sequence set forth in SEQ ID NO:29. Optionally, the Factor VIII nucleotide sequence may consist of the nucleotide sequence set forth in SEQ ID NO:29.

Optionally, the Factor VIII nucleotide sequence encoding the Factor VIII amino acid sequence may comprise a nucleotide sequence that is at least 85%, at least 90%, at least 95%, at least 96%, at least 97%, at least 98%, at least 99% or 100% identical to the nucleotide set forth in SEQ ID NO:31. Optionally, the Factor VIII nucleotide sequence encoding the Factor VIII amino acid sequence may comprise a nucleotide sequence that is at least 85%, at least 90%, at least 95%, at least 96%, at least 97%, at least 98%, at least 99% or 100% identical to a nucleotide sequence comprising at least 4047 nucleotides of SEQ ID NO: 31. Optionally, the Factor VIII nucleotide sequence may consist of a nucleotide sequence that is at least 85%, at least 90%, at least 95%, at least 96%, at least 97%, at least 98%, at least 99% or 100% identical to the nucleotide sequence set forth in SEQ ID NO:31. Optionally, the Factor VIII nucleotide sequence may comprise the nucleotide sequence set forth in SEQ ID NO:31. Optionally, the Factor VIII nucleotide sequence may consist of the nucleotide sequence set forth in SEQ ID NO:31.

### A Recombinant AAV Construct Comprising a Polynucleotide

The invention provides a recombinant AAV construct which comprises a polynucleotide comprising a Factor VIII nucleotide sequence, wherein the Factor VIII nucleotide sequence encodes a Factor VIII polypeptide comprising a Factor VIII amino acid sequence. The polynucleotide may be any polynucleotide of the invention. The Factor VIII nucleotide sequence may be any Factor VIII nucleotide sequence of the invention. The Factor VIII polypeptide may be any Factor VIII polypeptide of the invention.

The recombinant AAV construct may be less than 4900, less than 4850, less than 4800, or less than 4750 nucleotides in length. The recombinant AAV construct may be between 4700 and 4900, between 4700 and 4850, between 4700 and 4800, between 4700 and 4750 or around 4713 nucleotides in length. The recombinant AAV construct may be around 4845 nucleotides in length.

The recombinant AAV construct may be between 4850 and 4900, or around 4845 nucleotides in length, and may optionally comprise a polynucleotide comprising a Factor VIII nucleotide sequence of less than 4318 nucleotides in length. Optionally, the Factor VIII nucleotide sequence may be between 4318 and 4046, between 4318 and 4070, between 4264 and 4127, between 4210 and 4151 or around 4182 nucleotides in length.

The recombinant AAV construct may be between 4700 and 4850, between 4700 and 4800, between 4700 and 4750 or around 4713 nucleotides in length, and may optionally comprise a polynucleotide comprising a Factor VIII nucleotide sequence of less than 4318 nucleotides in length. Optionally, the Factor VIII nucleotide sequence may be between 4318 and 4046, between 4264 and 4070, between 4264 and 4103, between 4255 and 4127, between 4246 and 4130, between 4237 and 4154, between 4228 and 4157, between 4219 and 4166, between 4210 and 4169, between 4201 and 4172, between 4192 and 4175 or around 4182 nucleotides in length. Optionally, the Factor VIII nucleotide sequence may be around 4182 nucleotides in length.

Optionally, the recombinant AAV construct may be around 4713 nucleotides in length and the Factor VIII nucleotide sequence may be between 4228 and 4157, between 4219 and 4166, between 4210 and 4169, between 4201 and 4172, between 4192 and 4175 or around 4131, around 4134, around 4155, around 4158, around 4161, around 4167, around 4173 or around 4182 nucleotides in length. Optionally, the recombinant AAV construct may be around 4713 nucleotides in length and the Factor VIII nucleotide sequence may be 4131, 4134, 4155, 4158, 4161, 4167, 4173 or 4182 nucleotides in length. Optionally the recombinant AAV construct may be 4713 nucleotides in length and the Factor VIII nucleotide sequence may be SEQ ID NO:27.

In some cases, reference to the lengths of the Factor VIII nucleotide sequences in the above passages in the context of the recombinant AAV construct does not include a ‘stop’ codon—Factor VIII nucleotide sequences which include a stop codon would be three nucleotides longer than the nucleotide sequences recited above. In some embodiments, the Factor VIII nucleotide sequences may comprise a stop codon.

The recombinant AAV construct comprise a Factor VIII nucleotide sequence operably linked to a transcriptional regulatory element (TRE) and/or a polyA sequence.

The recombinant AAV construct may further comprise a transcription regulatory element (TRE). The transcription regulatory element may comprise a liver-specific promoter. The transcription regulatory element may be fewer than 270 nucleotides in length. The TRE may optionally comprise or consist of a sequence which is at least 90%, at least 95%, at least 96%, at least 97%, at least 98%, at least 99% or 100% identical to the sequence set forth in SEQ ID NO: 13. The present inventors have surprisingly determined that a substantial number of nucleotides from the known HLP2 TRE can be deleted or modified without significantly impacting the efficacy of the TRE. In particular, the present inventors have surprisingly found that the short transcription regulatory element FRE 72 is of comparable efficacy (i.e. at least 50% or better activity by comparison) with the HLP2 TRE despite being of considerably shorter length. Optionally, the transcription regulatory element is shorter than 200 nucleotides, optionally shorter than 150 nucleotides, optionally shorter than 125 nucleotides. Optionally, the transcription regulatory element is at least 85 nucleotides in length, optionally at least 100 nucleotides in length, optionally at least 110 nucleotides in length. Optionally, the transcriptional regulatory element may comprise or consist of a sequence which is at least 90%, at least 95%, at least 96%, at least 97%, at least 98%, at least 99% or 100% identical to the sequence set forth in SEQ ID NO:14.

The recombinant AAV construct may further comprise a nucleotide sequence encoding a signal peptide. The signal peptide may be a wild-type Factor VIII signal peptide. The signal peptide may comprise the wild-type FVIII signal peptide set forth in SEQ ID NO: 15. Optionally, the nucleotide sequence encoding the signal peptide is at least 90%, at least 92%, at least 95%, at least 98%, at least 99% or 100% identical to SEQ ID NOs: 16 or 17. Optionally, the nucleotide sequence encoding the signal peptide comprises SEQ ID NOs: 16 or 17.

Optionally, the signal peptide is not a wild-type Factor VIII signal peptide. The signal peptide may comprise a sequence that is at least 90%, at least 92%, at least 95%, at least 98%, at least 99% or 100% identical to the sequence of SEQ ID NOs: 18 or 20. The signal peptide may comprise a sequence that is at least 90%, at least 92%, at least 95%, at least 98%, at least 99% or 100% identical to SEQ ID NO: 18. The signal peptide may comprise a sequence that is at least 90%, at least 92%, at least 95%, at least 98%, at least 99% or 100% identical to SEQ ID NO: 20. The signal peptide may comprise SEQ ID NO: 18. The signal peptide may comprise SEQ ID NO: 20.

Preferably, the nucleotide sequence encoding the signal peptide is fewer than 57 nucleotides in length. More preferably, the nucleotide sequence encoding the signal peptide is around 54 nucleotides in length. The nucleotide sequence encoding the signal peptide may be around 54 nucleotides in length and encode the sequence of SEQ ID NO: 18. The nucleotide sequence encoding the signal peptide may be around 54 nucleotides in length and encode the sequence of SEQ ID NO: 20. Optionally, the nucleotide sequence encoding the signal peptide may be codon optimised. A nucleotide sequence encoding the signal peptide set forth in SEQ ID NO: 18 has the sequence of SEQ ID NO: 19. A nucleotide sequence encoding the signal peptide set forth in SEQ ID NO: 20 has the sequence of SEQ ID NO: 21. Optionally, the nucleotide sequence encoding the signal peptide is at least 90%, at least 92%, at least 95%, at least 98%, at least 99% or 100% identical to SEQ ID NOs: 19 or 21. Optionally, the nucleotide sequence encoding the signal peptide comprises SEQ ID NOs: 19 or 21.

The recombinant AAV construct may further comprise a polyA nucleotide sequence. The polyA nucleotide sequence may be synthetic. The polyA nucleotide sequence may be fewer than 50 nucleotides in length. The polyA nucleotide sequence may be between 16 and 50 nucleotides in length. The polyA nucleotide sequence may be around 49 nucleotides in length. The polyA nucleotide sequence may comprises the nucleotide sequence of SEQ ID NO: 22.

The recombinant AAV construct may further comprise one or two ITR(s). The or each ITR may be a wild-type ITR. Optionally, a recombinant AAV construct comprises ITR sequences which are derived from AAV1, AAV2, AAV4 and/or AAV6. The or each ITR may be an AAV2 ITR. The nucleotide sequence of the or each ITR may be fewer than 157, or fewer than 154 nucleotides in length. The nucleotide sequence of the or each ITR may be around 145 nucleotides in length. The nucleotide sequence of a 5′ ITR may comprise the nucleotide sequence of SEQ ID NO: 23. The nucleotide sequence of a 3′ ITR may comprise the nucleotide sequence of SEQ ID NO:24.

The recombinant AAV construct may be single-stranded. The recombinant AAV construct may be an AAV genome.

The Factor VIII nucleotide sequence encoding the Factor VIII amino acid sequence may comprise the sequence of SEQ ID NO: 27 and the nucleotide sequence encoding the signal peptide may comprise SEQ ID NO: 19. The recombinant AAV construct may comprise a transcriptional regulatory element which is a liver-specific promoter, and the liver-specific promoter may comprise the nucleotide sequence of SEQ ID NO: 13. The recombinant AAV construct may comprise two ITRs and a polyA nucleotide sequence, wherein the nucleotide sequence of the 5′ ITR is SEQ ID NO: 23 and the sequence of the 3′ ITR is SEQ ID NO:24, and the polyA nucleotide sequence comprises the nucleotide sequence of SEQ ID NO: 22. The AAV construct may have the sequence SEQ ID NO: 27.

### A Viral Particle Comprising the Construct

The invention also provides an AAV viral particle comprising the recombinant AAV construct of the invention.

The invention further provides a viral particle comprising a recombinant genome comprising polynucleotides of the invention. For the purposes of the present invention, the term “viral particle” refers to all or part of a virion. For example, the viral particle comprises a recombinant genome and may further comprise a capsid. The viral particle may be a gene therapy vector. Herein, the terms “viral particle” and “vector” are used interchangeably. For the purpose of the present application, a “gene therapy” vector is a viral particle that can be used in gene therapy, i.e. a viral particle that comprises all the required functional elements to express a transgene, such as a Factor IX nucleotide sequence, in a host cell after administration.

Suitable viral particles include a parvovirus, a retrovirus, a lentivirus or a herpes simplex virus. The parvovirus may be an adeno-associated virus (AAV). The viral particle is preferably a recombinant adeno-associated viral (AAV) vector or a lentiviral vector. More preferably, the viral particle is an AAV viral particle. The terms AAV and rAAV are used interchangeably herein.

The genomic organization of all known AAV serotypes is very similar. The genome of AAV is a linear, single-stranded DNA molecule that is less than about 5,000 nucleotides in length. Inverted terminal repeats (ITRs) flank the unique coding nucleotide sequences for the non-structural replication (Rep) proteins and the structural (VP) proteins. The VP proteins (VP1, -2 and -3) form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. Following wild-type (wt) AAV infection in mammalian cells the Rep genes (i.e. encoding Rep78 and Rep52 proteins) are expressed from the P5 promoter and the P19 promoter, respectively, and both Rep proteins have a function in the replication of the viral genome. A splicing event in the Rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

AAV sequences that may be used in the present invention for the production of AAV vectors can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chiorini et al, 1997; Srivastava et al, 1983; Chiorini et al, 1999; Rutledge et al, 1998; and Wu et al, 2000. AAV serotype 1, 2, 3, 3B, 4, 5, 6, 7, 8, 9, 10, 11 or 12 may be used in the present invention. The sequences from the AAV serotypes may be mutated or engineered when being used in the production of gene therapy vectors.

Optionally, an AAV vector comprises ITR sequences which are derived from AAV1, AAV2, AAV4 and/or AAV6. Preferably the ITR sequences are AAV2 ITR sequences. Herein, the term AAVx/y refers to a viral particle that comprises some components from AAVx (wherein x is a AAV serotype number) and some components from AAVy (wherein y is the number of the same or different serotype). For example, an AAV2/8 vector may comprise a portion of a viral genome, including the ITRs, from an AAV2 strain, and a capsid derived from an AAV8 strain.

In an embodiment, the viral particle is an AAV viral particle comprising a capsid. AAV capsids are generally formed from three proteins, VP1, VP2 and VP3. The amino acid sequence of VP1 comprises the sequence of VP2. The portion of VP1 which does not form part of VP2 is referred to as VP1unique or VP1U. The amino acid sequence of VP2 comprises the sequence of VP3. The portion of VP2 which does not form part of VP3 is referred to as VP2unique or VP2U. The viral particle may comprise a capsid. The capsid may be selected from the group consisting of:
(i) a capsid comprising a sequence which is at least 96%, at least 98%, at least 99%, at least 99.5%, at least 99.8% or 100% identical to SEQ ID NO: 25;
(ii) a capsid comprising a sequence which is at least 96%, at least 98%, at least 99%, at least 99.5%, at least 99.8%, or 100% identical to SEQ ID NO: 26;
(iii) liver-tropic capsid; and
(iv) an AAV5 capsid.

The capsid may be selected from the group consisting of;
(i) a capsid comprising a sequence which is at least 96%, at least 98%, at least 99%, at least 99.5%, at least 99.8% or 100% identical to SEQ ID NO: 47;
(ii) a capsid comprising a sequence which is at least 96%, at least 98%, at least 99%, at least 99.5%, at least 99.8% or 100% identical to SEQ ID NO: 48; and
(iii) an AAV6 capsid.

A viral particle of the invention may be a “hybrid” particle in which the viral ITRs and viral capsid are from different parvoviruses, such as different AAV serotypes. Preferably, the viral ITRs and capsid are from different serotypes of AAV, in which case such viral particles are known as transcapsidated or pseudotyped. Likewise, the parvovirus may have a “chimeric” capsid (e. g., containing sequences from different parvoviruses, preferably different AAV serotypes) or a “targeted” capsid (e.g., a directed tropism).

### Compositions, Methods and Uses

In a further aspect of the invention, there is provided a composition comprising the polynucleotide or construct/viral particle of the invention and a pharmaceutically acceptable excipient.

The pharmaceutically acceptable excipients may comprise carriers, diluents and/or other medicinal agents, pharmaceutical agents or adjuvants, etc. Optionally, the pharmaceutically acceptable excipients comprise saline solution. Optionally, the pharmaceutically acceptable excipients comprise human serum albumin.

The invention further provides a polynucleotide, construct/viral particle or composition of the invention for use in a method of treatment. Optionally the method of treatment comprises administering an effective amount of the polynucleotide or construct/viral particle of the invention to a patient.

The invention further provides use of the polynucleotide, construct/viral particle or composition of the invention in the manufacture of a medicament for use in a method of treatment. Optionally the method of treatment comprises administering an effective amount of the polynucleotide or construct/viral particle of the invention to a patient.

Optionally the method of treatment is a gene therapy. A “gene therapy” involves administering a construct/viral particle of the invention that is capable of expressing a transgene (such as a Factor VIII nucleotide sequence) in the host to which it is administered.

Optionally, the method of treatment is a method of treating a coagulopathy such as haemophilia (for example haemophilia A or B) or Von Willebrands' disease. Preferably, the coagulopathy is characterised by increased bleeding and/or reduced clotting. Optionally, the method of treatment is a method of treating haemophilia, for example haemophilia A. In some embodiments, the patient is a patient suffering from haemophilia A. Optionally the patient has antibodies or inhibitors to Factor VIII. Optionally, the polynucleotide and/or construct/viral particle is administered intravenously. Optionally, the polynucleotide and/or construct/viral particle is for administration only once (i.e. a single dose) to a patient.

Optionally, the method of the invention may further comprise a step of determining whether the patient has been partially or fully treated (e.g. determining that the patient has been partially or fully treated for the symptoms of haemophilia A) by administration of the gene therapy vector. Partially or fully treating haemophilia A may refer to improving the clotting of a patient suffering from haemophilia A, and reducing the risk of an uncontrolled bleeding event. Partially or fully treating haemophilia A may refer to reducing the number and/or frequency of uncontrolled bleeding events or internal bleeding e.g. in joints. A patient who is partially or fully treated may suffer fewer uncontrolled bleeding events per year. When haemophilia A is “treated” in the above method, this means that one or more symptoms of haemophilia are ameliorated. It does not mean that the symptoms of haemophilia are completely remedied so that they are no longer present in the patient, although in some methods, this may be the case. The method of treatment may result in one or more of the symptoms of haemophilia A being less severe than before treatment. Partially or fully treating haemophilia A may refer to increasing the amount or activity of FVIII present in the plasma of the patient. Optionally, relative to the situation pre-administration, the method of treatment results in an increase in the amount/concentration of circulating Factor VIII in the blood of the patient, and/or the overall level of Factor VIII activity detectable within a given volume of blood of the patient, and/or the specific activity (activity per amount of Factor VIII protein) of the Factor VIII in the blood of the patient. Partially treating haemophilia may refer to converting a patient with severe (<1% normal blood clotting factor activity) or moderately severe haemophilia (<2% normal blood clotting factor activity) to a patient with mild haemophilia (5-40% normal blood clotting factor activity). Fully treating haemophilia may refer to increasing the blood clotting factor activity of a patient with severe, moderately severe or mild haemophilia to within the normal range (50%-150% normal blood clotting factor activity).

A patient who is partially or fully treated may require a lower dose of Factor VIII to be administered. Optionally, a patient who is partially or fully treated may not require the on-going administration of FVIII. Optionally, the method may further comprise a step of assessing whether a patient who has been partially or fully treated for haemophilia no longer needs ongoing treatment or needs a reduced level of ongoing treatment with Factor VIII, and adjusting the patient's treatment regimen appropriately, e.g. to reduce the dose or dose frequency of the Factor VIII that is to be administered, or to halt the administration of the Factor VIII.

A “therapeutically effective amount” refers to an amount effective, at dosages and for periods of time necessary, to achieve the desired therapeutic result, such as raising the level of functional factor VIII in a subject (so as to lead to functional factor VIII production at a level sufficient to ameliorate the symptoms of haemophilia A).

Optionally, the construct/viral particle is administered at a dose of less than 1×10¹¹, less than 1×10¹², less than 5×10¹², less than 2×10¹², less than 1.5×10¹², less than 3×10¹², less than 1×10¹³, less than 2×10¹³, or less than 3×10¹³ construct genomes per kg of weight of patient. Optionally, the polynucleotide, viral particle or composition is administered in a dose of at least 4.5×10¹¹, or between 4.5×10¹¹ and 1×10¹² construct genomes per kg of weight of patient (vg/kg). Optionally, the polynucleotide, viral particle or composition is administered in a dose of less than 5×10¹¹ vg/kg. Optionally, the polynucleotide, viral particle or composition is administered in a dose of between 4.5×10¹¹ vg/kg and 5×10¹¹ vg/kg, or between 4.5×10¹¹ vg/kg and 4.9×10¹¹ vg/kg. Optionally, the polynucleotide or viral particle that is administered is produced from a mammalian cell, and/or possesses characteristics which result from use of mammalian viral construct production cells and distinguish from constructs produced in insect viral construct production cells (e.g. baculovirus system).

Optionally, administration of a given dose of constructs/viral particles—quantified in terms of the number of construct genomes—is achieved using qPCR to titrate construct genomes.

Methods of performing qPCR are known to those of skill in the art. Using a real-time PCR cycler and DNA-binding dye such as SYBR Green (ThermoFisher Scientific) the amplification of nascent double-stranded amplicons can be detected in real time. Known quantities of the qPCR template genetic material (e.g. the promoter region) may then be serially diluted to create a standard curve, and sample construct genome titre interpolated from the standard curve.

### EXAMPLES

### Example 1—General Materials and Methods

### FVIII Constructs

The FVIII-SQ polypeptide comprises amino acids corresponding to amino acids 1-743 and 1638-2332 of SEQ ID NO:1) (Lind et al. 1995. Eur J Biochem 232, 19-27) (SEQ ID NO:3).

An internally-truncated human FVIII polypeptide (FVIII-96-106) comprises a deletion of amino acids corresponding to amino acids 732-1669 of SEQ ID NO: 1), and comprises amino acids corresponding to amino acids 1-731 and 1670-2332 of SEQ ID NO: 1 (SEQ ID NO:7).

### Generation of AAV Vectors

AAV particles were produced by triple plasmid transfection of HEK293T cells with plasmids encoding the AAV Rep and Cap functions; adenoviral helper functions; and the recombinant genome containing the FVIII expression cassette flanked by AAV2 ITRs. Cell pellet and supernatant were harvested 72 hours post-transfection and AAV particles purified by affinity chromatography using resins such as POROS Capture Select and AVB Sepharose. AAV was then dialysed into PBS overnight, stored at 4° C. and titred by qPCR.

### FVIII Chromogenic Activity Assay

The Biophen FVIII:C chromogenic assay (Hyphen BioMed, ref 221406) measures the cofactor activity of FVIII (FVIII:C).

Through thrombin activation, the FVIII:C polypeptide forms a complex with human Factor IXa, phospholipids and calcium. Under these conditions, Factor X, provided in this assay at a specific concentration and in excess, is converted into Factor Xa (activated). This Factor Xa produced is directly proportional to FVIII:C, the limiting factor. Factor Xa is directly measured by a chromogenic substrate, Sxa-11. Factor Xa cleaves the chromogenic substrate and releases pNA. Production of pNA is proportional to Factor Xa activity, which is directly related to FVIII:C activity. The level of pNA released can determined by measuring colour development at 405 nm, and this is relative to the amount of the Factor Xa polypeptide generated by Factor VIII:C in the sample, which is proportional to the activity of FVIII:C in the sample.

The assay is performed according to manufacturer's instructions. Briefly, to a microplate well, preincubated at 37° C., 50 μl of calibrator plasmas, diluted (in reagent R4) test plasmas or cell supernatants/lysates or controls, is added, followed by 50 μl each of reagent R1 and R2, which are reconstituted with 6 mL of distilled water and prewarmed to 37° C. After mixing, these components form a 150 μl reaction that is allowed to incubate for 5 min at 37° C. Subsequently, the reaction is supplemented with reagent R3, which is itself resuspended in 6 mL of distilled water and prewarmed to 37° C., and the 200 μL mix is allowed to incubate for a further 5 min at 37° C. The reaction is stopped by adding 50 μl of 20% acetic acid or citric acid (20 g/l), before measuring the absorbance of the resulting 250 μl mixture at 405 nm.

### Reagents:

R1—Human Factor X, lyophilized in presence of a fibrin polymerization inhibitor.

R2—Activation Reagent—Factor IXa (human), at a constant and optimized concentration, containing human thrombin, calcium and synthetic phospholipids, lyophilized.

R3—SXa-11—Chromogenic substrate, specific for Factor Xa, lyophilized, with a thrombin inhibitor.

R4—Tris-BSA Buffer. Contains 1% BSA, PEG, FVIII:C Stabilizer and sodium azide (0.9 g/L).

In relation to the readout from the chromogenic activity assay, the “% FVIII activity” (also referred to as “% FVIII:C”) is the “% normal” which means, for example in the context of expressing a FVIII expression cassette in HuH-7 cells, that relative to a human plasma sample having 100% FVIII activity, the FVIII activity detected in a supernatant following expression of a FVIII expression cassette in HuH-7 cells is a specified % of the FVIII activity detected in said human plasma sample having 100% FVIII activity.

### FVIII Sandwich ELISA Antigen Assay

The Asserachrom VIII:Ag kit (Stago Diagnostica, ref 00280) is an antigenic assay for quantification of FVIII in plasma by enzyme-linked immunosorbent assay (ELISA). FVIII in assayed samples is captured by a mouse-monoclonal anti-human VIII:Ag antibody, pre-coating the walls of a plastic microplate well. Following sufficient incubation and washing to reduce non-specific binding, mouse anti-human FVIII antibodies, coupled to peroxidase, bind to the remaining free antigenic determinants of the captured FVIII. The bound peroxidase is then revealed by TMB substrate. The colour development induced by TMB is halted by the addition of a strong acid. The intensity of the colour development is directly proportional to the FVIII concentration in the assayed sample, determined by measuring the absorbance at 450 nm.

The readout from this assay may be expressed as “% normal” which means, for example in the context of expressing FVIII constructs in mice, that relative to a human plasma sample having 100% FVIII activity, the number of FVIII molecules (strictly, epitopes) detected in a mouse plasma sample is a specified % of the number of FVIII molecules/epitopes detected in said human plasma sample having 100% FVIII activity.

In both the activity and antigen assays described above, FVIII (activity or antigen levels) is quantified in mouse, or human cell supernatant, samples using the manufacturer recommended or included lyophilized human plasma samples of known FVIII activity or antigen (as appropriate), calibrated against the WHO International Standard (NIBSC code 07/316).

### Example 2—In Vitro Evaluation of Relative Specific Activity of FVIII Substitution Mutation Variants

In silico modelling was used to predict single amino acid substitutions, and pairs of cysteine substitutions, on non-surface-exposed inter-domain surfaces, which might increase the stability of FVIII. However, any impact on stability does not necessarily equate to a beneficial effect on activity, and in some cases increased stability would have a deleterious effect, e.g. a substitution increasing stability but not allowing the necessary FVIII domain rearrangement into an active form. Numerous such substitution variants were tested in vitro as described below.

Codon-optimised nucleotide sequences encoding FVIII-SQ variants comprising one or more such substitutions were gene-synthesised and cloned into the commercially available expression vector pcDNA5-FRT (Invitrogen). Plasmid DNA was transfected into expi293 suspension cells (Invitrogen) in 96 Deepwell plates according to the manufacturer's protocol, and incubated in a humidified 8% CO₂ incubator at 37° C. shaking at 400 rpm for 5 days. Cell cultures were centrifuged at 1000×g for 5 min at 4° C. and the supernatants filtered through 0.2 μl filter for immediate testing in chromogenic assay and antigen quantification as described in Example 1.

Following blank subtraction, the FVIII activity readout from the chromogenic assay was divided by the FVIII antigen quantification (ELISA) readout to obtain the specific activity (SA) value.

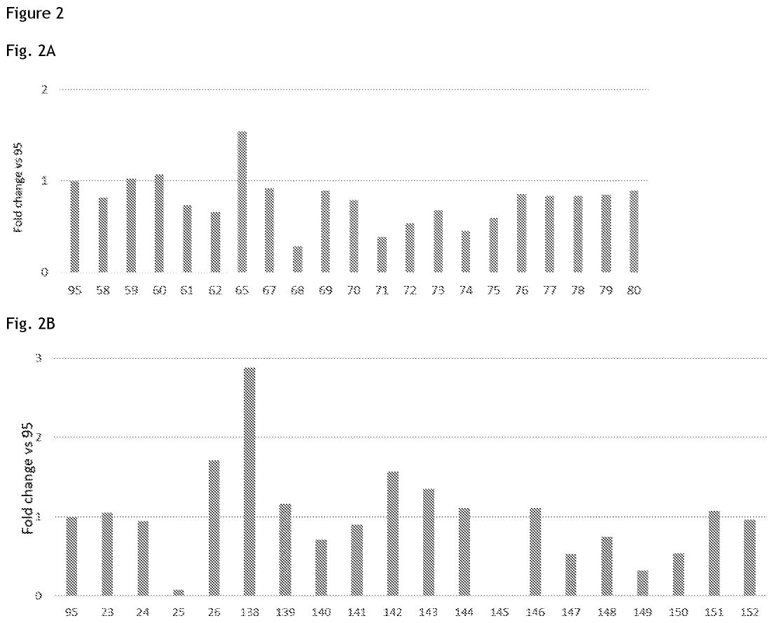

FIG. 2A shows, the fold-change in SA, relative to the FVIII-SQ (‘95’) control lacking any substitution mutations, for several different single amino-acid substitution variants, including a number of alternative substituted residues for each variant. The following table shows the identity of the substitutions corresponding to each of the numbered constructs. Variant 65 (H693W) exhibits an elevation in SA relative to 95.

| Construct no. | 58 | 59 | 60 | 61 | 62 | 65 | 67 | 68 |
|---|---|---|---|---|---|---|---|---|
| Position | L687 | | H693 | | | | N694 | |
| Substituted residue | R | W | R | I | K | W | E | W |

| Construct no. | 69 | 70 | 71 | 72 | 73 | 74 | 75 | 76 | 77 | 78 | 79 | 80 |
|---|---|---|---|---|---|---|---|---|---|---|---|---|
| Position | S695 | | | | | | | D696 | | | | |
| Substituted | R | I | L | M | F | W | Y | R | Q | F | W | K |
| residue | | | | | | | | | | | | |

In another experiment, the substitution M662W (called ‘26’) had demonstrated a marked increase in SA relative to ‘95’. For position M662 it was decided to screen the SA of all alternative substitutions. The results are shown in FIG. 2B, which also includes a double substitution, combining M662W with H693W (65 from FIG. 2A). Again see below table for the identity of the substitutions. Substitutions at position M662 with C and E appear to give some uplift in SA relative to 95. The greatest increase in SA was observed for the M662W+H693W combination.

| Construct no. | 23 | 24 | 25 | 26 | 138 | 139 | 140 | 141 | 142 | 143 |
|---|---|---|---|---|---|---|---|---|---|---|
| Position | M662 | | | | | | | | | |
| Substituted | N | Q | I | W | W + | A | R | D | C | E |
| residue | | | | | H693W | | | | | |

| Construct no. | 144 | 145 | 146 | 147 | 148 | 149 | 150 | 151 | 152 |
|---|---|---|---|---|---|---|---|---|---|
| Position | M662 | | | | | | | | |
| Substituted | G | P | S | Y | H | L | K | F | T |
| residue | | | | | | | | | |

### Example 3—In Vitro Evaluation of Relative Specific Activity of Additional FVIII Substitution Mutation Variants Predicted to Form Disulphide Bridges

Based on the in silico prediction work in Example 2, a number of pairs of cysteine substitutions were tested using the methodology described in Example 2.

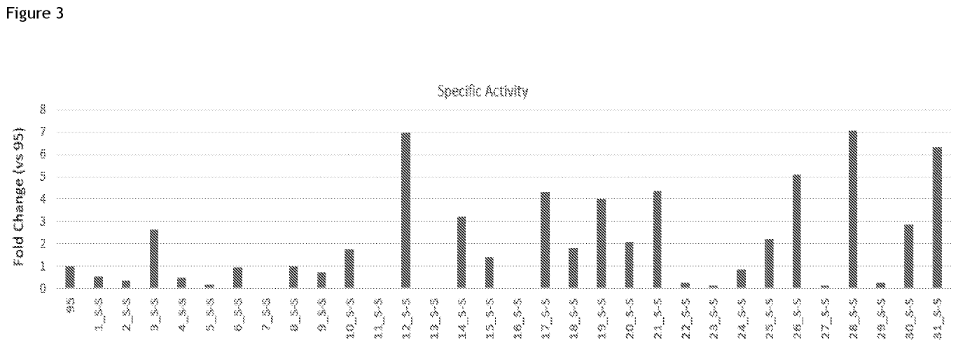

The results—again expressed as fold-change in specific activity relative to FVIII-SQ (95′) (SEQ ID NO:3)—are shown in FIG. 3. The below table indicates the substitution pairs corresponding to the construct numbers shown in FIG. 3. Several of the C-C substitution variants show several-fold increases in specific activity relative to control.

| Construct no. | 1_S-S | 2_S-S | 3_S-S | 4_S-S | 5_S-S | 6_S-S | 7_S-S | 8_S-S | 9_S-S |
|---|---|---|---|---|---|---|---|---|---|
| C substitution | S285C | E287C | S289C | T646C | D647C | K659C | Y664C | T667C | T667C |
| pair | E676C | F673C | N1977C | N1950C | Y1979C | M1823C | K1967C | S1788C | A1836C |
| Construct no. | 10_S-S | 11_S-S | 12_S-S | 13_S-S | 14_S-S | 15_S-S | 16_S-S | 17_S-S | 18_S-S |
| C substitution | T669C | F671C | L687C | W668C | I689C | F697C | G102C | A108C | T118C |
| pair | V1982C | Y1979C | A1800C | S710C | G1799C | S1949C | A1974C | Q2329C | N2172C |

| Construct no. | 19_S-S | 20_S-S | 21_S-S | 22_S-S | 23_S-S | 24_S-S | 25_S-S | 26_S-S | 27_S-S | 28_S-S |
|---|---|---|---|---|---|---|---|---|---|---|
| C | V137C | M147C | S149C | P264C | I291C | N280C | T667C | T669C | S268C | N684C |
| substitution | Y2332C | E1970C | E1969C | E1951C | S1955C | S524C | G1981C | Y1979C | P672C | S1791C |
| pair | | | | | | | | | | |

| Construct no. | 29_S-S | 30_S-S | 31_S-S |
|---|---|---|---|
| C substitution | G686C | L687C | S695C |
| pair | R1803C | R1803C | E1844C |

### Example 4—Confirmatory In Vitro Testing of Particular Promising Substitution Variants

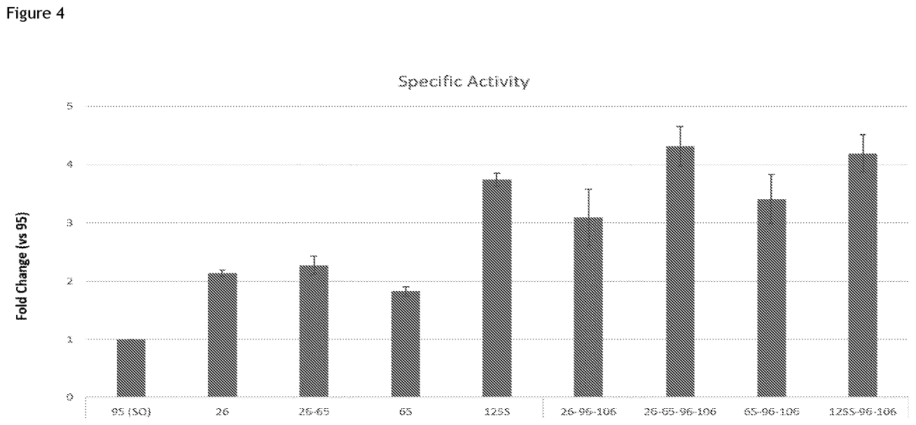

FIG. 4 confirms, for a subset of tested substitution variants of Examples 2 and 3, the fold-increase in SA relative to the FVIII-SQ (95′) control. The substitutions were evaluated in two FVIII ‘backgrounds’, the first being FVIII-SQ as in the above Examples:
FVIII-SQ-M662W (‘26’)
FVIII-SQ-H693W (‘65’)
FVIII-SQ-M662W-H693W (‘26-65’)
FVIII-SQ-L687C-A1800C (‘12SS’)

The second FVIII background differs from FVIII-SQ in that it contains a more extensive internal deletion encompassing, and extending on either side of, the B domain. The deleted region corresponds to amino acids positions 732-1669, and the variant protein is referred to as FVIII-(96-106).
FVIII-(96-106)-M662W (‘26-96-106’)
FVIII-(96-106)-H693W (‘65-96-106’)
FVIII-(96-106)-M662W-H693W (‘26-65-96-106’)
FVIII-(96-106)-L687C-A1800C (‘12SS-96-106’)

As FIG. 4 shows, each of the above subset of tested constructs showed an approximately 2 or more fold increase in specific activity relative to FVIII-SQ control (95).

### Example 5—In Vivo Evaluation of Internally-Deleted FVIII Transgene Construct Containing Stabilising Substitution Mutation

In this study, AAV8 vectors were made from the following constructs:
FRE72-SP5-FVIIICo19 (26-96-106)-SpA (SEQ ID NO:27)
FRE72-SP5-FVIIICo19-SQ-SpA (SEQ ID NO:32)

The above constructs contain the FRE72 promoter, signal peptide 5, and synthetic polyA. They differ in that one encodes FVIII-SQ, whilst the other encodes the shorter variant with the “96-106” internal deletion, as well as the substitution mutation “26” (=M662W).
Comparator FVIIIco-SQ

The above construct comprises a codon-optimised FVIII-SQ-encoding sequence with native FVIII signal peptide, SpA, and a liver-specific promoter. The construct has the sequence of SEQ ID NO: 34.

The AAV8-Comparator FVIIIco-SQ was respectively compared against AAV8-FRE72-SP5-FVIIICo19 (26-96-106)-SpA and AAV8-FRE72-SP5-FVIIICo19-SQ-SpA in separate experiments.

6-8 week old C7BL/6 Factor VIII-knockout (FVIII-KO) mice were intravenously injected with 2×10¹² vg/kg of one of each of the above vectors. All AAV8 vectors for this study were titered at the same time by qPCR method.

At 6 weeks post injection, between 100 and 200 μl of blood was collected from each mouse by retro-orbital puncture with non-heparinised blue capillary tubes on citrate (1:10 dilution). Plasma was prepared by centrifugation for 20 min at 4000 rpm.

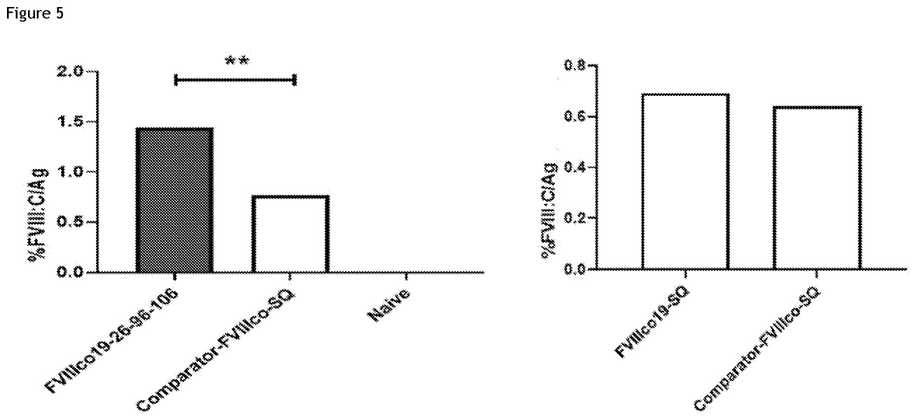

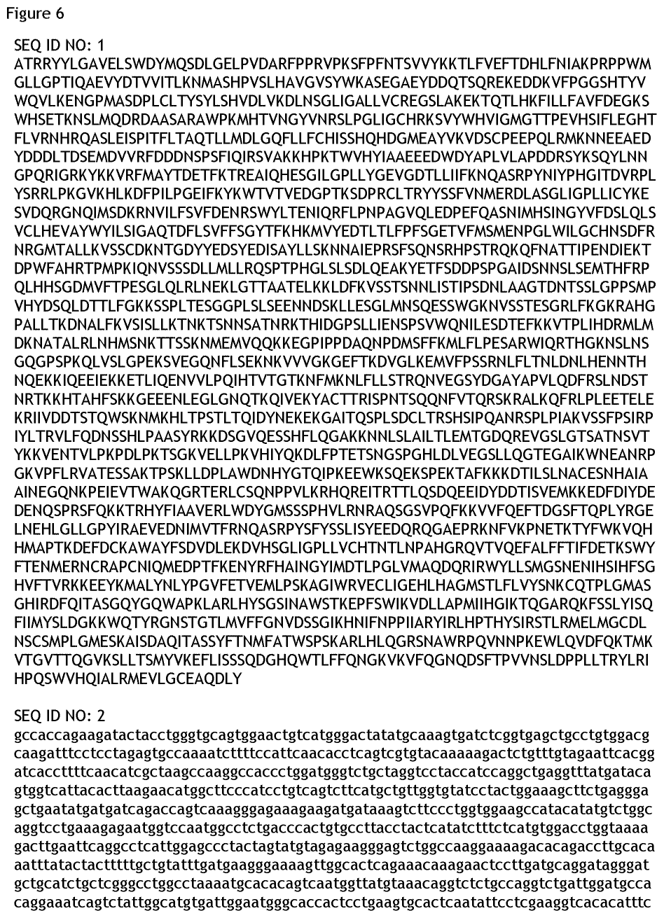

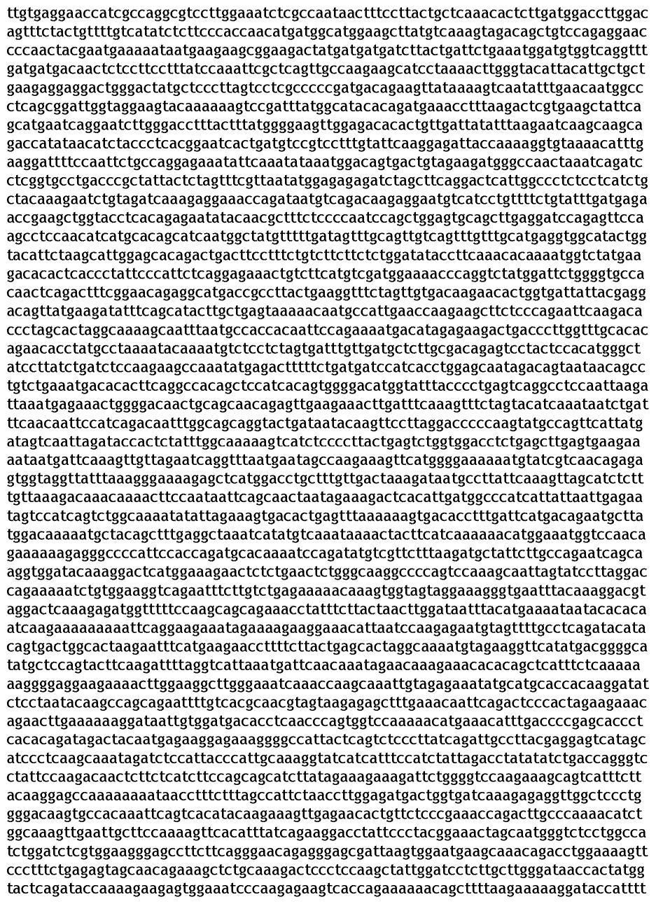

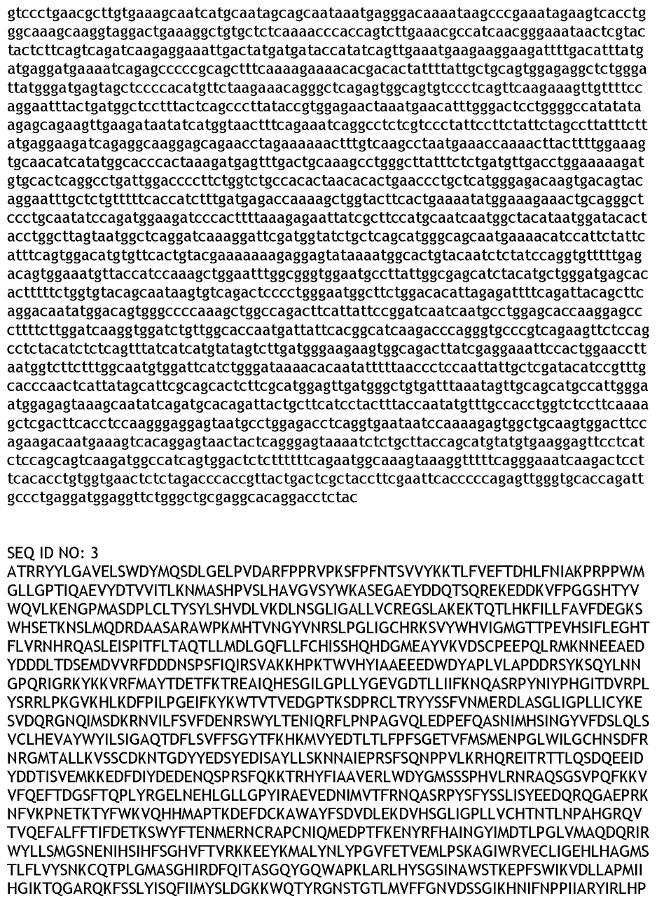

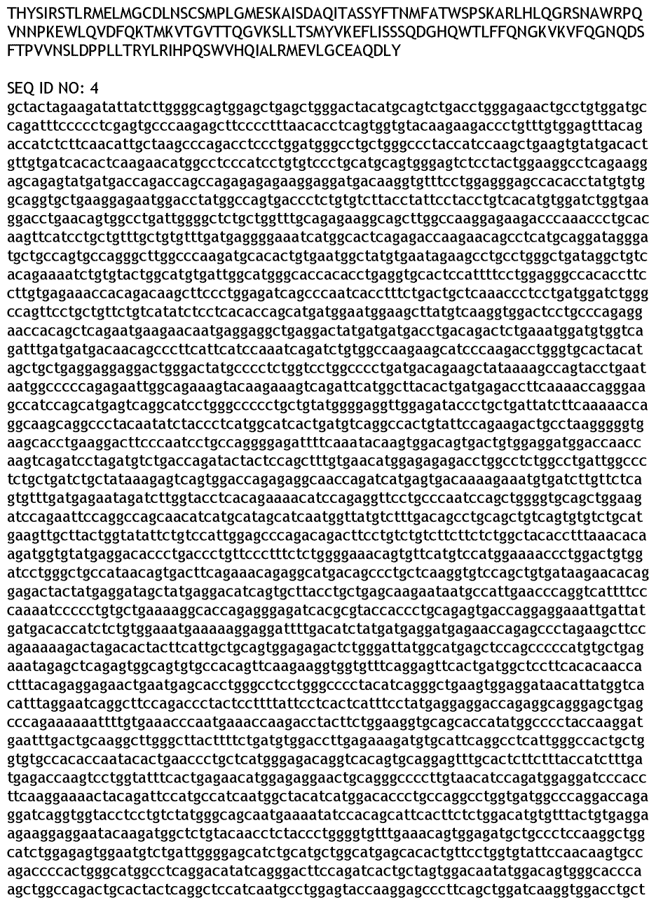

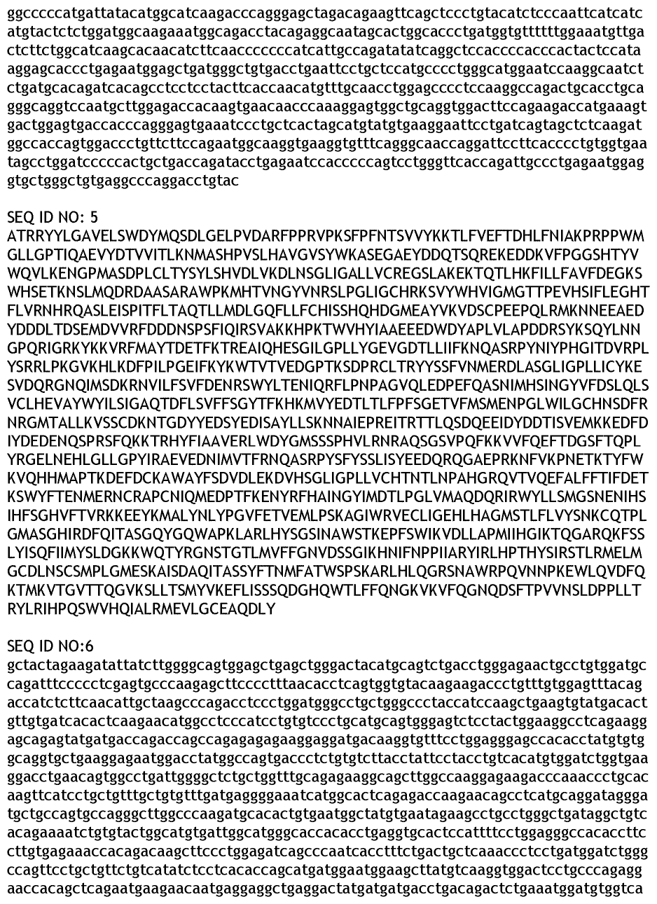

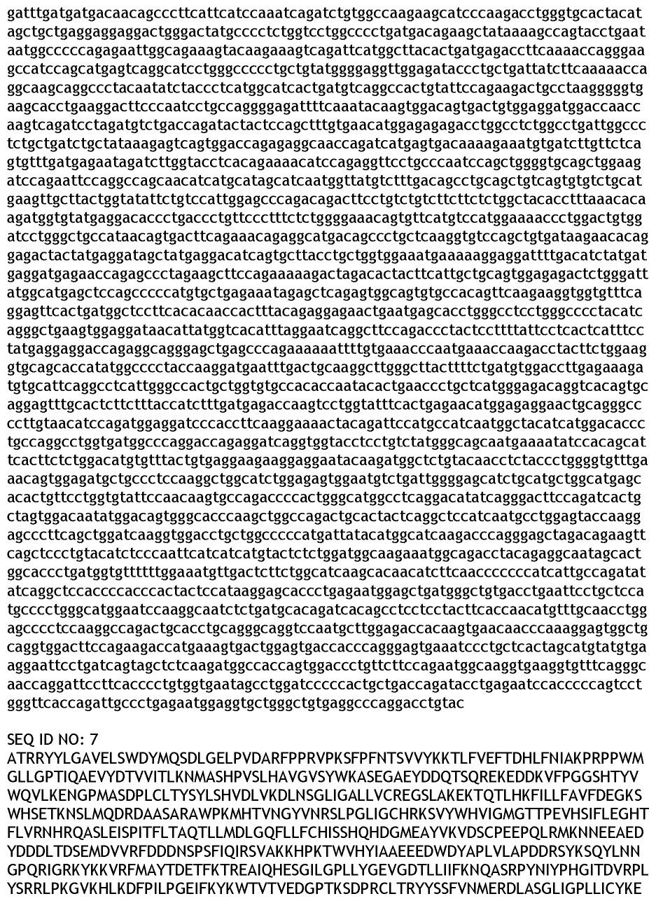

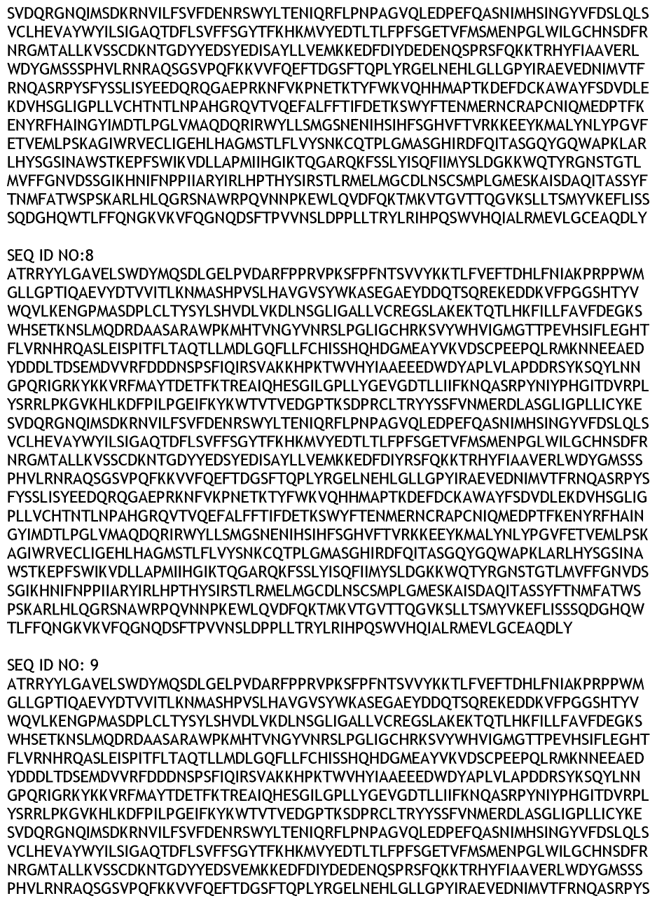

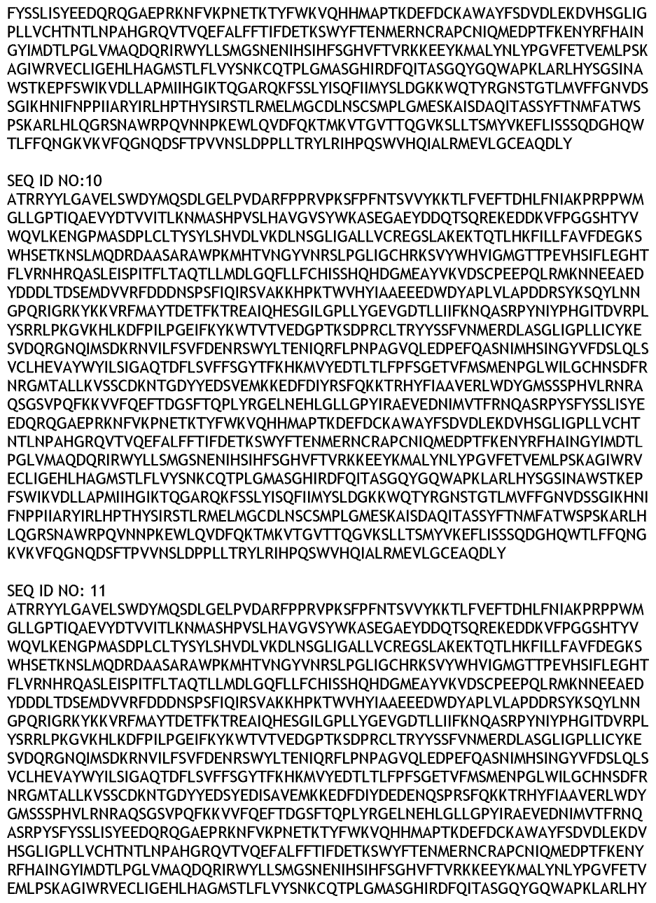

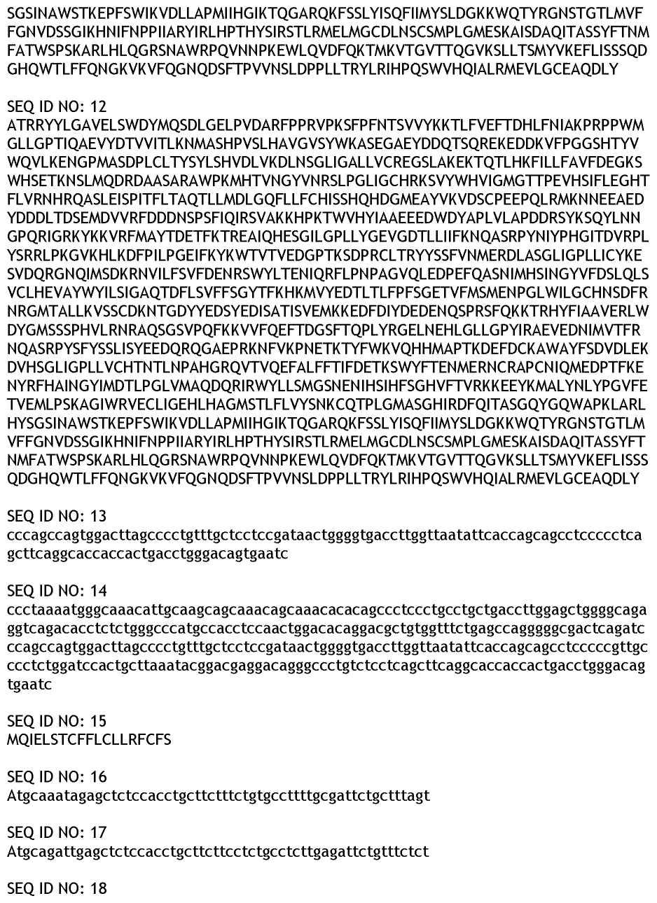

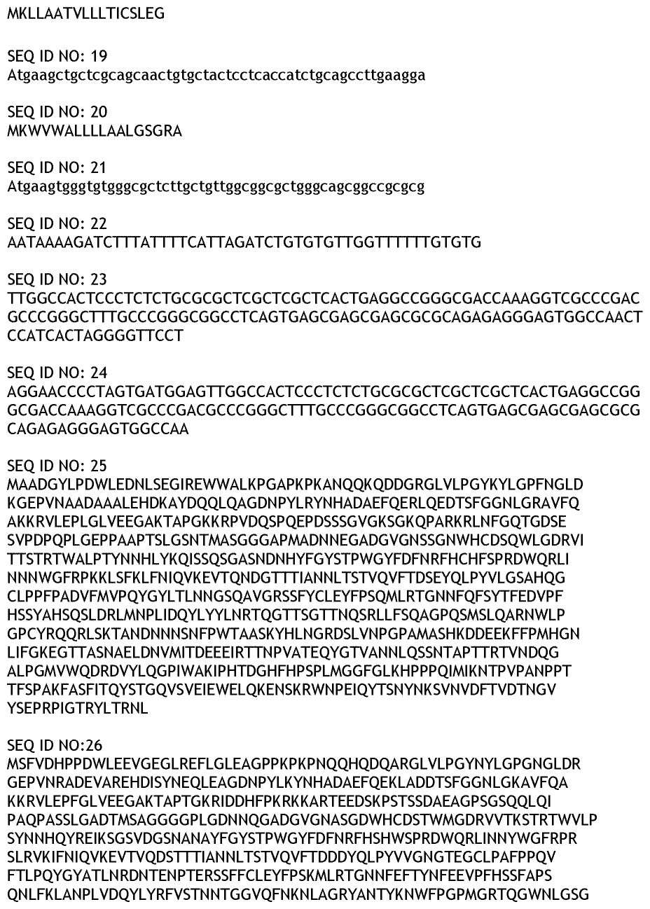

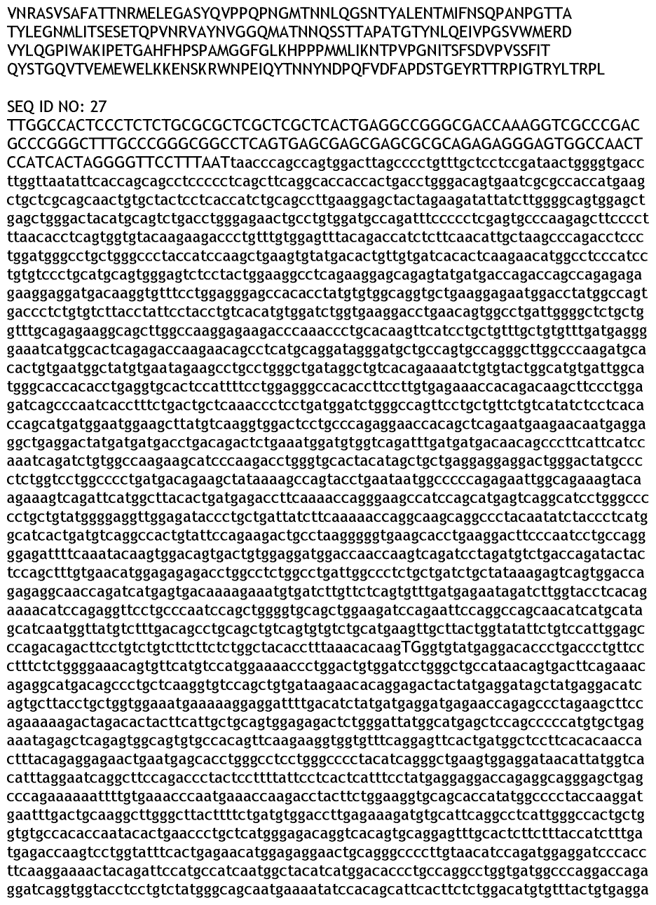

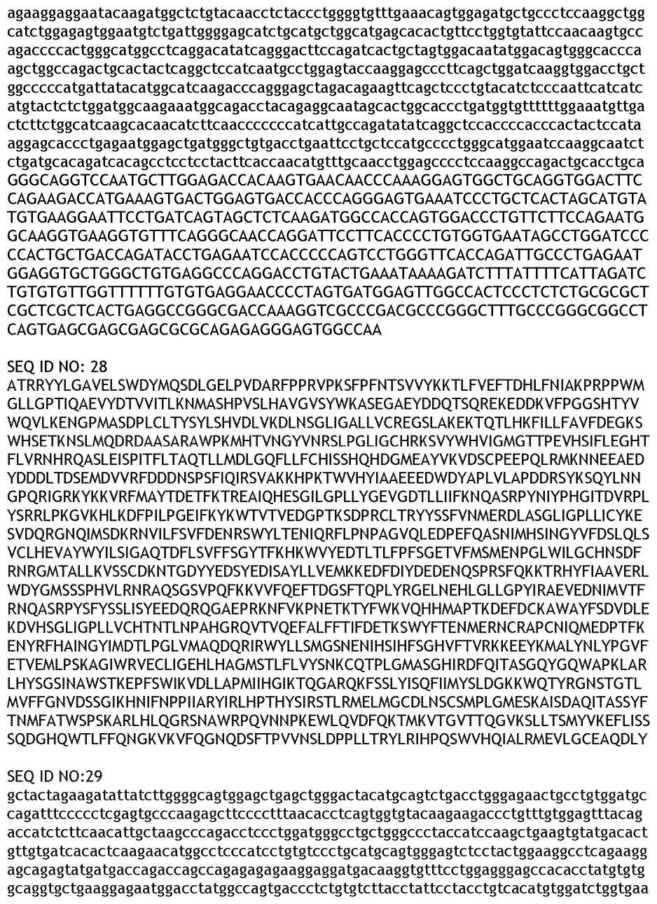

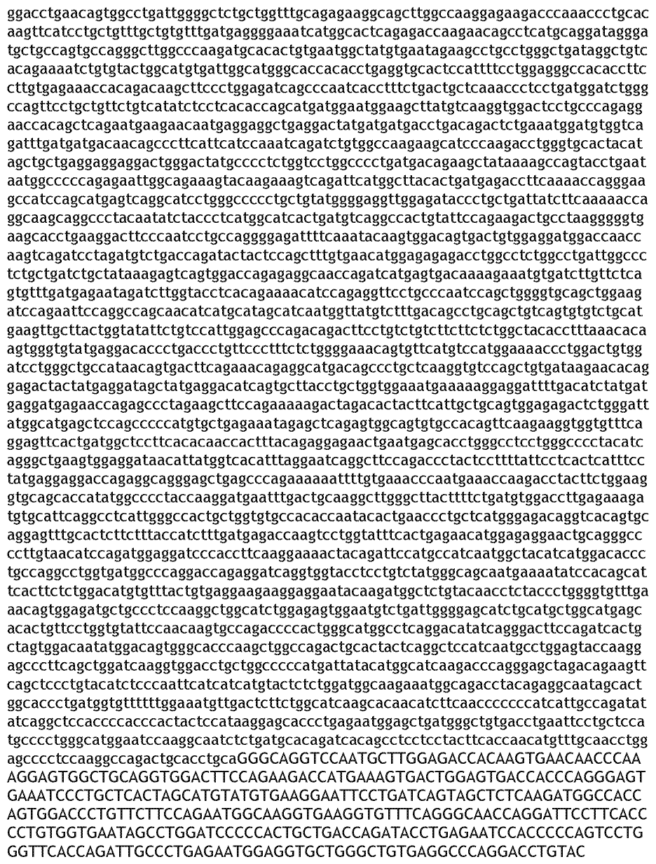

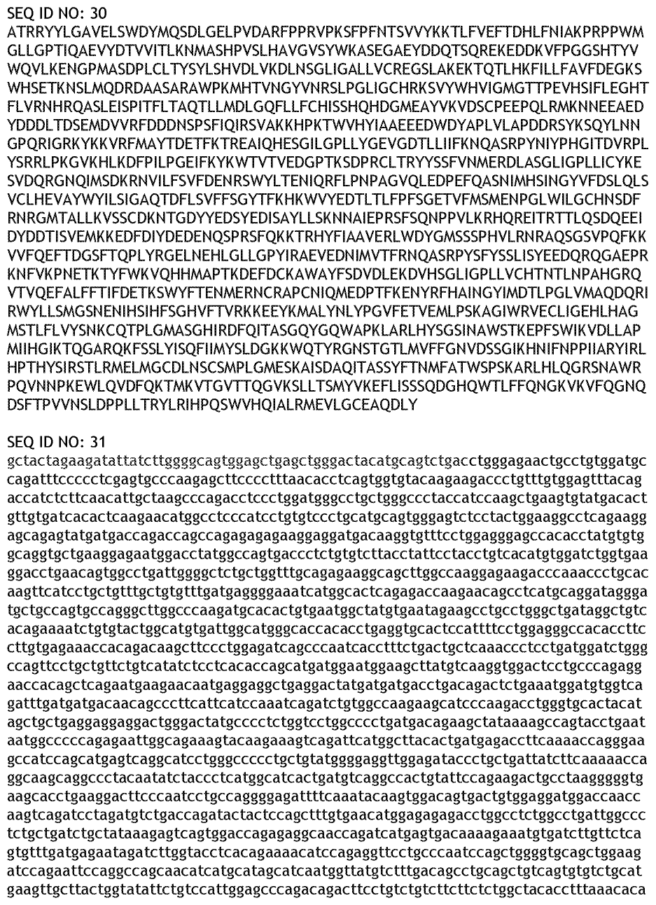

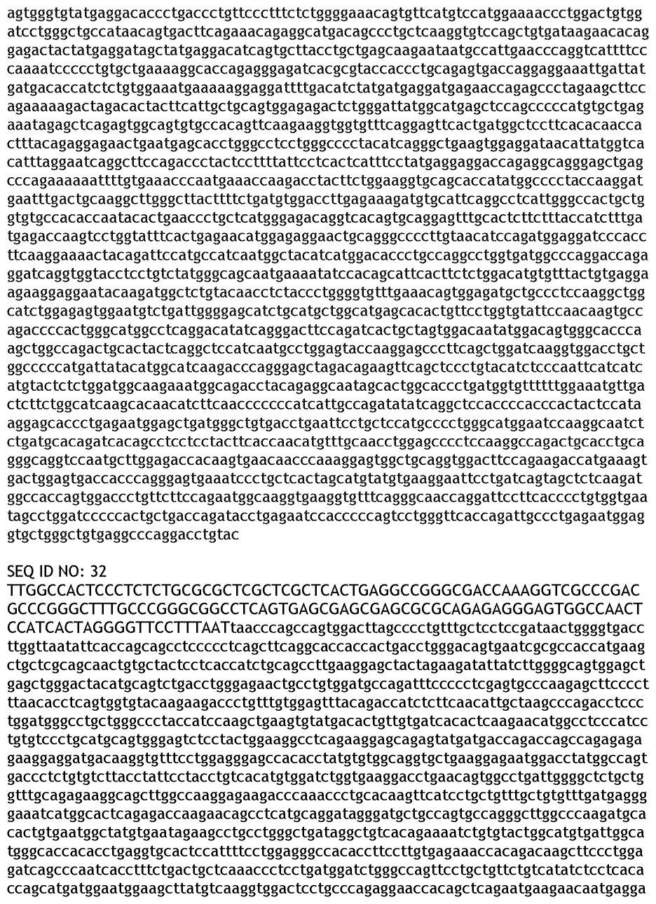

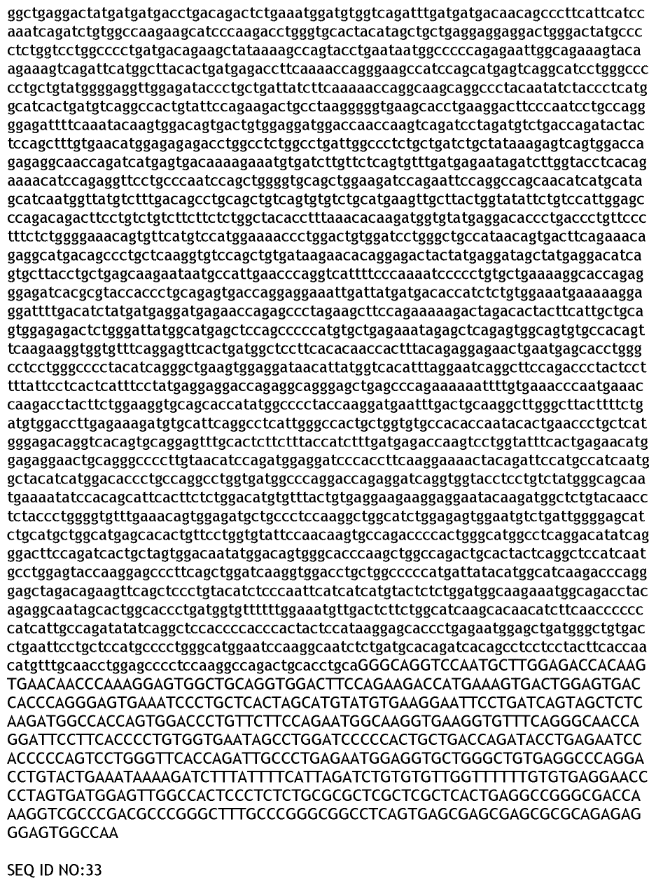

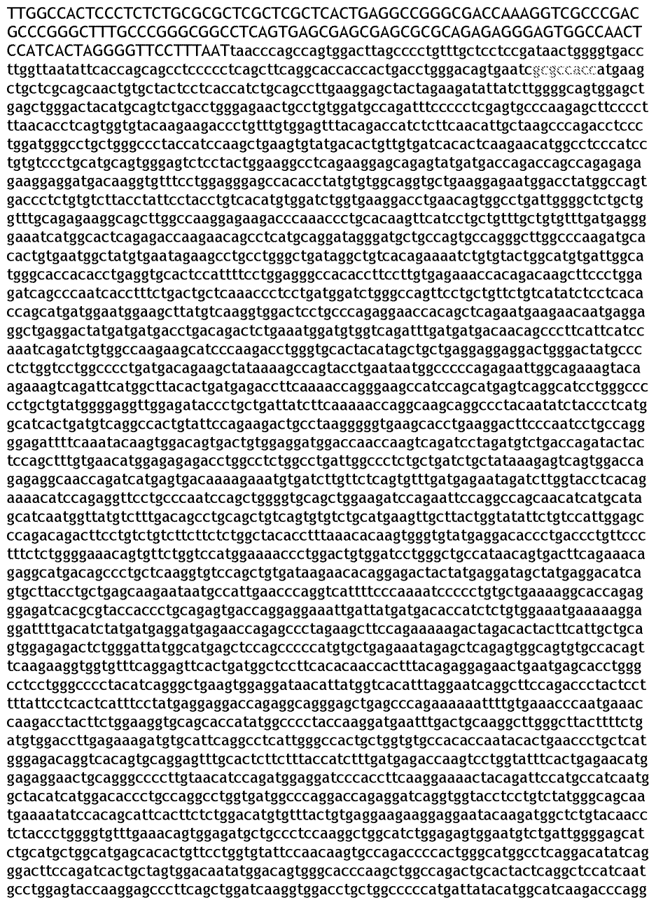

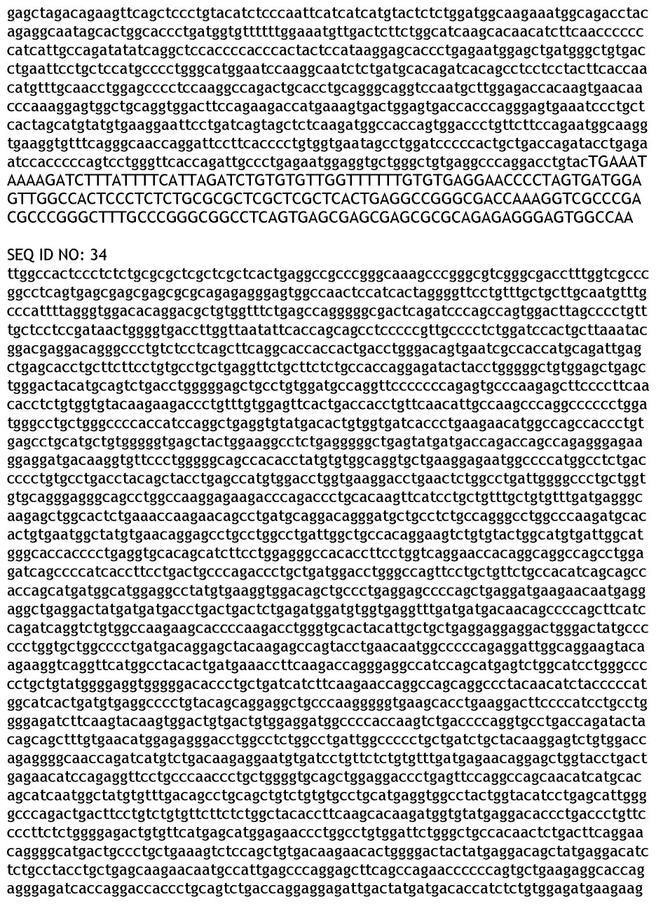

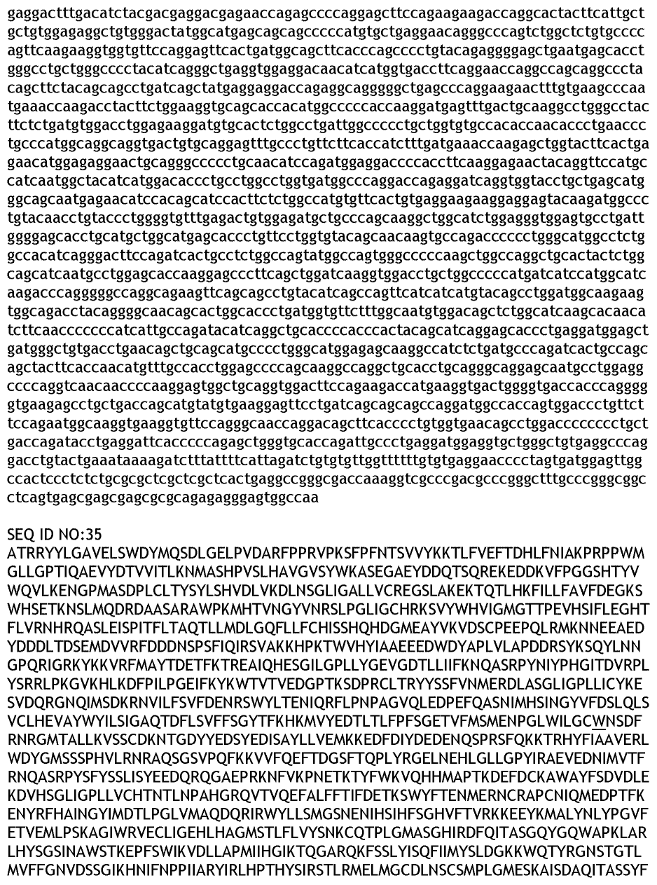

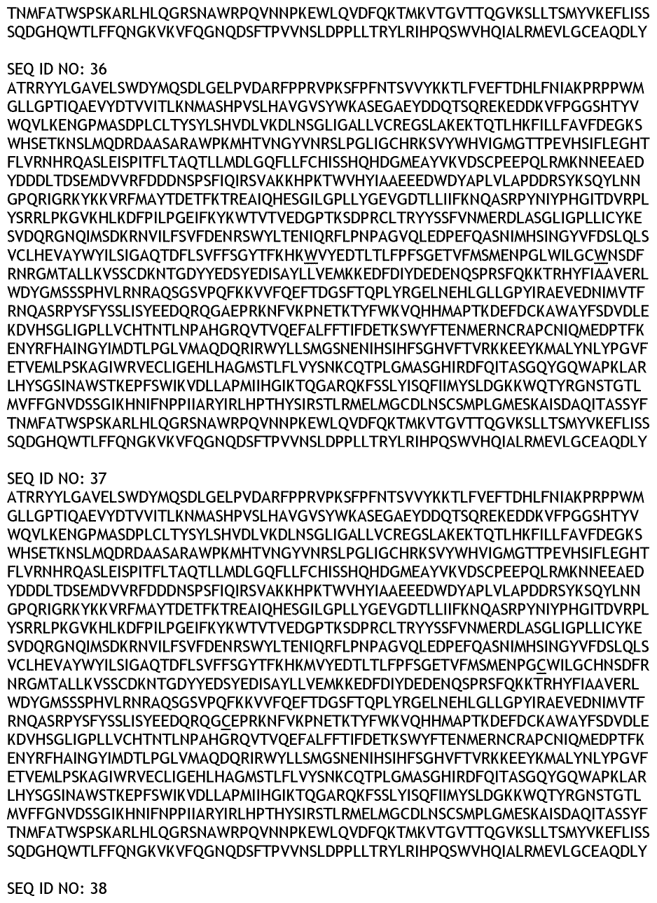

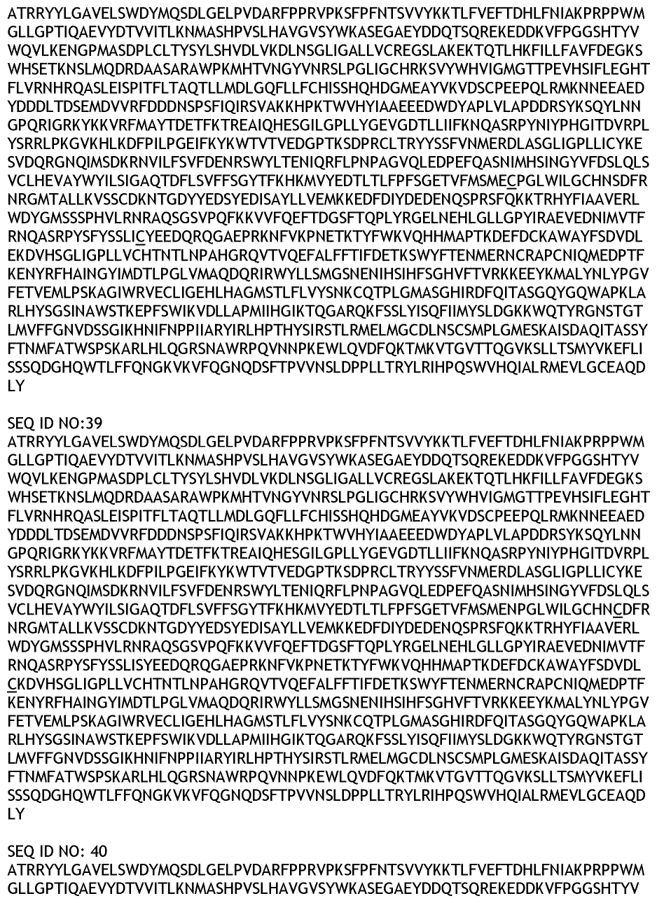

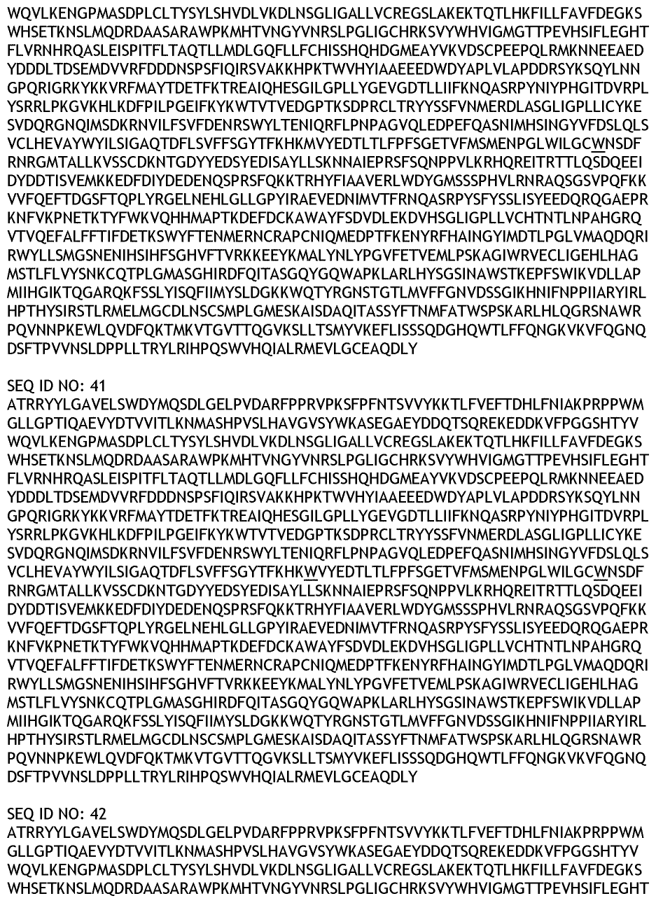

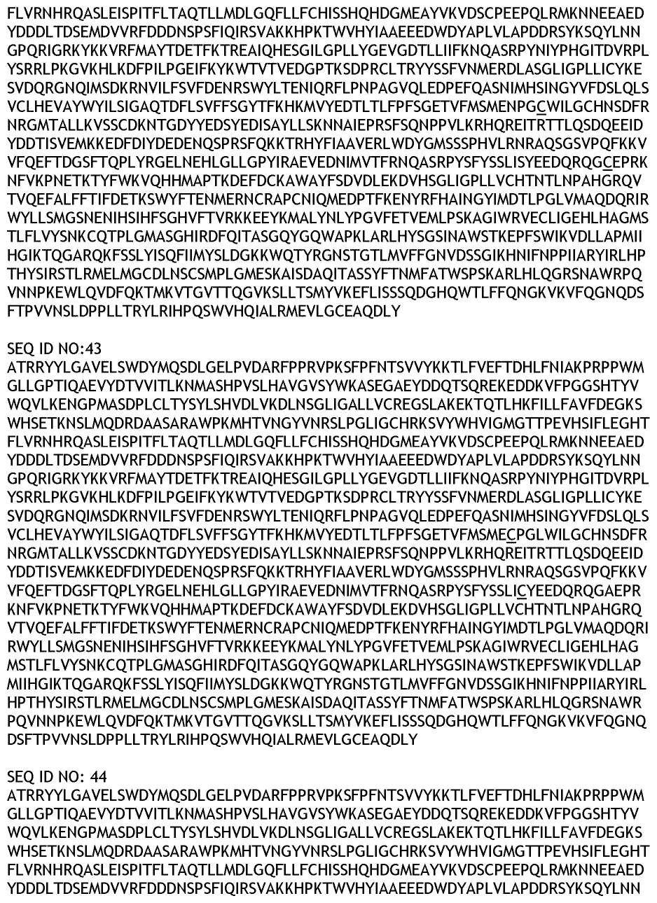

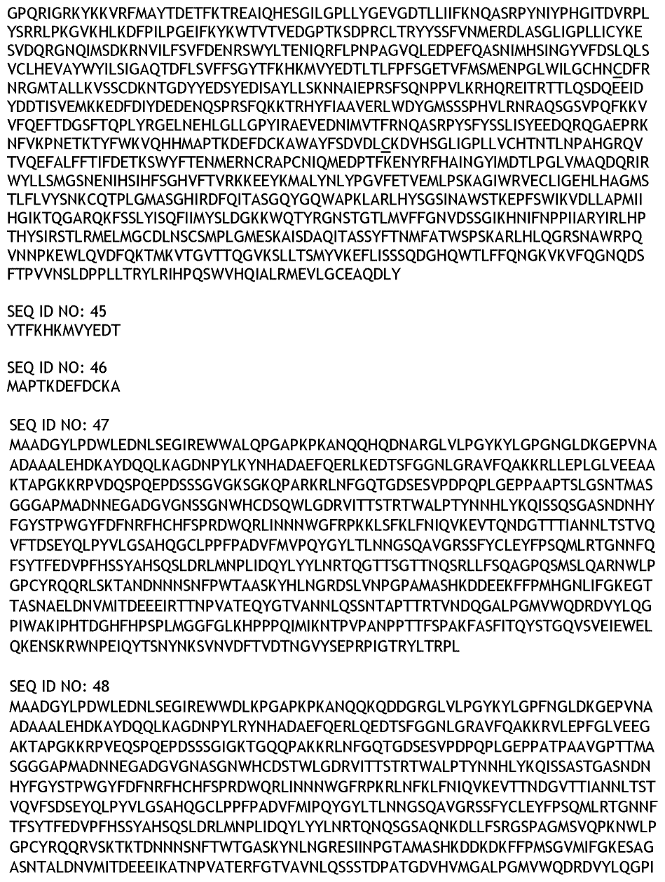

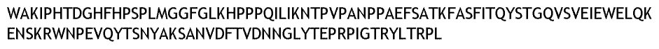

The plasma from injected and naïve animals were analysed for FVIII activity and human FVIII antigen level (assays as described in Example 1), and the ratio of FVIII activity to antigen level (i.e. specific activity) was calculated (FIG. 5).

### Numbered Aspects of the Invention

1. A Factor VIII polypeptide comprising a Factor VIII amino acid sequence, wherein the Factor VIII amino acid sequence comprises one or more substitution mutations at an inter-domain interface selected from the group consisting of:
a. the A1/A3 domain interface;
b. the A2/A3 domain interface; or
c. the A1/C2 domain interface,
   wherein:
(i) the one or more substitution mutations comprises substitution of an amino acid with a more hydrophobic amino acid; or
(ii) the one or more substitution mutations comprises substitution of a pair of amino acids in the respective domains with cysteine residues;
   and wherein the Factor VIII polypeptide has higher specific activity than a reference wild-type Factor VIII polypeptide.

2. A Factor VIII polypeptide comprising a Factor VIII amino acid sequence, wherein the Factor VIII amino acid sequence comprises one or more substitution mutations at an inter-domain interface selected from the group consisting of:
a. the A1/A3 domain interface;
b. the A2/A3 domain interface; or
c. the A1/C2 domain interface,
   wherein:
(i) the one or more substitution mutations comprises substitution of an amino acid with a more hydrophobic amino acid; or
(ii) the one or more substitution mutations comprises substitution of a pair of amino acids in respective domains with cysteine residues;
   and wherein the Factor VIII polypeptide has higher stability than a reference wild-type Factor VIII polypeptide.

3. A Factor VIII polypeptide comprising a Factor VIII amino acid sequence, wherein the Factor VIII amino acid sequence comprises one or more substitution mutations at an inter-domain interface selected from the group consisting of:
a. the A1/A3 domain interface;
b. the A2/A3 domain interface; or
c. the A1/C2 domain interface,
   wherein:
(i) the one or more substitution mutations comprises substitution of an amino acid with a more hydrophobic amino acid; or
(ii) the one or more substitution mutations comprises substitution of a pair of amino acids in respective domains with cysteine residues;
   and wherein the Factor VIII polypeptide is expressed at a higher level in a host cell than a reference wild-type Factor VIII polypeptide.

4. A Factor VIII polypeptide comprising a Factor VIII amino acid sequence, wherein the Factor VIII amino acid sequence comprises one or more substitution mutations selected from the group consisting of:
a. a substitution of an amino acid corresponding to M662 or H693 of SEQ ID NO: 1; or
b. a substitution of a pair of amino acids comprising a first amino acid and a second amino acid with cysteine residues, wherein:
   1. the first amino acid corresponds to M147, S149 or S289 of SEQ ID NO: 1 and the second amino acid corresponds to E1969, E1970 or N1977 of SEQ ID NO: 1;
   2. the first amino acid corresponds to T667, T669, N684, L687, I689, S695 or F697 of SEQ ID NO: 1 and the second amino acid corresponds to S1791, G1799, A1800, R1803, E1844, S1949, G1981, V1982, or Y1979 of SEQ ID NO: 1; or
   3. the first amino acid corresponds to A108, T118 or V137 of SEQ ID NO: 1 and the second amino acid corresponds to N2172, Q2329 or Y2332 of SEQ ID NO: 1.

5. The Factor VIII polypeptide of any one of aspects 2 to 4, wherein the Factor VIII polypeptide has higher specific activity relative to a reference wild-type Factor VIII polypeptide.

6. The Factor VIII polypeptide of aspect 1 or 5, wherein the Factor VIII polypeptide has a specific activity which is at least 1.1 fold, at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of the reference Factor VIII polypeptide.

7. The Factor VIII polypeptide of any one of aspects 1 or 3 to 5, wherein the Factor VIII polypeptide has higher stability relative to a reference wild-type Factor VIII polypeptide.

8. The Factor VIII polypeptide of aspect 2 or 7, wherein the Factor VIII polypeptide has a longer half-life relative to the reference wild-type Factor VIII polypeptide, optionally wherein the Factor VIII polypeptide has a longer half-life relative to the reference wild-type Factor VIII polypeptide when activated.

9. The Factor VIII polypeptide of aspect 8, wherein the Factor VIII polypeptide has a longer half-life which is at least 1.1, at least 1.2, at least 1.5, at least 1.7, at least 1.8, at least 2, at least 2.2, at least 2.5, at least 2.8 or at least 3 times the half-life of a reference wild-type Factor VIII polypeptide, and/or wherein the Factor VIII polypeptide has a half-life when activated which is at least 1.1, at least 1.2, at least 1.5, at least 1.7, at least 1.8, at least 2, at least 2.2, at least 2.5, at least 2.8 or at least 3 times the half-life of a reference wild-type Factor VIII polypeptide when activated.

10. The Factor VIII polypeptide of aspect 8 or 9, wherein the longer half-life is longer half-life in plasma.

11. The Factor VIII polypeptide of aspect 1, 2 or 4 to 6, wherein the Factor VIII polypeptide is expressed at a higher level in a host cell than a reference wild-type Factor VIII polypeptide.

12. The Factor VIII polypeptide of aspect 11, wherein the level of Factor VIII polypeptide that is expressed is the level of FVIII polypeptide secreted by a host cell.

13. The Factor VIII polypeptide of aspect 11 or 12, wherein the host cell is a human liver cell.

14. The Factor VIII polypeptide of aspect 13, wherein the human liver cell is an Huh7 cell.

15. The Factor VIII polypeptide of aspect 11, wherein the level of Factor VIII polypeptide that is expressed is in vivo expression.

16. The Factor VIII polypeptide of any one of aspects 1 to 15, wherein the Factor VIII polypeptide has higher specific activity and/or higher stability and/or is expressed at a higher level in a host cell than a reference Factor VIII polypeptide which comprises the Factor VIII amino acid sequence of the Factor VIII polypeptide but which does not comprise the one or more substitution mutations.

17. The Factor VIII polypeptide of aspect 16, wherein the reference Factor VIII polypeptide is the Factor VIII polypeptide of SEQ ID NO: 1, 3 or 5.

18. The Factor VIII polypeptide of any one of aspects 1 to 17, wherein the one or more substitution mutations at the inter-domain interface stabilise the interaction of the respective domains of the Factor VIII polypeptide when activated.

19. The Factor VIII polypeptide of aspect 18, wherein at least one of the one or more acid substitution mutations increases pi-stacking interactions between amino acid side chains of the respective domains.

20. The Factor VIII polypeptide of aspect 18 or 19, wherein at least one of the one or more substitution mutations increases hydrophobic packing between the amino acid side-chains of the respective domains.

21. The Factor VIII polypeptide of any one of aspects 18 to 20, wherein at least one of the one or more substitution mutations reduces steric clashing and/or unfavourable electrostatic interactions between amino acid side chains of the respective domains.

22. The Factor VIII polypeptide of any one of aspects 1 to 3 or 5 to 21, wherein the one or more substitution mutations comprise a substitution of one or more surface-inaccessible amino acids at an inter-domain interface.

23. The Factor VIII polypeptide of any one of aspects 1 to 3 or 5 to 22, wherein the amino acid substituted with a more hydrophobic amino acid is methionine or histidine.

24. The Factor VIII polypeptide of any one of aspects 1 to 3 or 5 to 23, wherein the amino acid substituted with a more hydrophobic amino acid is methionine corresponding to the amino acid at position 662 of SEQ ID NO: 1 or histidine corresponding to the amino acid at position 693 of SEQ ID NO: 1.

25. The Factor VIII polypeptide of any one of aspects 1 to 24, wherein the one or more substitution mutations does not comprise the M662C substitution.

26. The Factor VIII polypeptide of any one of aspects 1 to 25, wherein:
a. the one or more substitution mutations comprises substitution of methionine with tyrosine, isoleucine, leucine, phenylalanine or tryptophan; and/or
b. the one or more substitution mutations comprises substitution of histidine with glutamate, cysteine, valine, methionine, tyrosine, isoleucine, leucine, phenylalanine or tryptophan.

27. The Factor VIII polypeptide of any one of aspects 1 to 26, wherein the one or more substitution mutations comprises substitution of an amino acid with an aromatic amino acid.

28. The Factor VIII polypeptide of any one of aspects 1 to 27, wherein the one or more substitution mutations comprises the M662W substitution.

29. The Factor VIII polypeptide of any one of aspects 1 to 28, wherein the one or more substitution mutations comprises the H693W substitution.

30. The Factor VIII polypeptide of any one of aspects 1 to 29, wherein the one or more substitution mutations comprises the M662W and H693W substitutions.

31. The Factor VIII polypeptide of any one of aspects 1 to 30, wherein the one or more substitution mutations comprises substitution of a pair of amino acids in the respective domains with cysteine residues, wherein the cysteine residues form a disulphide bond between the respective domains.

32. The Factor VIII polypeptide of any one of aspects 1 to 31, wherein the one or more substitution mutations comprises substitution of a pair of amino acids in the respective domains with cysteine residues, wherein the pair of amino acids are in the A1 and A3 domains.

33. The Factor VIII polypeptide of any one of aspects 1 to 32, wherein the one or more substitution mutations comprises substitution of a pair of amino acids in the respective domains with cysteine residues, wherein the pair of amino acids comprises a first amino acid and a second amino acid, wherein the first amino acid corresponds to M147, S149 or S289 of SEQ ID NO: 1 and the second amino acid corresponds to E1969, E1970 or N1977 of SEQ ID NO: 1.

34. The Factor VIII polypeptide of any one of aspects 1 to 33, wherein the one or more substitution mutations comprises substitution of a pair of amino acids in the respective domains with cysteine residues, wherein the one or more substitution mutations comprises a pair of substitution mutations selected from the list consisting of (i) S289C and N1977C, (ii) M147C and E1970C, and (iii) S149C and E1969C.

35. The Factor VIII polypeptide of any one of aspects 1 to 31, wherein the one or more substitution mutations comprises substitution of a pair of amino acids in the respective domains with cysteine residues, wherein the pair of amino acids are in the A2 and A3 domains.

36. The Factor VIII polypeptide of any one of aspects 1 to 31 or 35, wherein the one or more substitution mutations comprises substitution of a pair of amino acids in the respective domains with cysteine residues, wherein the pair of amino acids comprises a first amino acid and a second amino acid, wherein the first amino acid corresponds to T667, T669, N684, L687, I689, 5695 or F697 of SEQ ID NO: 1 and the second amino acid corresponds to S1791, G1799, A1800, R1803, E1844, S1949, G1981, V1982, or Y1979 of SEQ ID NO: 1.

37. The Factor VIII polypeptide of any one of aspects 1 to 31, 35 or 36, wherein the one or more substitution mutations comprises substitution of a pair of amino acids in the respective domains with cysteine residues, wherein the one or more substitution mutations comprises a pair of substitution mutations selected from the list consisting of (i) T669C and V1982C, (ii) L687C and A1800C, (iii) I689C and G1799C, (iv) F697C and S1949C, (v) T667C and G1981C, (vi) T669C and Y1979C, (vii) N684C and S1791C, (viii) L687C and R1803C, and (ix) S695C and E1844C.

38. The Factor VIII polypeptide of any one of aspects 1 to 31, wherein the one or more substitution mutations comprises substitution of a pair of amino acids in the respective domains with cysteine residues, wherein the pair of amino acids are in the A1 and C2 domains.

39. The Factor VIII polypeptide of any one of aspects 1 to 31 or 38, wherein the one or more substitution mutations comprises substitution of a pair of amino acids in the respective domains with cysteine residues, wherein the pair of amino acids comprises a first amino acid and a second amino acid, wherein the first amino acid corresponds to A108, T118 or V137 of SEQ ID NO: 1 and the second amino acid corresponds to N2172, Q2329 or Y2332 of SEQ ID NO: 1.

40. The Factor VIII polypeptide of any one of aspects 1 to 31, 38 or 39, wherein the one or more substitution mutations comprises substitution of a pair of amino acids in the respective domains with cysteine residues, wherein the one or more substitution mutations comprises a pair of substitution mutations selected from the list consisting of (i) A108C and Q2329C, (ii) T118C and N2172C, and (iii) V137C and Y2332C.

41. The Factor VIII polypeptide of any one of aspects 1 to 40, wherein the one or more substitution mutations do not inhibit activation of the Factor VIII polypeptide.

42. The Factor VIII polypeptide of any one of aspects 1 to 41, wherein the Factor VIII amino acid sequence comprises an amino acid sequence that is at least 90%, at least 95%, at least 96%, at least 97%, at least 98% or at least 99% identical to the amino acid sequence set forth in SEQ ID NO: 1, 3 or 5, or an amino acid sequence that is at least 90%, at least 95%, at least 96%, at least 97%, at least 98% or at least 99% identical to an amino acid sequence comprising at least 1349 amino acids of SEQ ID NO: 1, 3 or 5.

43. The Factor VIII polypeptide of any one of aspects 1 to 42, wherein the Factor VIII polypeptide is a beta domain deleted (BDD) Factor VIII polypeptide.

44. The Factor VIII polypeptide of any one of aspects 1 to 43, wherein the Factor VIII amino acid sequence comprises amino acids corresponding to positions 1 to 722 and 1670 to 2332 of SEQ ID NO: 1.

45. The Factor VIII polypeptide of aspect 44, wherein the Factor VIII amino acid sequence comprises amino acids corresponding to positions 1 to 731 and 1670 to 2332 of SEQ ID NO: 1.

46. The Factor VIII polypeptide of any one of aspects 1 to 43, wherein the Factor VIII amino acid sequence is at least 90%, at least 95%, at least 96%, at least 97%, at least 98% or at least 99% identical to the amino acid sequence set forth in SEQ ID NO: 1.

47. The Factor VIII polypeptide of any one of aspects 1 to 46, wherein the Factor VIII amino acid sequence comprises the amino acid sequence set forth in SEQ ID NO: 28, or an amino acid sequence that is at least 90%, at least 95%, at least 96%, at least 97%, at least 98%, at least 99% or 100% identical to the amino acid sequence set forth in SEQ ID NO: 28.

48. A polynucleotide comprising a Factor VIII nucleotide sequence, wherein the Factor VIII nucleotide sequence encodes the Factor VIII polypeptide according to any one aspects 1 to 47.

49. The polynucleotide of aspect 48, wherein the Factor VIII nucleotide sequence is codon-optimised.

50. The polynucleotide of aspect 48 or 49, wherein the Factor VIII nucleotide sequence is codon-optimised for expression in human liver cells.

51. The polynucleotide of aspect 50, wherein the Factor VIII polypeptide encoded by the Factor VIII nucleotide sequence is expressed in human liver cells at higher levels compared to a reference wild-type Factor VIII nucleotide sequence.

52. The polynucleotide of aspect 50 or 51, wherein the Factor VIII polypeptide encoded by the Factor VIII nucleotide sequence is expressed in human liver cells at least 1.1 fold, at least 1.2 fold, at least 1.5 fold, at least 1.8 fold, at least 2 fold, at least 5 fold, at least 10 fold, at least 20 fold, at least 30 fold, at least 40 fold, or at least 50 fold higher compared to a reference wild-type Factor VIII nucleotide sequence.

53. The polynucleotide of aspect 51 or 52, wherein the reference wild-type Factor VIII nucleotide sequence is the Factor VIII nucleotide sequence of SEQ ID NO: 2.

54. The polynucleotide of any one of aspects 49 to 53, wherein the Factor VIII nucleotide sequence encoding the Factor VIII amino acid sequence comprises a nucleotide sequence that is at least 85%, at least 90%, at least 95%, at least 96%, at least 97%, at least 98%, at least 99% or 100% identical to the nucleotide sequence set forth in SEQ ID NO: 31, or a nucleotide sequence that is at least 85%, at least 90%, at least 95%, at least 96%, at least 97%, at least 98%, at least 99% or 100% identical to a nucleotide sequence comprising at least 4047 nucleotides of SEQ ID NO: 31.

55. The polynucleotide of any one of aspects 49 to 54, wherein the Factor VIII nucleotide sequence encoding the Factor VIII amino acid sequence comprises the nucleotide sequence set forth in SEQ ID NO:29 or a nucleotide sequence that is at least 85%, at least 90%, at least 95%, at least 96%, at least 97%, at least 98%, at least 99% or 100% identical to the nucleotide sequence set forth in SEQ ID NO:29.

56. A recombinant AAV construct which comprises a polynucleotide comprising a Factor VIII nucleotide sequence according to any one of aspects 49 to 55.

57. The recombinant AAV construct of aspect 56, which is less than 4900 nucleotides in length.

58. The recombinant AAV construct of aspect 57, which is between 4700 and 4900, between 4700 and 4850, between 4700 and 4800, between 4700 and 4750 or around 4713 nucleotides in length.

59. The recombinant AAV construct of any one of aspects 56-58, wherein the recombinant AAV construct is single-stranded.

60. The recombinant AAV construct of any one of aspects 56 to 59, wherein the polynucleotide comprising a Factor VIII nucleotide sequence is operably linked to a transcription regulatory element and/or a polyA sequence.

61. The recombinant AAV construct of aspect 60, wherein the transcriptional regulatory element has the sequence set forth in SEQ ID NOs: 13 or 14.

62. The recombinant AAV construct of aspect 60 or 61, wherein the polyA sequence has a sequence set forth in SEQ ID NO: 22.

63. The recombinant AAV construct of any one of aspects 56 to 62, further comprising a nucleotide sequence encoding a signal peptide.

64. The recombinant AAV construct of aspect 63, wherein the signal peptide is a wild-type Factor VIII signal peptide.

65. The recombinant AAV construct of aspect 64, wherein the signal peptide comprises SEQ ID NO: 15 or wherein the nucleotide sequence encoding a signal peptide comprises SEQ ID NO: 16 or SEQ ID NO: 17.

66. The recombinant AAV construct of aspect 63, wherein the signal peptide is not a wild-type Factor VIII signal peptide.

67. The recombinant AAV construct of aspect 66, wherein the signal peptide comprises SEQ ID NO: 18 or 20, or wherein the nucleotide sequence encoding the signal peptide comprises SEQ ID NO: 19 or 21.

68. The recombinant AAV construct of any one of aspects 56 to 67, further comprising one or two ITR(s).

69. The recombinant AAV construct of aspect 68, wherein the nucleotide sequence of the or each ITR is fewer than 157, fewer than 154, or around 145 nucleotides in length.

70. The recombinant AAV construct of aspect 68 or 69, wherein the or each ITR is a wild-type ITR and/or wherein the or each ITR is an AAV2 ITR.

71. The recombinant AAV construct of any one of aspects 68 to 70, wherein the nucleotide sequence of the or each ITR comprises a nucleotide sequence of SEQ ID NO: 23 or 24.

72. The recombinant AAV construct of any one of aspects 56 to 71, wherein the AAV construct is an AAV genome.

73. The recombinant AAV construct of any one of aspects 56 to 72, wherein the AAV construct comprises or consists of the sequence set forth in SEQ ID NO: 27.

74. An AAV viral particle comprising the recombinant AAV construct according to any one of aspects 56 to 73.

75. The AAV viral particle of aspect 74, wherein the viral particle comprises a capsid.

76. The AAV viral particle of aspect 75, wherein the capsid is selected from the group consisting of:
(i) a capsid comprising a sequence which is at least 96%, at least 98%, at least 99%, at least 99.5%, at least 99.8% or 100% identical to SEQ ID NO: 25;
(ii) a capsid comprising a sequence which is at least 96%, at least 98%, at least 99%, at least 99.5%, at least 99.8%, or 100% identical to SEQ ID NO: 26;
(iii) a liver-tropic capsid; and
(iv) an AAV5 capsid.

77. The AAV viral capsid according to aspect 75, wherein the capsid is selected from the group consisting of:
(i) a capsid comprising a sequence which is at least 96%, at least 98%, at least 99%, at least 99.5%, at least 99.8% or 100% identical to SEQ ID NO: 47; and
(ii) a capsid comprising a sequence which is at least 96%, at least 98%, at least 99%, at least 99.5%, at least 99.8% or 100% identical to SEQ ID NO: 48; and
(iii) an AAV6 capsid.

77. A composition comprising the Factor VIII polypeptide, polynucleotide, recombinant AAV construct, or AAV viral particle according to any one of aspects 1 to 76, and a pharmaceutically acceptable excipient.

78. The Factor VIII polypeptide, polynucleotide, recombinant AAV construct, AAV viral particle or composition according to any one of aspects 1 to 77 for use in a method of treatment.

79. The Factor VIII polypeptide, polynucleotide, recombinant AAV construct, AAV viral particle or composition for use according to aspect 78, wherein the method of treatment comprises administering an effective amount of the Factor VIII polypeptide, polynucleotide, recombinant AAV construct or AAV viral particle of any one of aspects 1 to 77 to a patient.

80. A method of treatment comprising administering an effective amount of the Factor VIII polypeptide, polynucleotide, recombinant AAV construct or AAV viral particle or composition according to any one of aspects 1 to 77 to a patient.

81. Use of a Factor VIII polypeptide, polynucleotide, recombinant AAV construct, AAV viral particle or composition according to any one of aspects 1 to 77 in the manufacture of a medicament for use in a method of treatment.

82. The use of aspect 80, wherein the method of treatment comprises administering an effective amount of the Factor VIII polypeptide, polynucleotide, recombinant AAV construct AAV viral particle or composition according to any one of aspects 1 to 77 to a patient.

83. The Factor VIII polypeptide, polynucleotide, recombinant AAV construct AAV viral particle or composition for use, method or use according to any one of aspects 78 to 82, wherein the method of treatment is a method of treating haemophilia.

84. The Factor VIII polypeptide, polynucleotide, recombinant AAV construct, AAV viral particle or composition for use, method or use according to any one of aspects 78 to 83, wherein the method of treatment is a method of treating haemophilia A.

1. A Factor VIII polypeptide comprising a Factor VIII amino acid sequence, wherein the Factor VIII amino acid sequence comprises one or more substitution mutations, wherein the one or more substitution mutations comprises a substitution of an amino acid corresponding to M662 of SEQ ID NO:1, wherein the one or more substitution mutations does not comprise the M662C substitution.

2. The Factor VIII polypeptide of claim 1, wherein:
(i) the Factor VIII polypeptide has higher specific activity relative to a reference wild-type Factor VIII polypeptide; and/or
(ii) the Factor VIII polypeptide has higher stability relative to a reference wild-type Factor FVIII polypeptide; and/or
(iii) the Factor VIII polypeptide has a longer half-life relative to a reference wild-type Factor VIII polypeptide, optionally wherein the Factor VIII polypeptide has a longer half-life relative to the reference wild-type Factor VIII polypeptide when activated; and/or
(iv) the Factor VIII polypeptide is expressed at a higher level in a host cell than a reference wild-type Factor VIII polypeptide.

3. The Factor VIII polypeptide of claim 2, wherein the Factor VIII polypeptide has a specific activity which is at least 1.1 fold, at least 1.2 fold, at least 1.5 fold, at least 1.7 fold, at least 1.8 fold, at least 2 fold, at least 2.2 fold, at least 2.5 fold, at least 3 fold, at least 3.5 fold, at least 4 fold, at least 4.5 fold, at least 5 fold, or at least 5.5 fold higher than the specific activity of the reference Factor VIII polypeptide.

4. The Factor VIII polypeptide of claim 2 or 3, wherein the Factor VIII polypeptide has a longer half-life which is at least 1.1, at least 1.2, at least 1.5, at least 1.7, at least 1.8, at least 2, at least 2.2, at least 2.5, at least 2.8 or at least 3 times the half-life of a reference wild-type Factor VIII polypeptide, and/or wherein the Factor VIII polypeptide has a half-life when activated which is at least 1.1, at least 1.2, at least 1.5, at least 1.7, at least 1.8, at least 2, at least 2.2, at least 2.5, at least 2.8 or at least 3 times the half-life of a reference wild-type Factor VIII polypeptide when activated, optionally, wherein the longer half-life is longer half-life in plasma.

5. The Factor VIII polypeptide of any one of claims 2 to 4, wherein:
(i) the level of Factor VIII polypeptide that is expressed is the level of FVIII polypeptide secreted by a host cell, optionally wherein the host cell is a human liver cell, optionally wherein the human liver cell is an Huh7 cell; and/or
(ii) the level of Factor VIII polypeptide that is expressed is in vivo expression.

6. The Factor VIII polypeptide of any one of claims 2 to 5, wherein the Factor VIII polypeptide has higher specific activity and/or higher stability and/or is expressed at a higher level in a host cell than a reference Factor VIII polypeptide which comprises the Factor VIII amino acid sequence of the Factor VIII polypeptide but which does not comprise the one or more substitution mutations, optionally wherein the reference Factor VIII polypeptide is the Factor VIII polypeptide of SEQ ID NO: 1, 3 or 5.

7. The Factor VIII polypeptide of any one of claims 1 to 6, wherein the one or more substitution mutations comprises substitution of methionine with tryptophan, tyrosine, isoleucine, leucine or phenylalanine.

8. The Factor VIII polypeptide of any one of claims 1 to 7, wherein the one or more substitution mutations comprises a substitution of an amino acid corresponding to M662 of SEQ ID NO:1 with an aromatic amino acid.

9. The Factor VIII polypeptide of any one of claims 1 to 8, wherein the one or more substitution mutations comprises the M662W substitution.

10. The Factor VIII polypeptide of any one of claims 1 to 9, wherein the one or more substitution mutations comprises the M662W and H693W substitutions.

11. The Factor VIII polypeptide of any one of claims 1 to 10, wherein the one or more substitution mutations do not inhibit activation of the Factor VIII polypeptide.

12. The Factor VIII polypeptide of any one of claims 1 to 11, wherein the Factor VIII amino acid sequence comprises an amino acid sequence that is at least 90%, at least 95%, at least 96%, at least 97%, at least 98% or at least 99% identical to the amino acid sequence set forth in SEQ ID NO: 1, 3 or 5, or an amino acid sequence that is at least 90%, at least 95%, at least 96%, at least 97%, at least 98% or at least 99% identical to an amino acid sequence comprising at least 1349 amino acids of SEQ ID NO: 1, 3 or 5.

13. The Factor VIII polypeptide of any one of claims 1 to 12, wherein the Factor VIII polypeptide is a beta domain deleted (BDD) Factor VIII polypeptide.

14. The Factor VIII polypeptide of any one of claims 1 to 13, wherein the Factor VIII amino acid sequence comprises amino acids corresponding to positions 1 to 731 and 1670 to 2332 of SEQ ID NO:1.

15. The Factor VIII polypeptide of any one of claims 1 to 14, wherein the Factor VIII amino acid sequence comprises the amino acid sequence set forth in SEQ ID NO: 28, or an amino acid sequence that is at least 90%, at least 95%, at least 96%, at least 97%, at least 98%, at least 99% or 100% identical to the amino acid sequence set forth in SEQ ID NO: 28.

16. A Factor VIII polypeptide comprising a Factor VIII amino acid sequence, wherein the Factor VIII amino acid sequence comprises one or more substitution mutations at an inter-domain interface selected from the group consisting of:
a. the A1/A3 domain interface;
b. the A2/A3 domain interface; or
c. the A1/C2 domain interface,
wherein:
(iii) the one or more substitution mutations comprises substitution of an amino acid with a more hydrophobic amino acid; or
(iv) the one or more substitution mutations comprises substitution of a pair of amino acids in the respective domains with cysteine residues;
and wherein:
(A) the Factor VIII polypeptide has higher specific activity than a reference wild-type Factor VIII polypeptide; and/or
(B) the Factor VIII polypeptide has higher stability than a reference wild-type Factor VIII polypeptide; and/or
(C) the Factor VIII polypeptide is expressed at a higher level in a host cell than a reference wild-type Factor VIII polypeptide.

17. A Factor VIII polypeptide comprising a Factor VIII amino acid sequence, wherein the Factor VIII amino acid sequence comprises one or more substitution mutations selected from the group consisting of:
a. a substitution of an amino acid corresponding to M662 or H693 of SEQ ID NO: 1; or
b. a substitution of a pair of amino acids comprising a first amino acid and a second amino acid with cysteine residues, wherein:
i. the first amino acid corresponds to M147, S149 or S289 of SEQ ID NO: 1 and the second amino acid corresponds to E1969, E1970 or N1977 of SEQ ID NO: 1;
ii. the first amino acid corresponds to T667, T669, N684, L687, I689, S695 or F697 of SEQ ID NO: 1 and the second amino acid corresponds to S1791, G1799, A1800, R1803, E1844, S1949, G1981, V1982, or Y1979 of SEQ ID NO: 1; or
iii. the first amino acid corresponds to A108, T118 or V137 of SEQ ID NO: 1 and the second amino acid corresponds to N2172, Q2329 or Y2332 of SEQ ID NO: 1.

18. A polynucleotide comprising a Factor VIII nucleotide sequence, wherein the Factor VIII nucleotide sequence encodes the Factor VIII polypeptide according to any one of claims 1 to 17.

19. The polynucleotide of claim 18, wherein the Factor VIII nucleotide sequence encoding the Factor VIII amino acid sequence comprises a nucleotide sequence that is at least 85%, at least 90%, at least 95%, at least 96%, at least 97%, at least 98%, at least 99% or 100% identical to the nucleotide sequence set forth in SEQ ID NO: 31, or a nucleotide sequence that is at least 85%, at least 90%, at least 95%, at least 96%, at least 97%, at least 98%, at least 99% or 100% identical to a nucleotide sequence comprising at least 4047 nucleotides of SEQ ID NO: 31 or SEQ ID NO: 29.

20. A recombinant AAV construct comprising a polynucleotide comprising a Factor VIII nucleotide sequence according to claim 18 or 19.

21. An AAV viral particle comprising the recombinant AAV construct according to claim 20.

22. A composition comprising the Factor VIII polypeptide, polynucleotide, recombinant AAV construct or AAV viral particle according to any one of claims 1 to 21, and a pharmaceutically acceptable excipient.

23. The Factor FVIII polypeptide, polynucleotide, recombinant AAV construct, AAV viral particle or composition according to any one of claims 1 to 22 for use in a method of treatment.

24. The Factor FVIII polypeptide, polynucleotide, recombinant AAV construct, AAV viral particle or composition according to any one of claims 1 to 22 for use in a method of treating haemophilia A, wherein said method comprises administering an effective amount of the Factor VIII polypeptide, polynucleotide, recombinant AAV construct AAV viral particle or composition of any one of claims 1 to 22 to a patient.

25. A method of treating haemophilia A, wherein said method comprises administering an effective amount of the Factor VIII polypeptide, polynucleotide, recombinant AAV construct AAV, AAV viral particle or composition of any one of claims 1 to 22 to a patient.
